(19) United States
(12) Patent Application Publication
(10) Pub. No.: US 2024/0317749 A1
(43) Pub. Date: Sep. 26, 2024

(54) THERAPEUTIC COMPOUNDS

(71) Applicant: TAXIS PHARMACEUTICALS, INC., Monmouth Junction, NJ (US)

(72) Inventors: Ajit K. PARHI, Monmouth Junction, NJ (US); Yi YUAN, Monmouth Junction, NJ (US); Yongzheng ZHANG, Monmouth Junction, NJ (US); Jesus ROSADO, Monmouth Junction, NJ (US); Pratik DATTA, Monmouth Junction, NJ (US)

(73) Assignee: TAXIS PHARMACEUTICALS, INC., Monmouth Junction, NJ (US)

(21) Appl. No.: 18/572,411

(22) PCT Filed: Jun. 21, 2022
(86) PCT No.: PCT/US2022/034374

§ 371 Date: Dec. 20, 2023

Related U.S. Application Data
(60) Provisional application No. 63/213,519, filed on Jun. 22, 2021.

(51) Int. Cl. C07D 471/14; A61K 31/519; A61P 31/04; C07D 487/04

(52) U.S. Cl. CPC C07D 471/14; A61K 31/519; A61P 31/04; C07D 487/04

(57) ABSTRACT

Disclosed herein are compounds of formula I: and salts thereof. Also disclosed are compositions comprising compounds of formula I and methods using compounds of formula I.

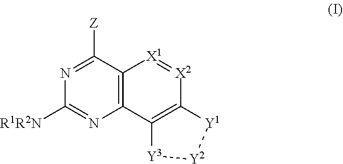

### CROSS-REFERENCE TO RELATED APPLICATIONS

This application claims priority to U.S. Provisional Application No. 63/213,519, filed Jun. 22, 20021. The entire content of the application referenced above is hereby incorporated by reference herein.

### BACKGROUND OF THE INVENTION

Bacterial resistance has been observed to every antibiotic introduced into the clinic, including drugs-of-last resort such as vancomycin, daptomycin, and colistin. The increased rate of resistance, coupled with declining trends in discovery and development of new antibiotics, foreshadows a crisis. Pathogens now exist that are resistant to all or almost all available antibiotics, resulting in an increased number of deaths from bacterial infections. According to the CDC, each year in the U.S., at least 2.8 million people are infected with antibiotic-resistant bacteria or fungi, and more than 35,000 people die as a result. Without improvements in antibacterial drug discovery and development this problem is only expected to get worse. It has been predicted that by 2050 10 million people worldwide will die per year from drug-resistant bacterial infections.

ESKAPE pathogens: Enterococcus faecium, Staphylococcus aureus, Klebsiella pneumoniae/Escherichia coli (Enterobacteriacaea), Acinetobacter baumannii, Pseudomonas aeruginosa, and Enterobacter species cause the most hospital-acquired infections and frequently “escape” the action of traditional therapeutics. The prevalence of multi-drug resistant (MDR) Gram-negative infections has continued to increase. The latest example comes from the observed rapid spread of multidrug-resistant gram-negative bacteria among patients in dedicated coronavirus disease care units in a hospital in Maryland, USA, during May-June 2020. The World Health Organization (WHO) issued a list of antibiotic-resistant ‘Priority Pathogens’ in 2017 for which new antibiotics are urgently needed. The most critical group of all includes multidrug resistant bacteria that pose a particular threat in hospitals, nursing homes, and among patients whose care requires devices such as ventilators and blood catheters. They include Acinetobacter, Pseudomonas and various Enterobacteriaceae (including Klebsiella, E. coli, Serratia, and Proteus). They can cause severe and often deadly infections such as bloodstream infections and pneumonia. The second and third most critical groups list high and medium priority bacteria that are becoming increasingly drug-resistant such as Neisseria gonorrhoeae. Sexually transmitted infections (STIs) caused by the bacteria N. gonorrhoeae (gonococcal infections) have increased 63% since 2014 and are a cause of sequelae including pelvic inflammatory disease, ectopic pregnancy, and infertility and can facilitate transmission of human immunodeficiency virus (HIV). N. gonorrhoeae's ability to acquire antimicrobial resistance influences treatment recommendations and complicates control. In 2010, CDC recommended the combination of ceftriaxone and azithromycin for treatment of uncomplicated gonococcal infections. However, continued low incidence of ceftriaxone resistance and the increased incidence of azithromycin resistance, has led to reevaluation of this recommendation. Azithromycin resistance in N. gonorrhoeae is an increasing concern. Genomic epidemiology data confirm that azithromycin resistance can result from multiple mechanisms. Nationally, the percentage of N. gonorrhoeae isolates with reduced susceptibility (MIC≥2.0 μg/mL) increased more than sevenfold over 5 years (from 0.6% in 2013 to 4.6% in 2018). During 2018, the proportion of Gonococcal Isolate Surveillance Project (GISP) isolates with an azithromycin alert value was 8.6% among men who have sex with men, compared with 2.9% among men who have sex with women only. Studies have associated development of reduced azithromycin susceptibility with azithromycin exposure among patients with N. gonorrhoeae infection. No new class of antibiotics effective against Gram-negative bacteria has been introduced into the clinic since the fluoroquinolones in 1968, and clinicians have had to rely on later generation broad-spectrum antibiotics (that is, antibiotics effective against both Gram-positive and Gram-negative pathogens), particularly the carbapenems. Unfortunately carbapenem-resistance is also on the rise, and infections caused by carbapenem-resistant Enterobacteriaceae (CRE) are estimated to have as much as 50% higher mortality rates than carbapenem-susceptible infections. With the increase in carbapenem-resistant infections, colistin, a drug that was previously removed from the clinic due to high toxicity, has been re-introduced to the clinic. However, colistin resistance is emerging and spread easily via horizontal gene transfer of plasmids. Pathogens that are resistant to both colistin and carbapenems are challenging to treat, with mortality rates ranging from 20-70%. With the increase in carbapenem-resistant and colistin-resistant infections, new broad-spectrum antibiotics will need to be developed to combat the rise of Gram-positive and Gram-negative antibiotic resistance.

Accordingly, there is a need for therapeutic agents and methods for treating and/or preventing bacterial infections.

### SUMMARY OF THE INVENTION

Compounds disclose herein, have been demonstrated to have antibacterial activity against a variety of bacteria.

Accordingly, one embodiment provides a compound of formula I:

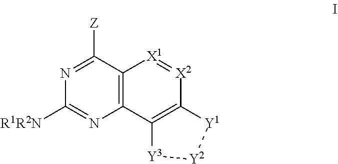

wherein;
R¹ is hydrogen, (C₁-C₆)alkyl, (C₂-C₆)alkenyl, (C₂-C₆)alkynyl, —C(═O)R^{a}, —C(═O)N(R^{a})₂, —S(═O)₂N(R^{a})₂, —C(═NR^{a})N(R^{a})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₂-C₆)alkynyl, (C₂-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, phenyl, and —OP(═O)(OH)₂, wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy; and
R² is hydrogen, (C₁-C₆)alkyl, (C₂-C₆)alkenyl, —C(═O)R^{b}, —C(═O)N(R^{b})₂, —S(═O)₂N(R^{b})₂, —C(═NR^{b})N(R^{b})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₂-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{b})₂, —NR^{b}C(═NR^{b})N(R^{b})₂, —C(═NR^{b})N(R^{b})₂, and —OP(═O)(OH)₂; or
R¹ and R² together with the nitrogen to which they are attached form a heterocyclyl, wherein the heterocycly is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{ab})₂, —NR^{ab}C(═NR^{ab})N(R^{ab})₂. —C(═NR^{ab})N(R^{ab})₂, and —OP(═O)(OH)₂,
Z is —OR^{c}, —N(R^{c})₂, —SR^{c}, —NR^{c}C(═NR^{c})N(R^{c})₂, —C(═NR^{c})N(R^{c})₂, hydrogen, (C₁-C₆)alkyl, (C₁-C₆)alkenyl, —C(═O)R^{c}, —C(═O)N(R^{c})₂, —S(═O)₂N(R^{c})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{c})₂, —NR^{c}C(═NR^{c})N(R^{c})₂, —C(═NR^{c})N(R^{c})₂, and —OP(═O)(OH)₂;
X¹ is CR³ or N;
X² is CR⁴ or N;
R³ is hydrogen, halo, —OH, —NO₂, —CN, (C₁-C₆)alkoxy, (C₁-C₆)alkyl, (C₁-C₆)alkenyl, (C₂-C₆)alkynyl —C(═O)R^{d}, —C(═O)N(R^{d})₂, —S(═O)₂N(R^{d})₂, —NR^{d}C(═NR^{d})N(R^{d})₂, —C(═NR^{d})N(R^{d})₂, or —N(R^{d})₂, wherein the (C₁-C₆)alkoxy, (C₁-C₆)alkyl, (C₂-C₆)alkenyl or (C₂-C₆)alkynyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{d})₂, —NR^{d}C(═NR^{d})N(R^{d})₂, —C(═NR^{d})N(R^{d})₂, and —OP(═O)(OH)₂;
R⁴ is hydrogen, halo, —OH, —NO₂, —CN, (C₁-C₆)alkoxy, (C₁-C₆)alkyl, (C₂-C₆)alkenyl, (C₂-C₆)alkynyl, —C(═O)R^{e}, —C(═O)N(R^{e})₂, —S(═O)₂N(R^{e})₂, —NR^{e}C(═NR^{e})N(R^{e})₂, —C(═NR^{e})N(R^{e})₂, or —N(R^{e})₂, wherein the (C₁-C₆)alkoxy, (C₁-C₆)alkyl, (C₂-C₆)alkenyl or (C₂-C₆)alkynyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{e})₂, —NR^{e}C(═NR^{e})N(R^{e})₂, —C(═NR^{e})N(R^{e})₂, and —OP(═O)(OH)₂;
Y¹ is NR^{5a}, Y² is N or CR^{6b}, Y³ is N or CR^{7b}, the dashed bond between Y¹ and Y² is a single bond, and the dashed bond between Y² and Y³ is a double bond; or
Y³ is NR^{7a}, Y¹ is N or CR^{5b}, Y² is N or CR^{6b}, the dashed bond between Y¹ and Y² is a double bond, and the dashed bond between Y² and Y³ is a single bond;
R^{5a} and R^{7a} are each independently (C₁-C₁₀)alkyl, (C₁-C₁₀)alkenyl, —W—(C₃-C₇)carbocyclyl, —W-aryl, W-heterocyclyl, -or W-heteroaryl, wherein the (C₁-C₁₀)alkyl, (C₁-C₁₀)alkenyl, —W—(C₃-C₇)carbocyclyl, —W-aryl, W-heterocyclyl, —W-heteroaryl, is optionally substituted with one or more (e.g., 1, 2, 3, 4, or 5) groups independently selected from Q;
R^{5b}, R^{6b}, and R^{7b} are each independently hydrogen, halo, —OH, —NO₂, —CN, (C₁-C₆)alkoxy, (C₁-C₆)alkyl, (C₁-C₆)alkenyl, —C(═O)R^{f}, —C(═O)N(R^{f})₂, —S(═O)₂N(R^{f})₂, —NR^{f}C(═NR^{f})N(R^{f})₂, —C(═NR^{f})N(R^{f})₂, or —N(R^{f})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo. —OH, —NO₂, —CN, (C₁-C₄)alkoxy, (C₁-C₄)haloalkoxy, —N(R^{f})₂, —NR^{f}C(═NR^{f})N(R^{f})₂, —C(═NR^{f})N(R^{f})₂, and —OP(═O)(OH)₂;
W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—;
each Q is independently halo, —OH, —NO₂, —CN, (C₁-C₆)alkyl, (C₂-C₆)alkenyl, (C₂-C₆)alkynyl, (C₁-C₆)haloalkyl, (C₁-C₆)alkoxy, (C₁-C₆)haloalkoxy, C(═O)R^{q}, —C(═O)N(R^{q})₂, —N(R^{q})₂, —S(═O)₂N(R^{q})₂, aryl, —O-aryl, heteroaryl, or —O-heteroaryl, wherein the (C₁-C₆)alkyl, (C₂-C₆)alkenyl, (C₂-C₆)alkynyl, (C₁-C₆)haloalkyl, (C₁-C₆)alkoxy, (C₁-C₆)haloalkoxy, aryl, -Oaryl, heteroaryl, or -Oheteroaryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q}, —C(═O)O(R^{q}), —C(═O)N(R^{q})₂, —S(═O)₂N(R^{q})₂, aryl and heteroaryl, wherein the aryl or heteroaryl is optionally substituted with one or more (e.g., 1, 2, 3, 4, or 5) Q¹;
each Q¹ is independently halo, —OH, —NO₂, —CN, (C₁-C₄)alkyl, (C₁-C₄)haloalkyl, (C₁-C₄)alkoxy, or (C₁-C₄)haloalkoxy;
each R^{a}, R^{b}, R^{ab}, R^{c}, R^{d}, R^{e}, and R^{f}, is independently hydrogen, (C₁-C₆)alkyl, (C₁-C₆)alkenyl, (C₁-C₆)haloalkyl, (C₃-C₇)carbocyclyl, or aryl;
each R^{w} is independently hydrogen or (C₁-C₆)alkyl;
R^{q} is hydrogen, (C₁-C₆)alkyl, (C₂-C₆)alkenyl, (C₁-C₆)haloalkyl (C₃-C₇)carbocyclyl or aryl, wherein the aryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q1}, —C(═O)O(R^{q1}), —C(═O)N(R^{q1})₂, and —S(═O)₂N(R^{q1})₂; and
each R^{q1} is independently hydrogen, (C₁-C₆)alkyl, (C₁-C₆)alkenyl, (C₁-C₆)haloalkyl, (C₃-C₇)carbocyclyl, or aryl;
or a salt thereof.

One embodiment provides a compound of formula I:

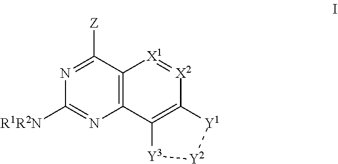

wherein;
R¹ is hydrogen, (C₁-C₆)alkyl, (C₁-C₆)alkenyl, —C(═O)R^{a}, —C(═O)N(R^{a})₂, —S(═O)₂N(R^{a})₂, —C(═NR^{a})N(R^{a})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₁-C₆)alkenyl, (C₃-C₇)carbocyclyl, aryl, heterocyclyl, heteroaryl·(C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, and —OP(═O)(OH)₂; and
R² is hydrogen, (C₁-C₆)alkyl, (C₁-C₆)alkenyl, —C(═O)R^{b}, —C(═O)N(R^{b})₂, —S(═O)₂N(R^{b})₂, —C(═NR^{b})N(R^{b})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{b})₂, —NR^{b}C(═NR^{b})N(R^{b})₂, —C(═NR^{b})N(R^{b})₂, and —OP(═O)(OH)₂; or
R¹ and R² together with the nitrogen to which they are attached form a heterocyclyl, wherein the heterocycly is optionally substituted with one or more (e.g., 1, 2, 3, 4, or 5) groups independently selected from the group consisting of halo. —OH, —NO₂, —CN, (C₁-C₄)alkyl, (C₁-C₄)haloalkyl, (C₁-C₄)alkoxy, (C₁-C₄)haloalkoxy, —N(R^{ab})₂, —NR^{ab}C(═NR^{ab})N(R^{ab}), —C(═NR^{ab})N(R^{ab})₂, and —OP(═O)(OH)₂,
Z is —OR^{c}, —N(R)₂, —SR^{c}, —NR^{c}C(═NR^{c})N(R^{c})₂, —C(═NR^{c})N(R^{c})₂, hydrogen, (C₁-C₆)alkyl, (C₁-C₆)alkenyl, —C(═O)R^{c}, —C(═O)N(R^{c})₂, —S(═O)₂N(R^{c})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl. (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl- is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{c})₂, —NR^{c}C(═NR^{c})N(R^{c})₂, —C(═NR^{c})N(R^{c})₂, and —OP(═O)(OH)₂;
X¹ is CR³ or N;
X² is CR⁴ or N;
R³ is hydrogen, halo, —OH, —NO₂, —CN, (C₁-C₆)alkoxy, (C₁-C₆)alkyl, (C₁-C₆)alkenyl, —C(═O)R^{d}, —C(═O)N(R^{d})₂, —S(═O)₂N(R^{d})₂, —NR^{d}C(═NR^{d})N(R^{d})₂, —C(═NR^{d})N(R^{d})₂, or —N(R^{d})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{d})₂, —NR^{d}C(═NR^{d})N(R^{d})₂, —C(═NR^{d})N(R^{d})₂, and —OP(═O)(OH)₂;
R⁴ is hydrogen, halo, —OH, —NO₂, —CN, (C₁-C₆)alkoxy, (C₁-C₆)alkyl, (C₁-C₆)alkenyl, —C(═O)R^{e}, —C(═O)N(R^{e})₂, —S(═O)₂N(R^{e})₂, —NR^{e}C(═NR^{e})N(R^{e})₂, —C(═NR^{e})N(R^{e})₂, or —N(R^{e})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{e})₂, —NR^{e}C(═NR^{e})N(R^{e})₂, —C(═NR^{e})N(R^{e})₂, and —OP(═O)(OH)₂;
Y¹ is NR^{5a}, Y² is N or CR^{6b}, Y³ is N or CR^{7b}, the dashed bond between Y¹ and Y² is a single bond, and the dashed bond between Y² and Y³ is a double bond; or
Y³ is NR^{7a}, Y¹ is N or CR^{5b}, Y² is N or CR^{6b}, the dashed bond between Y¹ and Y² is a double bond, and the dashed bond between Y² and Y³ is a single bond;
R^{5a} and R^{7a} are each independently (C₁-C₁₀)alkyl, (C₁-C₁₀)alkenyl, —W—(C₃-C₇)carbocyclyl, —W-aryl, W-heterocyclyl, -or W-heteroaryl, wherein the (C₁-C₁₀)alkyl, (C₁-C₁₀)alkenyl, —W—(C₃-C₇)carbocyclyl, —W-aryl, W-heterocyclyl, —W-heteroaryl, is optionally substituted with one or more (e.g., 1, 2, 3, 4, or 5) groups independently selected from Q;
R^{5b}, R^{6b}, and R^{7b} are each independently hydrogen, halo, —OH, —NO₂, —CN, (C₁-C₆)alkoxy, (C₁-C₆)alkyl, (C₁-C₆)alkenyl, —C(═O)R^{f}, —C(═O)N(R^{f})₂, —S(═O)₂N(R^{f})₂, —NR^{f}C(═NR^{f})N(R^{f})₂, —C(═NR^{f})N(R^{f})₂, or —N(R^{f})₂, wherein the (C₁-C₆)alkoxy, (C₁-C₅)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{f})₂, —NR^{f}C(═NR^{f})N(R^{f})₂, —C(═NR^{f})N(R^{f})₂, and —OP(═O)(OH)₂;
W is absent or (C₁-C₁₀)alkyl, wherein the (C₁-C₁₀)alkyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—;
each Q is independently halo, —OH, —NO₂, —CN, (C₁-C₆)alkyl, (C₁-C₆)haloalkyl, (C₁-C₆)alkoxy, (C₁-C₆)haloalkoxy, C(═O)R^{q}, —C(═O)N(R^{q})₂, —N(R^{q})₂, —S(═O)₂N(R^{a})₂, aryl, —O-aryl, heteroaryl, or —O-heteroaryl, wherein the (C₁-C₆)alkyl, (C₁-C₆)haloalkyl, (C₁-C₆)alkoxy, (C₁-C₆)haloalkoxy, aryl, -Oaryl, heteroaryl, or -Oheteroaryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q}, —C(═O)O(R^{q}), —C(═O)N(R^{q})₂, —S(═O)₂N(R^{q})₂, aryl and heteroaryl, wherein the aryl or heteroaryl is optionally substituted with one or more (e.g., 1, 2, 3, 4, or 5) Q¹;
each Q¹ is independently halo, —OH, —NO₂, —CN, (C₁-C₄)alkyl, (C₁-C₄)haloalkyl, (C₁-C₄)alkoxy, or (C₁-C₄)haloalkoxy;
each R^{a}, R^{b}, R^{ab}, R^{c}, R^{d}, R^{e}, and R^{f}, is independently hydrogen, (C₁-C₆)alkyl, (C₁-C₆)alkenyl, (C₁-C₆)haloalkyl, (C₃-C₇)carbocyclyl, or aryl;
each R^{w} is independently hydrogen or (C₁-C₆)alkyl;
R^{q} is hydrogen, (C₁-C₆)alkyl, (C₁-C₆)alkenyl, (C₁-C₆)haloalkyl (C₃-C₇)carbocyclyl or aryl, wherein the aryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q1}, —C(═O)O(R^{q1}), —C(═O)N(R^{q1})₂, and —S(═O)₂N(R^{q1})₂; and
each R^{q1} is independently hydrogen, (C₁-C₆)alkyl, (C₁-C₆)alkenyl, (C₁-C₆)haloalkyl, (C₃-C₇)carbocyclyl, or aryl;
or a salt thereof.

One embodiment provides a pharmaceutical composition comprising a compound of formula I or a pharmaceutically acceptable salt thereof as described herein, and a pharmaceutically acceptable vehicle.

One embodiment provides pharmaceutical composition comprising a compound of formula I or a pharmaceutically acceptable salt thereof as described herein, one or more (e.g., 1 or 2) antibacterial agents and a pharmaceutically acceptable vehicle.

One embodiment provides a method of treating or preventing a bacterial infection in an animal (e.g., a mammal such as a human) comprising administering to the animal a compound of formula I or a pharmaceutically acceptable salt thereof as described herein.

One embodiment provides a method of treating or preventing a bacterial infection in an animal (e.g., a mammal such as a human) comprising administering to the animal a compound of formula I or a pharmaceutically acceptable salt thereof as described herein and one or more (e.g., 1 or 2) antibacterial agents.

One embodiment provides a method of treating or preventing a bacterial infection in an animal (e.g., a mammal such as a human) comprising administering to the animal in need thereof a compound of formula I or a pharmaceutically acceptable salt thereof as described herein.

One embodiment provides a method of treating or preventing a bacterial infection in an animal (e.g., a mammal such as a human) infected with bacteria comprising administering to the animal a compound of formula I or a pharmaceutically acceptable salt thereof as described to herein.

One embodiment provides a compound of formula I or a pharmaceutically acceptable salt thereof as described herein for use in medical treatment.

One embodiment provides a compound of formula I or a pharmaceutically acceptable salt thereof as described herein for the prophylactic or therapeutic treatment of a bacterial infection.

One embodiment provides the use of a compound of formula I or a pharmaceutically acceptable salt thereof as described herein for the preparation of a medicament for the prophylactic or therapeutic treatment of a bacterial infection in an animal.

One embodiment provides the use of a compound of formula I or a pharmaceutically acceptable salt thereof as described herein for the preparation of a medicament for treating a bacterial infection in an animal (e.g., a mammal such as a human).

One embodiment provides processes and intermediates disclosed herein that are useful for preparing compounds of formula I or salts thereof.

One embodiment provides a prodrug of a compound of formula I.

### DETAILED DESCRIPTION

The following definitions are used, unless otherwise described: halo or halogen is fluoro, chloro, bromo, or iodo. Alkyl, alkenyl and alkoxy, etc. denote both straight and branched groups but reference to an individual radical such as propyl embraces only the straight chain radical (a branched chain isomer such as isopropyl being specifically referred to).

As used herein, the term “(Cₐ-C_{b})alkyl” wherein a and b are integers refers to a straight or branched chain hydrocarbon alkyl radical having from a to b carbon atoms. Thus, when a is 1 and b is 6, for example, the term includes methyl, ethyl, n-propyl, isopropyl, n-butyl, isobutyl, sec-butyl, t-butyl, n-pentyl and n-hexyl.

The term “alkenyl” as used herein refers to alkyl that contains one or more double bonds.

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like. In one embodiment aryl is phenyl or naphthyl.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1, 2, 3, 4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. In one embodiment a monocyclic or bicyclic heteroaryl has 5 to 10 ring atoms comprising 1 to 9 carbon atoms and 1 to 4 heteroatoms wherein at least one ring containing at least one heteroatom is aromatic. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “heterocyclyl” or “heterocycle” as used herein refers to a single saturated or partially unsaturated ring containing at least one heteroatom or a multiple ring system wherein at least one ring is saturated or partially unsaturated and contains at least one heteroatom. The term includes single saturated or partially unsaturated rings (e.g., 3, 4, 5, 6 or 7-membered rings) from about 1 to 6 carbon atoms and from about 1 to 3 (e.g., 1, 2 or 3) heteroatoms selected from the group consisting of oxygen, nitrogen and sulfur in the ring. In one embodiment a monocyclic or bicyclic heterocyclyl has 5 to 10 ring atoms comprising 1 to 9 carbon atoms and 1 to 4 heteroatoms (e.g., 1, 2, 3, or 4) selected from the group consisting of oxygen, nitrogen and sulfur in the ring wherein the at least one ring is saturated or partially unsaturated and includes at least one heteroatom. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term “haloalkyl” includes an alkyl group as defined herein that is substituted with one or more (e.g., 1, 2, 3, or 4) halo groups. One specific halo alkyl is a “(C₁-C₆)haloalkyl”.

The term “alkoxy” refers to an —O-alkyl group.

The term cycloalkyl, carbocycle, or carbocyclyl includes saturated and partially unsaturated carbocyclic ring systems. In one embodiment the carbocyclyl is a monocyclic carbocyclic ring. Such carbocyclyls include “(C₃-C₇)carbocyclyl” and “(C₃-C₈)cycloalkyl”. Examples of carbocyclyls include without limitation cyclopropane, cyclobutane, cyclobutene, cyclopentane, cyclopentene, cyclohexane, cyclohexene, cycloheptane and cycloheptene.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fiuoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

It is understood that the embodiments provided below are for compounds of formula I and all sub-formulas thereof (e.g., formulas Ia, lb, Ic, Id, Ie, If, Ig, Ih). It is to be understood the two or more embodiments may be combined.

In one embodiment X¹ is CR³.

In one embodiment X² is CR⁴.

In one embodiment X² is N.

In one embodiment a compound of formula I is a compound of formula Ia:

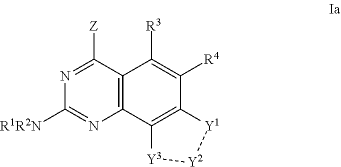

or a salt thereof.

In one embodiment a compound of formula I is a compound of formula Ib:

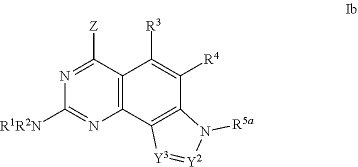

or a salt thereof.

In one embodiment a compound of formula I is a compound of formula Ic:

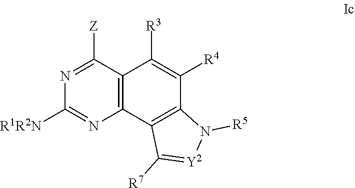

or a salt thereof.

In one embodiment Y is CR^{7b}.

In one embodiment Y² is CR^{6b}.

In one embodiment a compound of formula I is a compound of formula Id:

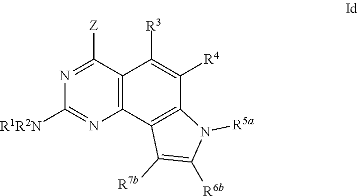

or a salt thereof.

In one embodiment a compound of formula I is a compound of formula Ie:

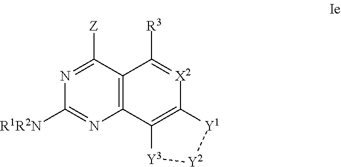

or a salt thereof.

In one embodiment a compound of formula I is a compound of formula If:

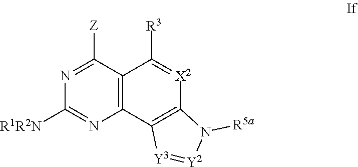

or a salt thereof.

In one embodiment a compound of formula I is a compound of formula Ig:

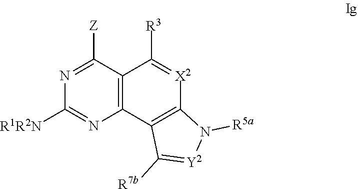

or a salt thereof.

In one embodiment the compound of formula I is a compound of formula Ih:

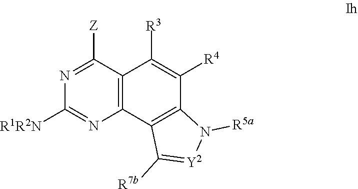

or a salt thereof.

In one embodiment a compound of formula I is a compound of formula Ii:

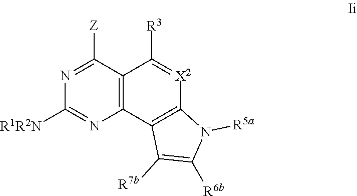

or a salt thereof.

In one embodiment R³ is hydrogen.

In one embodiment R³ is hydrogen or halo.

In one embodiment R⁴ is hydrogen.

In one embodiment R⁴ is hydrogen or halo

In one embodiment R¹ is hydrogen, (C₁-C₆)alkyl, or aryl(C₁-C₆)alkyl-, wherein the (C₁-C₆)alkyl or aryl(C₁-C₆)alkyl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, phenyl, and —OP(═O)(OH)₂; wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy.

In one embodiment R¹R¹ is hydrogen, (C₁-C₆)alkyl, or aryl(C₁-C₆)alkyl-, wherein the (C₁-C₆)alkyl or aryl(C₁-C₆)alkyl is optionally substituted with one or more (e.g., 1, 2, 3, 4, or 5) groups independently selected from the group consisting of halo and phenyl, wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy.

In one embodiment R¹ is hydrogen.

In one embodiment R² is hydrogen.

In one embodiment Z is —N(R^{c})₂.

In one embodiment Z is —NH₂.

In one embodiment R^{7b} is hydrogen.

In one embodiment R^{6b} is hydrogen.

In one embodiment R^{7b} is hydrogen or (C₁-C₆)alkyl.

In one embodiment R^{6b} is hydrogen or (C₁-C₆)alkyl.

In one embodiment R^{5a} is (C₁-C₁₀)alkenyl, —W—(C₃-C⁷)carbocyclyl, —W-aryl, or —W-heteroaryl, wherein the (C₁-C₁₀)alkenyl, —W—(C₃-C₇)carbocyclyl, —W-aryl, —W-heteroaryl, is optionally substituted with one or more groups independently selected from Q.

In one embodiment R^{5a} is (C₁-C₁₀)alkyl, (C₂-C₁₀)alkenyl, —W—(C₃-C₇)carbocyclyl, —W-aryl, or —W-heteroaryl, wherein the C₁-C₁₀)alkyl, (C₂-C₁₀)alkenyl, —W—(C₃-C₇)carbocyclyl, —W-aryl, or —W-heteroaryl, is optionally substituted with one or more groups independently selected from Q.

In one embodiment R^{5a} is (C₂-C₁₀)alkenyl, —W—(C₃-C₇)carbocyclyl, —W-aryl, or —W-heteroaryl, wherein the (C₂-C₁₀)alkenyl, —W—(C₃-C₇)carbocyclyl, —W-aryl, or —W-heteroaryl, is optionally substituted with one or more groups independently selected from Q.

In one embodiment W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—.

In one embodiment R^{5a} is (C₂-C₆)alkenyl, —W—(C₃-C₇)carbocyclyl, —W-aryl, or —W-heteroaryl, wherein the (C₂-C₆)alkenyl, —W—(C₃-C₇)carbocyclyl, —W-aryl, —W-heteroaryl, is optionally substituted with one or more groups independently selected from Q.

In one embodiment R^{5a} is (C₁-C₆)alkyl, (C₂-C₆)alkenyl, —W—(C₃-C₇)carbocyclyl, —W-aryl, or —W-heteroaryl, wherein the C₁-C₆)alkyl, (C₂-C₆)alkenyl, —W—(C₃-C₇)carbocyclyl, —W-aryl, or —W-heteroaryl, is optionally substituted with one or more groups independently selected from Q.

In one embodiment R^{5a} is (C₂-C₆)alkenyl, —W—(C₃-C₇)carbocyclyl, —W-aryl, or —W-heteroaryl, wherein the (C₂-C₆)alkenyl, —W—(C₃-C₇)carbocyclyl, —W-aryl, or —W-heteroaryl, is optionally substituted with one or more groups independently selected from Q.

In one embodiment W is absent, (C₁-C₆)alkyl, or (C₂-C₆)alkynyl wherein the (C₁-C₆)alkyl or (C₂-C₆)alkynyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—.

In one embodiment W is absent or (C₁-C₄)alkyl, wherein the (C₁-C₄)alkyl is optionally substituted with one or more oxo (═O).

In one embodiment W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O).

In one embodiment W is absent, —CH₂—, —C(═O)—, or propyne.

In one embodiment W is absent, —CH₂—, or —C(═O)—.

In one embodiment R^{5a} is:

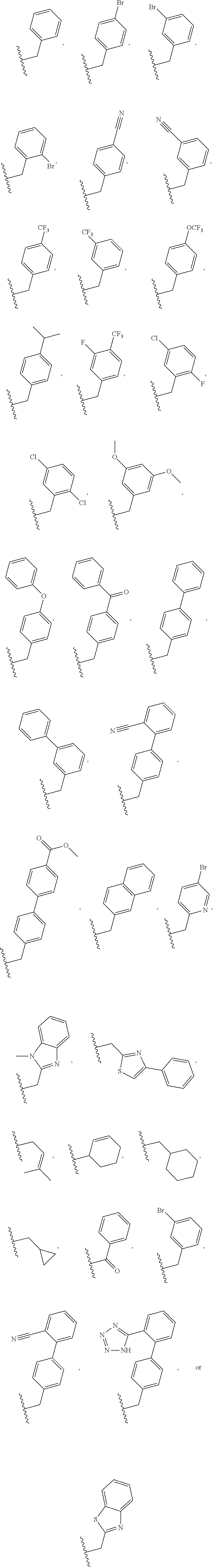

In one embodiment R^{5a} is:

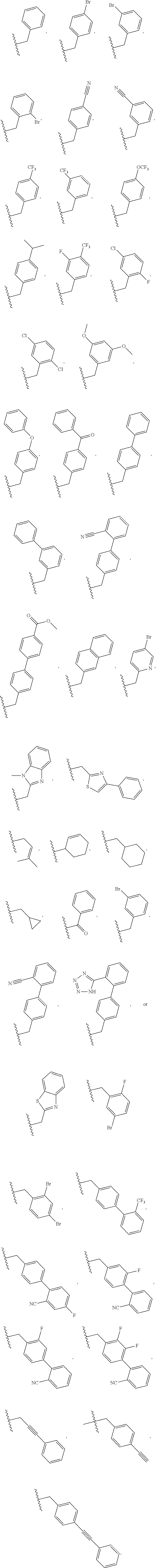

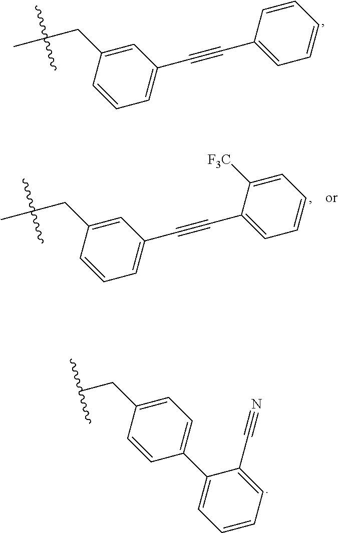 [SMILES: *Cc1cccc(C#Cc2ccccc2)c1 ; *Cc1cccc(C#Cc2ccccc2C(F)(F)F)c1 ; *Cc1ccc(-c2ccccc2C#N)cc1]

One embodiment provides the compound:

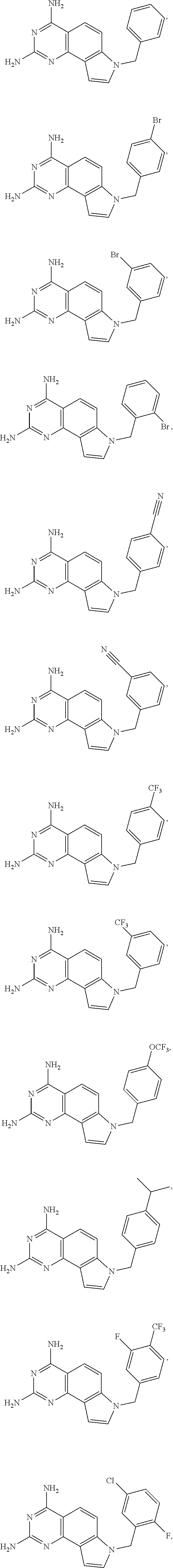

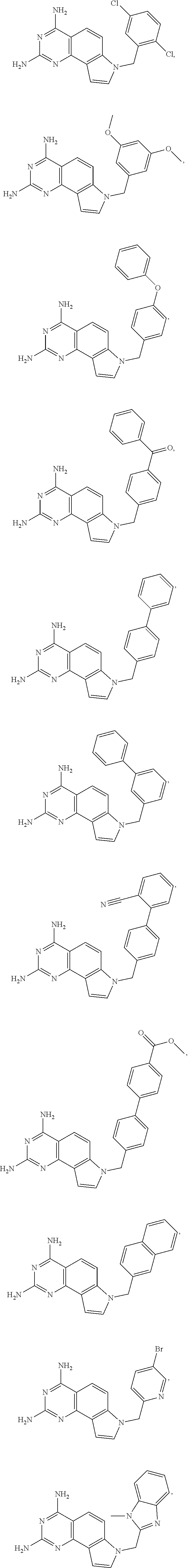

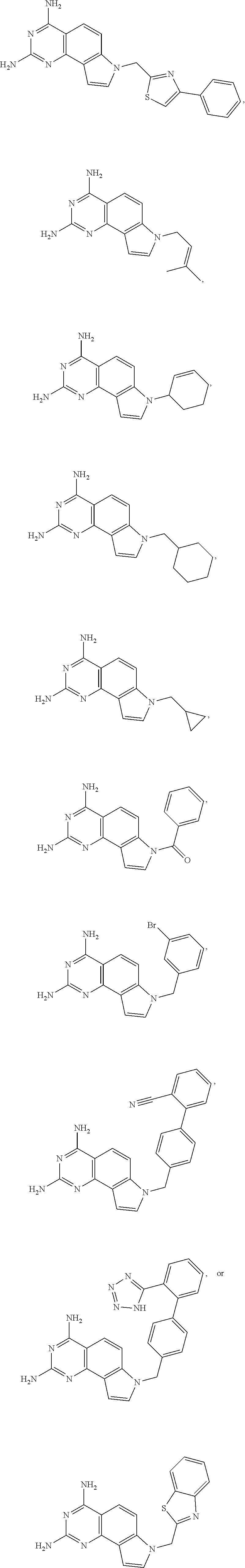

or a salt thereof.

One embodiment provides the compound:

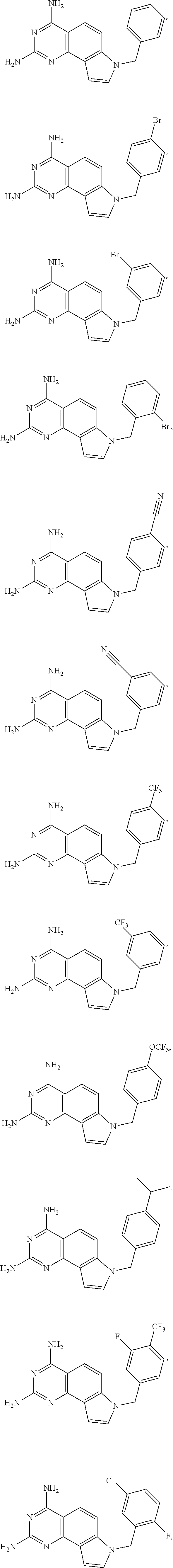

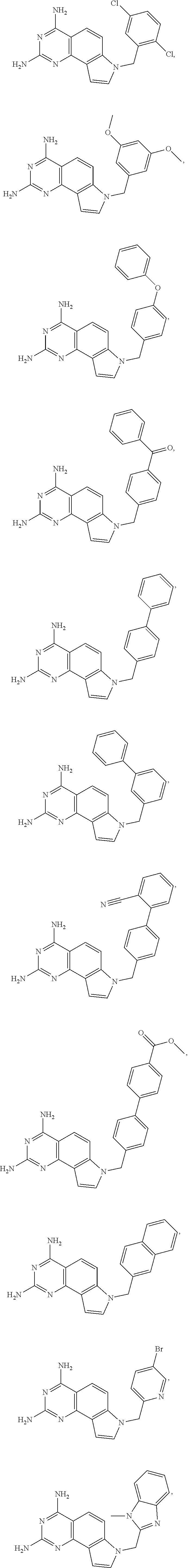

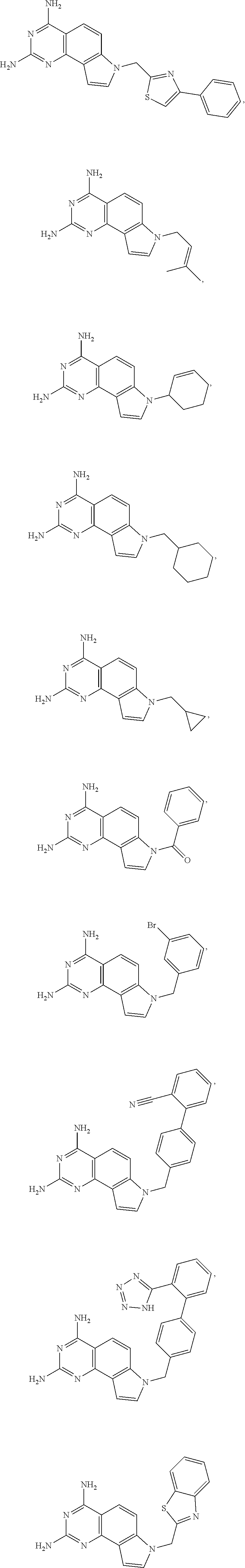

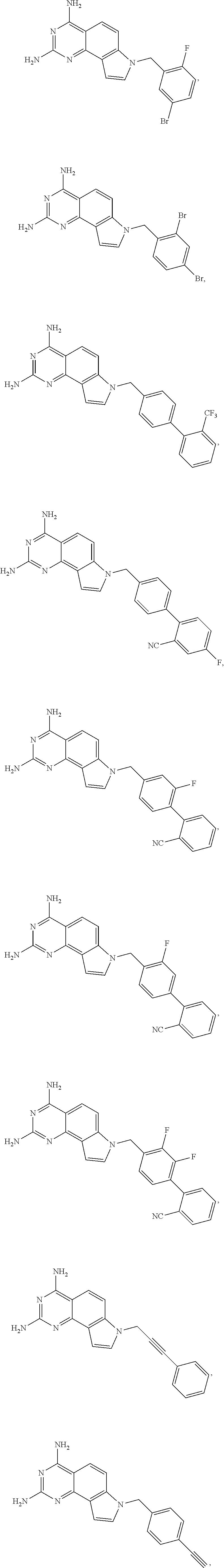

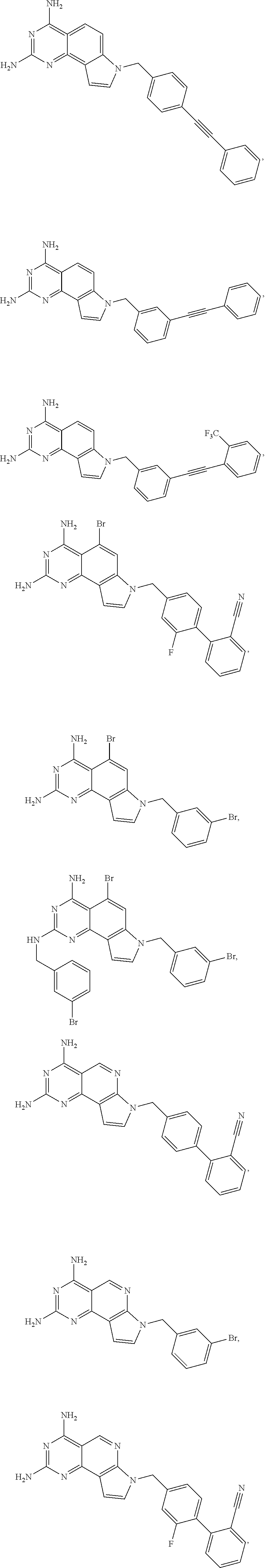

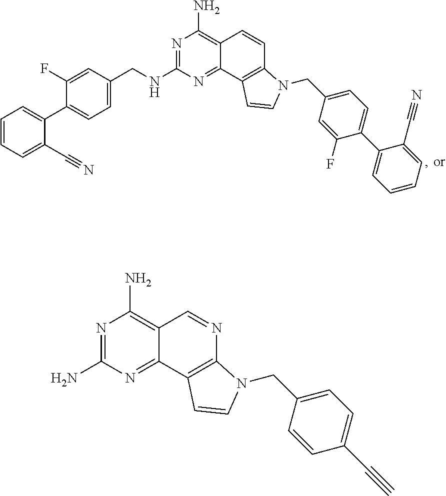 [SMILES: N#Cc1ccccc1-c1ccc(CNc2nc(N)c3ccc4c(ccn4Cc4ccc(-c5ccccc5C#N)c(F)c4)c3n2)cc1F ; C#Cc1ccc(Cn2ccc3c4nc(N)nc(N)c4cnc32)cc1]

or a salt thereof.

Generally, compounds of formula I as well as synthetic intermediates that can be used for preparing compounds of formula I can be prepared as illustrated in the following general schemes. It is understood that variable groups shown below (e.g., R³, R⁴, R^{5a}, R^{6b}, R^{7b}) can represent the final corresponding groups present in a compound of formula I or that these groups can represent groups that can be converted to the final corresponding groups present in a compound of formula I at a convenient point in a synthetic sequence. For example, the variable groups can contain one or more protecting groups that can be removed at a convenient point in a synthetic sequence to provide the final corresponding groups in the compound of formula I. Schemes 1-3 illustrates a general method for the preparation of compounds of formula I.

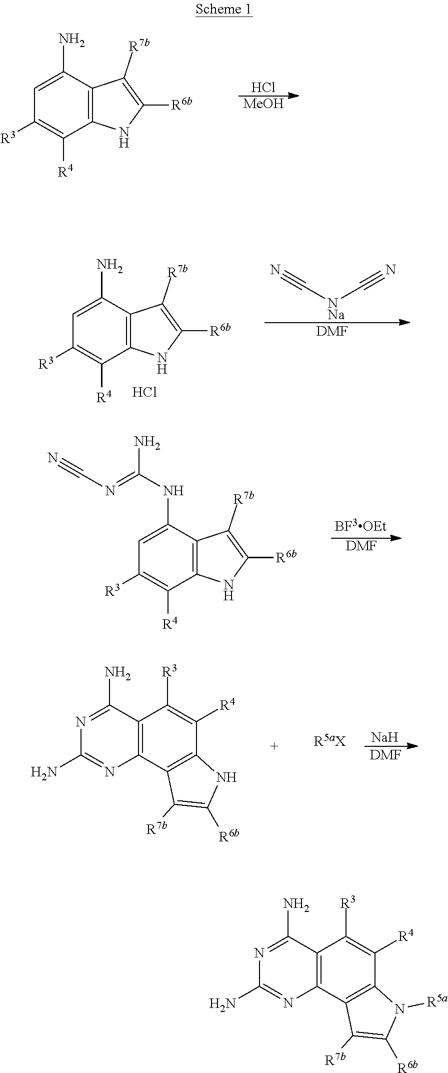

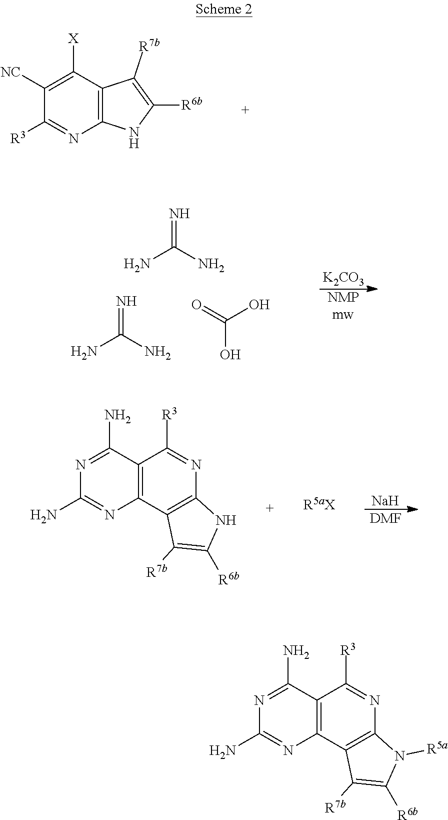

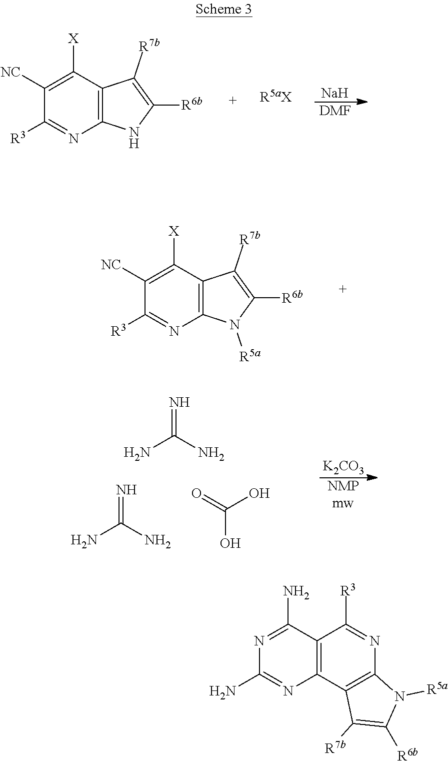

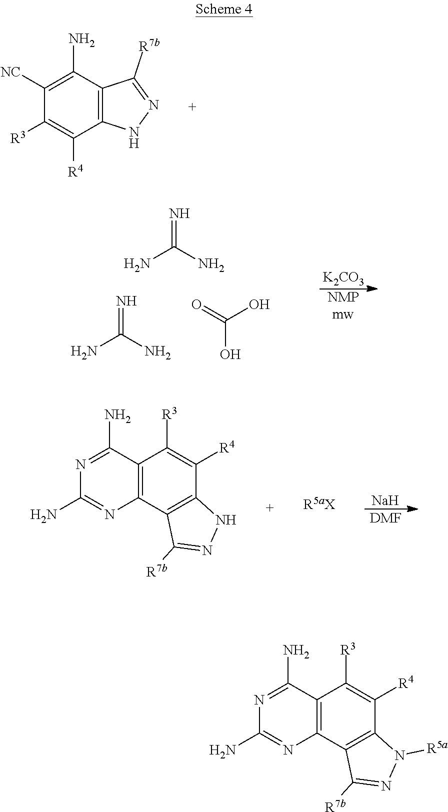

In one embodiment the bacterial infection being treated is a Gram-negative bacterial strain infection. In one embodiment the Gram-negative bacterial strain is selected from the group consisting of Acinetobacter baumannii, Acinetobacter calcoaceticus, Acinetobacter haemolyticus, Acinetobacter lwoffi, Actinobacillus actinomycetemcomitans, Aeromonas hydrophilia, Aggregatibacter actinomycetemcomitans, Agrobacterium tumefaciens, Bacteroides distasonis, Bacteroides eggerthii, Bacteroides forsythus, Bacteroides fragilis, Bacteroides ovalus, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Branhamella catarrhalis, Burkholderia cepacia, Campylobacter coli, Campylobacter fetus, Campylobacter jejuni, Caulobacter crescentus, Chlamydia trachomatis, Citrobacter diversus, Citrobacter freundii, Enterobacter aerogenes, Enterobacter asburiae, Enterobacter cloacae, Enterobacter sakazakii, Escherchia coli, Francisella tularensis, Fusobacterium nucleatum, Gardnerella vaginalis, Haemophilus ducreyi, Haemophilus haemolyticus, Haemophilus influenzae, Haemophilus parahaemolyticus, Haemophilus parainfluenzae, Helicobacter pylori, Kingella denitrificans, Kingella indologenes, Kingella kingae, Kingella oralis, Klebsiella oxytoca, Klebsiella pneumoniae, Klebsiella rhinoscleromatis, Legionella pneumophila, Listeria monocytogenes, Moraxella bovis, Moraxella catarrhalis, Moraxella lacunata, Morganella morganii, Neisseria gonorrhoeae, Neisseria meningitidis, Pantoea agglomerans, Pasteurella canis, Pasteurella haemolytica, Pasteurella multocida, Pasteurella tularensis, Porphyromonas gingivalis, Proteus mirabilis, Proteus vulgaris, Providencia alcalifaciens, Providencia rettgeri, Providencia stuartii, Pseudomonas acidovorans, Pseudomonas aeruginosa, Pseudomonas alcaligenes, Pseudomonas fluorescens, Pseudomonas putida. Salmonella enteriditis, Salmonella paratyphi, Salmonella typhi, Salmonella typhimurium, Serratia marcescens, Shigella dysenteriae, Shigella jlexneri, Shigella sonnei, Stenotrophomonas maltophilla, Veillonella parvula, Vibrio cholerae, Vibrio parahaemolyticus, Yersinia enterocolitica, Yersinia intermedia, Yersinia pestis and Yersinia pseudotuberculosis.

In one embodiment the bacterial infection being treated is a Gram-positive bacterial strain infection. In one embodiment the Gram-positive bacterial strain is selected from the group consisting of Actinomyces naeslundii, Actinomyces viscosus. Bacillus anthracis, Bacillus cereus, Bacillus subtilis, Clostridium difficile, Corynebacterium diphtheriae, Corynebacterium ulcerans, Enterococcus faecalis. Enterococcus faecium. Micrococcus luteus, Mycobacterium avium. Mycobacterium intracellulare, Mycobacterium leprae, Mycobacterium tuberculosis, Propionibacterium acnes, Staphylococcus aureus, Staphylococcus epidermidis, Staphylococcus haemolyticus, Staphylococcus hominis, Staphylococcus hyicus, Staphylococcus intermedius, Staphylococcus saccharolyticus, Staphylococcus saprophyticus, Streptococcus agalactiae, Streptococcus mutans, Streptococcus pneumoniae, Streptococcus pyogenes, Streptococcus salivarius and Streptococcus sanguis.

The compositions can, if desired, also contain other active therapeutic agents, such as a narcotic, a non-steroid anti-inflammatory drug (NSAID), an analgesic, an anesthetic, a sedative, a local anesthetic, a neuromuscular blocker, an anti-cancer, an antimicrobial (for example, an aminoglycoside, an antifungal, an antiparasitic, an antiviral, a carbapenem, a cephalosporin (e.g., cefepime), a fluoroquinolone, a macrolide, a penicillin, a sulfonamide, a tetracycline, another antimicrobial), an anti-psoriatic, a corticosteriod, an anabolic steroid, a diabetes-related agent, a mineral, a nutritional, a thyroid agent, a vitamin, a calcium-related hormone, an antidiarrheal, an anti-tussive, an anti-emetic, an anti-ulcer, a laxative, an anticoagulant, an erythropoietin (for example, epoetin alpha), a filgrastim (for example, G-CSF, Neupogen), a sargramostim (GM-CSF, Leukine), an immunization, an immunoglobulin, an immunosuppressive (for example, basiliximab, cyclosporine, daclizumab), a growth hormone, a hormone replacement drug, an estrogen receptor modulator, a mydriatic, a cycloplegic, an alkylating agent, an anti-metabolite, a mitotic inhibitor, a radiopharmaceutical, an anti-depressant, an anti-manic agent, an anti-psychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog thereof, dornase alpha (Pulmozyme), a cytokine, or any combination thereof.

In one embodiment the antibacterial agent is selected from quinolones, tetracyclines, glycopeptides, aminoglycosides, β-lactams, rifamycins, macrolides, ketolides, oxazolidinones, coumermycins, and chloramphenicol.

It will be appreciated that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound (or composition thereof) may be at least 51% the absolute stereoisomer depicted. In another embodiment, the compound (or composition thereof) may be at least 60% the absolute stereoisomer depicted. In another embodiment, the compound (or composition thereof) may be at least 80% the absolute stereoisomer depicted. In another embodiment, the compound (or composition thereof) may be at least 90% the absolute stereoisomer depicted. In another embodiment, the compound (or composition thereof) may be at least 95 the absolute stereoisomer depicted. In another embodiment, the compound (or composition thereof) may be at least 99% the absolute stereoisomer depicted.

It will also be appreciated by those skilled in the art that certain compounds of the invention can exist in more than one tautomeric form. For example, a substituent of formula —NH—C(═O)H in a compound of formula (I) could exist in tautomeric form as —N═C(OH)H. The present invention encompasses all tautomeric forms of a compound of formula I as well as mixtures thereof that can exist in equilibrium with non-charged and charged entities depending upon pH, which possess the useful properties described herein

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, fumarate, benzoate, ascorbate, α-ketoglutarate, and α-glycerophosphate. Suitable inorganic salts may also be formed, including hydrochloride, sulfate, nitrate, bicarbonate, and carbonate salts. Salts may be obtained using standard procedures well known in the art, for example by reacting a sufficiently basic compound such as an amine with a suitable acid affording the corresponding anion. Alkali metal (for example, sodium, potassium or lithium) or alkaline earth metal (for example calcium) salts of carboxylic acids can also be made.

Pharmaceutically suitable counterions include pharmaceutically suitable cations and pharmaceutically suitable anions that are well known in the art. Examples of pharmaceutically suitable anions include, but are not limited to those described above (e.g. physiologically acceptable anions) including Cl⁻, Br⁻, I⁻, CH₃SO₃⁻, H₂PO₄⁻, CF₃SO₃⁻, p-CH₃C₆H₄SO₃⁻, citrate, tartrate, phosphate, malate, fumarate, formate, or acetate.

It will be appreciated by those skilled in the art that a compound of the invention comprising a counterion can be converted to a compound of the invention comprising a different counterion. Such a conversion can be accomplished using a variety of well-known techniques and materials including but not limited to ion exchange resins, ion exchange chromatography and selective crystallization.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes. For oral administration, the compounds can be formulated as a solid dosage form with or without an enteric coating.

Thus, the present compounds may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The active compound may also be administered intravenously or intramuscularly by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vilro activity, and in vivo activity in animal models. Methods for the extrapolation of effective dosages in mice, and other animals, to humans are known to the art, for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 1 to about 500 mg/kg, e.g., from about 5 to about 400 mg/kg of body weight per day, such as 1 to about 250 mg per kilogram body weight of the recipient per day.

The compound is conveniently formulated in unit dosage form; for example, containing 5 to 500 mg, 10 to 400 mg, or 5 to 100 mg of active ingredient per unit dosage form. In one embodiment, the invention provides a composition comprising a compound of the invention formulated in such a unit dosage form. The desired dose may conveniently be presented in a single dose or as divided doses administered at appropriate intervals, for example, as two, three, four or more sub-doses per day. The sub-dose itself may be further divided. e.g., into a number of discrete loosely spaced administrations.

Co-administration of a compound disclosed herein with one or more other active therapeutic agents (e.g., antibacterial agents) generally refers to simultaneous or sequential administration of a compound disclosed herein and one or more other active therapeutic agents, such that therapeutically effective amounts of disclosed herein and one or more other active therapeutic agents are both present in the body of the patient.

The ability of a compound disclosed herein to inhibit a N. gonorrhoeae growth is shown in Table 1 and can be determined using a method as described in Example 34.

TABLE 1

| | | MIC |
|---|---|---|
| Example | Structure | (μg/mL)¹ |
| 1 | 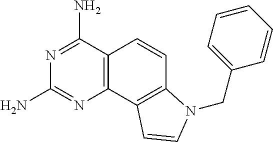 [SMILES: Nc1nc(N)c2ccc3c(ccn3Cc3ccccc3)c2n1] | 8 |
| 2 | 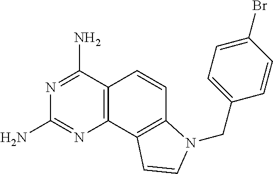 [SMILES: Nc1nc(N)c2ccc3c(ccn3Cc3ccc(Br)cc3)c2n1] | 2 |
| 3 | 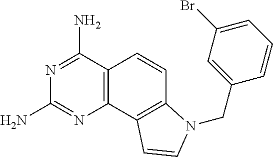 [SMILES: Nc1nc(N)c2ccc3c(ccn3Cc3cccc(Br)c3)c2n1] | 0.5 |
| 4 | 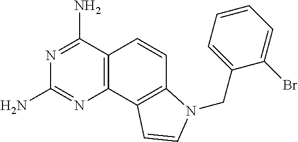 [SMILES: Nc1nc(N)c2ccc3c(ccn3Cc3ccccc3Br)c2n1] | 2 |
| 5 | 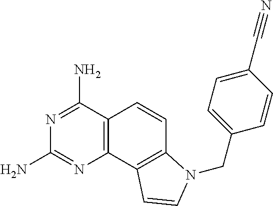 [SMILES: N#Cc1ccc(Cn2ccc3c4nc(N)nc(N)c4ccc32)cc1] | 16 |
| 6 | 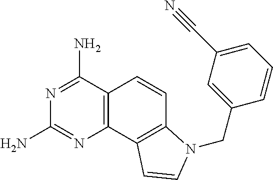 [SMILES: N#Cc1cccc(Cn2ccc3c4nc(N)nc(N)c4ccc32)c1] | 4 |
| 7 | 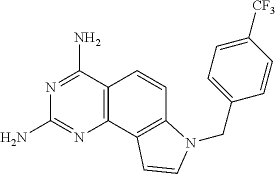 [SMILES: Nc1nc(N)c2ccc3c(ccn3Cc3ccc(C(F)(F)F)cc3)c2n1] | 2 |
| 8 | 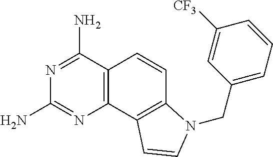 [SMILES: Nc1nc(N)c2ccc3c(ccn3Cc3cccc(C(F)(F)F)c3)c2n1] | 8 |
| 9 | 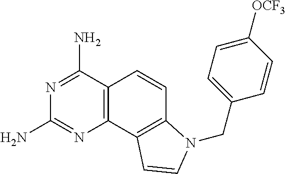 [SMILES: Nc1nc(N)c2ccc3c(ccn3Cc3ccc(OC(F)(F)F)cc3)c2n1] | 16 |
| 10 | 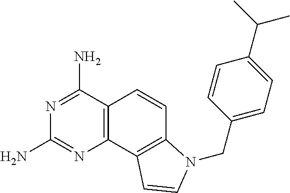 [SMILES: CC(C)c1ccc(Cn2ccc3c4nc(N)nc(N)c4ccc32)cc1] | 4 |
| 11 | 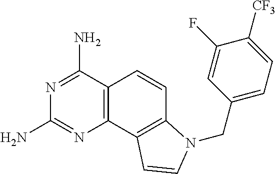 [SMILES: Nc1nc(N)c2ccc3c(ccn3Cc3ccc(C(F)(F)F)c(F)c3)c2n1] | 4 |
| 12 | 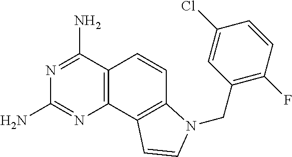 [SMILES: Nc1nc(N)c2ccc3c(ccn3Cc3cc(Cl)ccc3F)c2n1] | 4 |
| 13 | 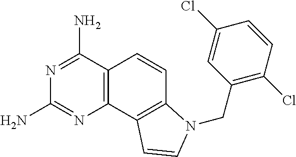 [SMILES: Nc1nc(N)c2ccc3c(ccn3Cc3cc(Cl)ccc3Cl)c2n1] | 16 |
| 14 | 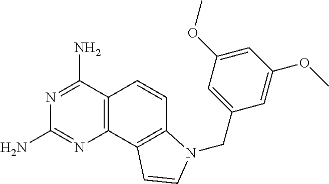 [SMILES: COc1cc(Cn2ccc3c4nc(N)nc(N)c4ccc32)cc(OC)c1] | >64 |
| 15 | 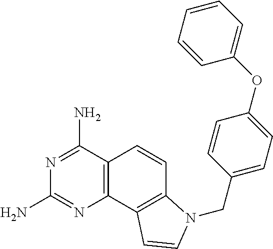 [SMILES: Nc1nc(N)c2ccc3c(ccn3Cc3ccc(Oc4ccccc4)cc3)c2n1] | 8 |
| 16 | 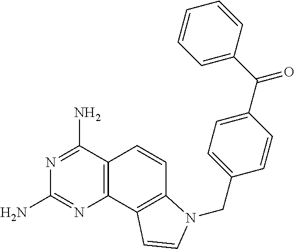 [SMILES: Nc1nc(N)c2ccc3c(ccn3Cc3ccc(C(=O)c4ccccc4)cc3)c2n1] | 4 |
| 17 | 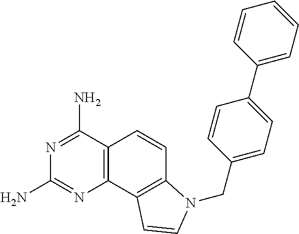 [SMILES: Nc1nc(N)c2ccc3c(ccn3Cc3ccc(-c4ccccc4)cc3)c2n1] | 8 |
| 18 | 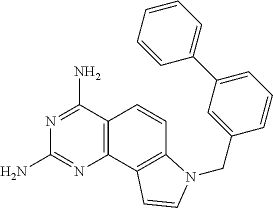 [SMILES: Nc1nc(N)c2ccc3c(ccn3Cc3cccc(-c4ccccc4)c3)c2n1] | 16 |
| 19 | 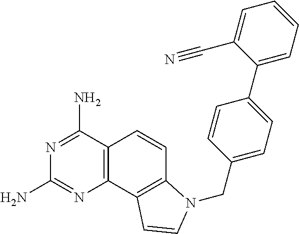 [SMILES: N#Cc1ccccc1-c1ccc(Cn2ccc3c4nc(N)nc(N)c4ccc32)cc1] | 0.25 |
| 20 | 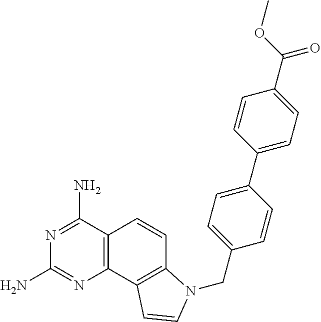 [SMILES: COC(=O)c1ccc(-c2ccc(Cn3ccc4c5nc(N)nc(N)c5ccc43)cc2)cc1] | >32 |
| 21 | 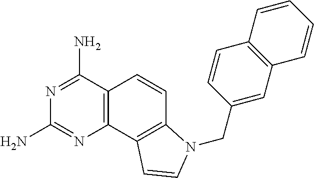 [SMILES: Nc1nc(N)c2ccc3c(ccn3Cc3ccc4ccccc4c3)c2n1] | 2 |
| 22 | 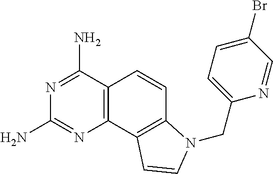 [SMILES: Nc1nc(N)c2ccc3c(ccn3Cc3ccc(Br)cn3)c2n1] | 16 |
| 23 | 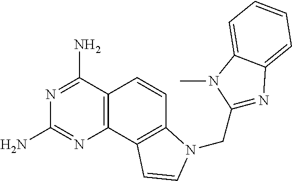 [SMILES: Cn1c(Cn2ccc3c4nc(N)nc(N)c4ccc32)nc2ccccc21] | >64 |
| 24 | 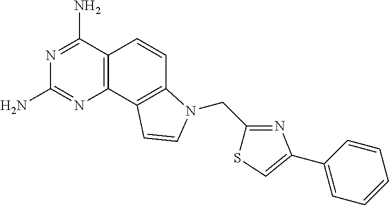 [SMILES: Nc1nc(N)c2ccc3c(ccn3Cc3nc(-c4ccccc4)cs3)c2n1] | 4 |
| 25 | 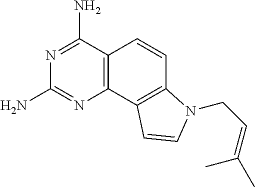 [SMILES: CC(C)=CCn1ccc2c3nc(N)nc(N)c3ccc21] | 4 |
| 26 | 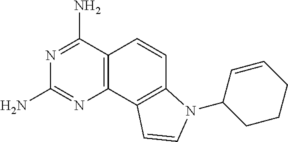 [SMILES: Nc1nc(N)c2ccc3c(ccn3C3C=CCCC3)c2n1] | 4 |
| 27 | 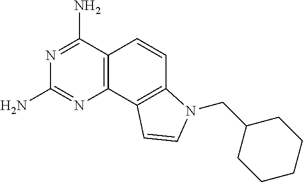 [SMILES: Nc1nc(N)c2ccc3c(ccn3CC3CCCCC3)c2n1] | 4 |
| 28 | 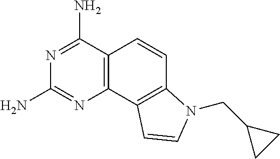 [SMILES: Nc1nc(N)c2ccc3c(ccn3CC3CC3)c2n1] | 8 |
| 29 | 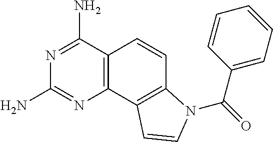 [SMILES: Nc1nc(N)c2ccc3c(ccn3C(=O)c3ccccc3)c2n1] | 32 |
| 30 | 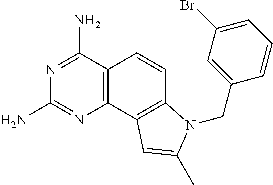 [SMILES: Cc1cc2c3nc(N)nc(N)c3ccc2n1Cc1cccc(Br)c1] | 16 |
| 31 | 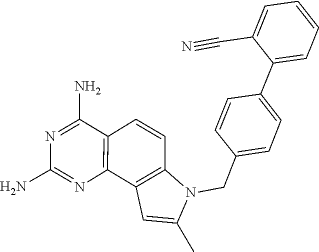 [SMILES: Cc1cc2c3nc(N)nc(N)c3ccc2n1Cc1ccc(-c2ccccc2C#N)cc1] | 1 |
| 32 | 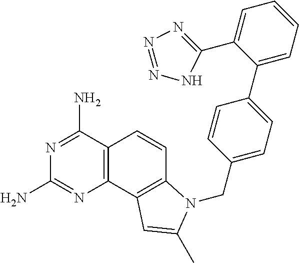 [SMILES: Cc1cc2c3nc(N)nc(N)c3ccc2n1Cc1ccc(-c2ccccc2-c2nnn[nH]2)cc1] | >64 |
| 33 | 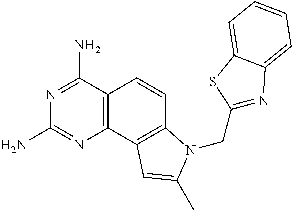 [SMILES: Cc1cc2c3nc(N)nc(N)c3ccc2n1Cc1nc2ccccc2s1] | 8 |
| 34 | 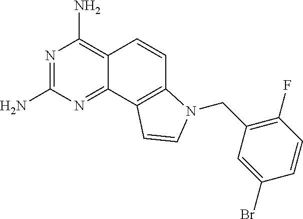 [SMILES: Nc1nc(N)c2ccc3c(ccn3Cc3cc(Br)ccc3F)c2n1] | 16 |
| 35 | 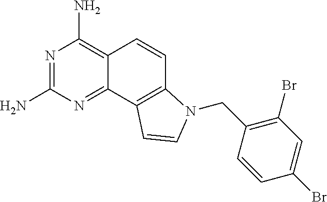 [SMILES: Nc1nc(N)c2ccc3c(ccn3Cc3ccc(Br)cc3Br)c2n1] | 4 |
| 36 | 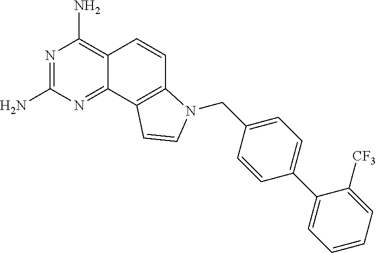 [SMILES: Nc1nc(N)c2ccc3c(ccn3Cc3ccc(-c4ccccc4C(F)(F)F)cc3)c2n1] | 8 |
| 37 | 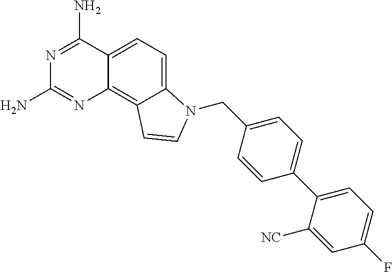 [SMILES: N#Cc1cc(F)ccc1-c1ccc(Cn2ccc3c4nc(N)nc(N)c4ccc32)cc1] | 1 |
| 38 | 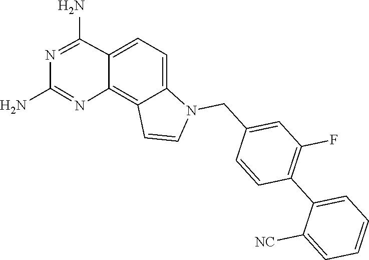 [SMILES: N#Cc1ccccc1-c1ccc(Cn2ccc3c4nc(N)nc(N)c4ccc32)cc1F] | 0.125 |
| 39 | 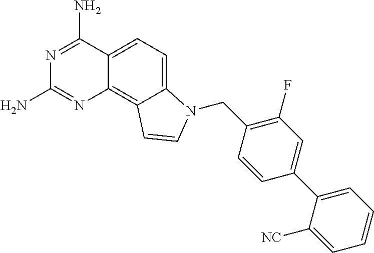 [SMILES: N#Cc1ccccc1-c1ccc(Cn2ccc3c4nc(N)nc(N)c4ccc32)c(F)c1] | ND |
| 40 | 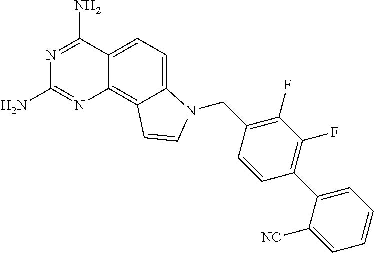 [SMILES: N#Cc1ccccc1-c1ccc(Cn2ccc3c4nc(N)nc(N)c4ccc32)c(F)c1F] | ND |
| 41 | 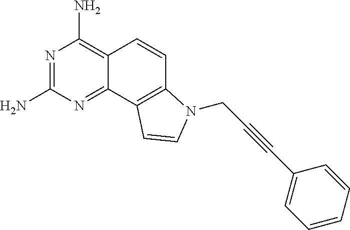 [SMILES: Nc1nc(N)c2ccc3c(ccn3CC#Cc3ccccc3)c2n1] | 8 |
| 42 | 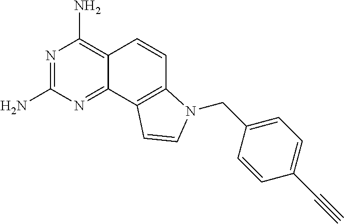 [SMILES: C#Cc1ccc(Cn2ccc3c4nc(N)nc(N)c4ccc32)cc1] | 8 |
| 43 | 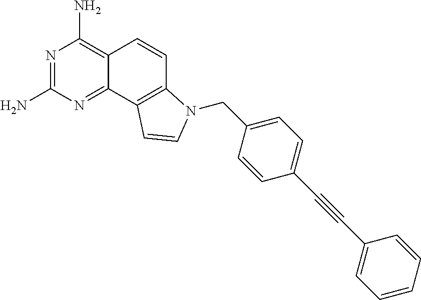 [SMILES: Nc1nc(N)c2ccc3c(ccn3Cc3ccc(C#Cc4ccccc4)cc3)c2n1] | >64 |
| 44 | 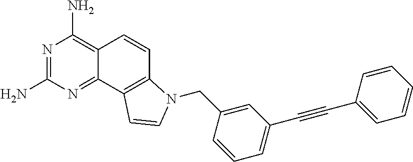 [SMILES: Nc1nc(N)c2ccc3c(ccn3Cc3cccc(C#Cc4ccccc4)c3)c2n1] | ND |
| 45 | 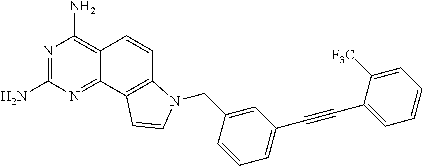 [SMILES: Nc1nc(N)c2ccc3c(ccn3Cc3cccc(C#Cc4ccccc4C(F)(F)F)c3)c2n1] | ND |
| 46 | 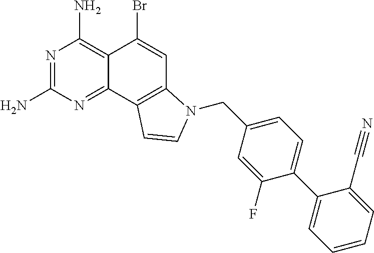 [SMILES: N#Cc1ccccc1-c1ccc(Cn2ccc3c4nc(N)nc(N)c4c(Br)cc32)cc1F] | ND |
| 47 | 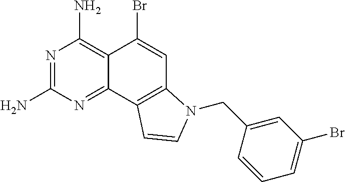 [SMILES: Nc1nc(N)c2c(Br)cc3c(ccn3Cc3cccc(Br)c3)c2n1] | ND |
| 48 | 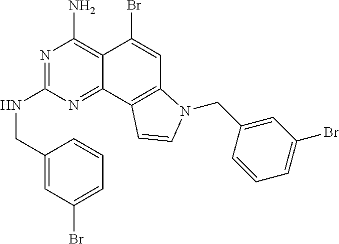 [SMILES: Nc1nc(NCc2cccc(Br)c2)nc2c1c(Br)cc1c2ccn1Cc1cccc(Br)c1] | ND |
| 49 | 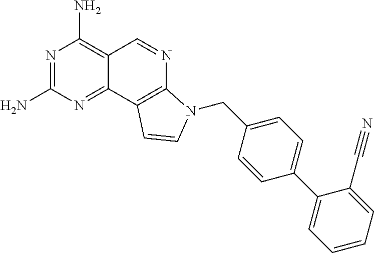 [SMILES: N#Cc1ccccc1-c1ccc(Cn2ccc3c4nc(N)nc(N)c4cnc32)cc1] | ND |
| 50 | 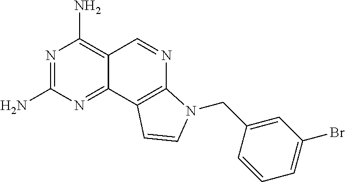 [SMILES: Nc1nc(N)c2cnc3c(ccn3Cc3cccc(Br)c3)c2n1] | ND |
| 51 | 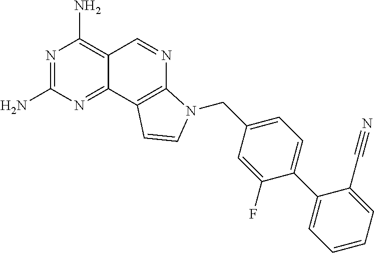 [SMILES: N#Cc1ccccc1-c1ccc(Cn2ccc3c4nc(N)nc(N)c4cnc32)cc1F] | ND |
| 52 | 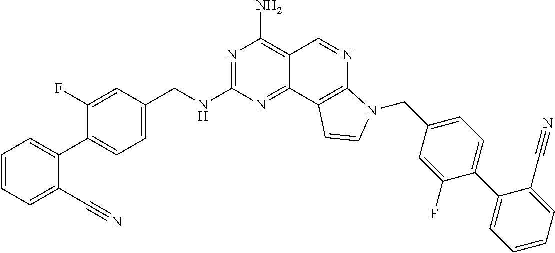 [SMILES: N#Cc1ccccc1-c1ccc(CNc2nc(N)c3cnc4c(ccn4Cc4ccc(-c5ccccc5C#N)c(F)c4)c3n2)cc1F] | ND |
| 53 | 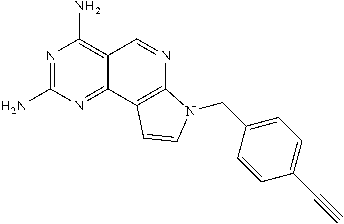 [SMILES: C#Cc1ccc(Cn2ccc3c4nc(N)nc(N)c4cnc32)cc1] | ND |

¹Antibacterial Activity against N. gonorrhoeae ATCC 49226 in TH.

The ability, of a compound disclosed herein to inhibit bacterial growth is shown in Table 2 and can be determined using a method as described in Example 54.

| | Intrinsic MIC ( [symbol] mL) in CAMH¤ | | | | | | | | | | |
|---|---|---|---|---|---|---|---|---|---|---|---|
| | P. | | | | | K. | K. | | | | |
| | aeruginosa | A. [symbol] | E. coli¶ | | | pneumonias¶ | pneumonias¶ | S. [symbol] ¶ | P. mirabilis¶ | MSSA¶ | MRSA¶ |
| | ATCC¶ | ATCC | ATCC | E. coli | E. coli¶ | ATCC¶ | ATCC¶ | ATCC | ATCC | ATCC | ATCC |
| Example #2 | 278538¤ | 19606¤ | 25922¤ | W4573¤ | N43 ¤ | 10031¤ | 13883¤ | 29930¤ | 29906¤ | 19636¤ | 33591¤ |
| 2¤ | >128¤ | 16¤ | 2¤ | 1¤ | ≤0.25¤ | ≤0.25¤ | 4¤ | ≤0.25¤ | 4¤ | ≤0.25¤ | ≤0.25¤ |
| 3¤ | >64¤ | 16¤ | 32¤ | >64¤ | 1¤ | 4¤ | >64¤ | 16¤ | >64¤ | 2¤ | 2¤ |
| 4¤ | >64¤ | 16¤ | 4¤ | 2¤ | <0.125¤ | <0.125¤ | 8¤ | 1¤ | >64¤ | ≤0.125¤ | ≤0.125¤ |
| 6¤ | >64¤ | 32¤ | 4¤ | 4¤ | ≤0.015¤ | ≤0.125¤ | 16¤ | 0.25¤ | >64¤ | 0.5¤ | 0.5¤ |
| 7¤ | >64¤ | 16¤ | 2¤ | 2¤ | ≤0.015¤ | ≤0.125¤ | 8¤ | ≤0.125¤ | >64¤ | ≤0.25¤ | ≤0.25¤ |
| 16¤ | >64¤ | >64¤ | 4¤ | 2¤ | ≤0.015¤ | ≤0.125¤ | >64¤ | 0.5¤ | >64¤ | ≤0.25¤ | ≤0.25¤ |
| 21¤ | >128¤ | 16¤ | >64¤ | >64¤ | 8¤ | 8¤ | >64¤ | >64¤ | >64¤ | 4¤ | 4¤ |
| 26¤ | >128¤ | 32¤ | 16¤ | 16¤ | <0.125¤ | 2¤ | 64¤ | 4¤ | >64¤ | 1¤ | 1¤ |
| 27¤ | >64¤ | 8¤ | 4¤ | 2¤ | <0.125¤ | <0.125¤ | 4¤ | 1¤ | >64¤ | ≤0.125¤ | ≤0.125¤ |
| 31¤ | >128¤ | >64¤ | >64¤ | >64¤ | <0.125¤ | 0.5¤ | >64¤ | 2¤ | >64¤ | 0.25¤ | 0.5¤ |
| 32¤ | ND¤ | ND¤ | ND¤ | ND¤ | ND¤ | ND¤ | ND¤ | ND¤ | ND¤ | ND¤ | ND¤ |
| 33¤ | ND¤ | ND¤ | ND¤ | ND¤ | ND¤ | ND¤ | ND¤ | ND¤ | ND¤ | ND¤ | ND¤ |
| 34¤ | >64¤ | >64¤ | ND¤ | 2¤ | <0.125¤ | <0.125¤ | 64¤ | 4¤ | 64¤ | ≤0.063¤ | ≤0.063¤ |
| 35¤ | >64¤ | 64¤ | ND¤ | 8¤ | <0.125¤ | 0.25¤ | >64¤ | 4¤ | >64¤ | 0.5¤ | 1¤ |
| 36¤ | >64¤ | >64¤ | ND¤ | 8¤ | <0.125¤ | 1¤ | >64¤ | 8¤ | >64¤ | 0.5¤ | 0.5¤ |
| 37¤ | >64¤ | >64¤ | ND¤ | 8¤ | <0.125¤ | 0.25¤ | >64¤ | 4¤ | >64¤ | 0.125¤ | 0.125¤ |
| 38¤ | >64¤ | >64¤ | ND¤ | 1¤ | <0.125¤ | <0.125¤ | 16¤ | 2¤ | >64¤ | ≤0.063¤ | ≤0.063¤ |
| 39¤ | >64¤ | >64¤ | ND¤ | 8¤ | ≤0.063¤ | 1¤ | >64¤ | 1¤ | >64¤ | ≤0.063¤ | ≤0.063¤ |
| 40¤ | ND¤ | ND¤ | ND¤ | ND¤ | ND¤ | ND¤ | ND¤ | 64¤ | >64¤ | 0.25¤ | ≤0.063¤ |
| 41¤ | >64¤ | >64¤ | ND¤ | 2¤ | <0.125¤ | <0.125¤ | 8¤ | 2¤ | >64¤ | 0.5¤ | 1¤ |
| 42¤ | >64¤ | >64¤ | ND¤ | 4¤ | <0.125¤ | <0.125¤ | 32¤ | ≤0.125¤ | 32¤ | ≤0.063¤ | ≤0.063¤ |
| 43¤ | >64¤ | >64¤ | ND¤ | >64¤ | 32¤ | >64¤ | >64¤ | >64¤ | >64¤ | 32¤ | 2¤ |
| 44¤ | >64¤ | >64¤ | ND¤ | >64¤ | 4¤ | 32¤ | >64¤ | 32¤ | >64¤ | 2¤ | 1¤ |
| 45¤ | >64¤ | >64¤ | ND¤ | >64¤ | 32¤ | >64¤ | >64¤ | >64¤ | >64¤ | 2¤ | 2¤ |
| 46¤ | ND¤ | ND¤ | ND¤ | ND¤ | ND¤ | ND¤ | ND¤ | 4¤ | >64¤ | 4¤ | 2¤ |
| 47¤ | ND¤ | ND¤ | ND¤ | ND¤ | ND¤ | ND¤ | ND¤ | 2¤ | 8¤ | 4¤ | 4¤ |
| 48¤ | ND¤ | ND¤ | ND¤ | ND¤ | ND¤ | ND¤ | ND¤ | >64¤ | >64¤ | >64¤ | >64¤ |
| 49¤ | >64¤ | >64¤ | ND¤ | >64¤ | <0.125¤ | 0.5¤ | >64¤ | 1¤ | >64¤ | ≤0.063¤ | ≤0.063¤ |
| 50¤ | >64¤ | >64¤ | ND¤ | 32¤ | ≤0.063¤ | 1¤ | >64¤ | 8¤ | >64¤ | ≤0.063¤ | ≤0.063¤ |
| 51¤ | ND¤ | ND¤ | ND¤ | ND¤ | ND¤ | ND¤ | ND¤ | 8¤ | >64¤ | ≤0.063¤ | ≤0.063¤ |
| 52¤ | ND¤ | ND¤ | ND¤ | ND¤ | ND¤ | ND¤ | ND¤ | >64¤ | >64¤ | >64¤ | >64¤ |
| 53¤ | ND¤ | ND¤ | ND¤ | ND¤ | ND¤ | ND¤ | ND¤ | ≥64¤ | >64¤ | ≤0.063¤ | ≤0.063¤ |
| AZM¤ | 64¤ | 64¤ | 8¤ | 4¤ | 1¤ | 2¤ | 32¤ | 32¤ | 64¤ | 1¤ | 1¤ |
| TMP¤ | >64¤ | 16¤ | 0.5¤ | 0.5¤ | <0.125¤ | 0.25¤ | 2¤ | ≤0.25¤ | 1¤ | 0.5¤ | 0.5¤ |
| VAN¤ | —¤ | —¤ | —¤ | —¤ | —¤ | —¤ | —¤ | —¤ | —¤ | 2¤ | 2¤ |

MSSA methicillin-susceptable Staphalococcus-aureus-
MRSA methicillin-resistant S. aureus-
ND not done-
AZM azithromycin-
TMP [symbol] -
VAN [symbol] ¤ [symbol]
indicates data missing or illegible when filed

The invention will now be illustrated by the following non-limiting examples.

### Preparation of Amine Intermediates A, B, C, D, and E

### Section 1: Synthetic Procedure for the Preparation of Intermediate A, B, C, D and E

| Intermediate | A | B | C |
|---|---|---|---|
| Structure | 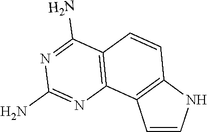 | 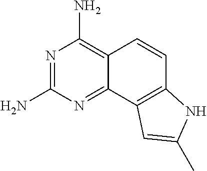 | 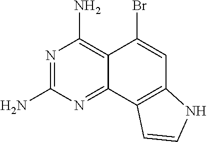 |
| | Intermediate | D | E |
| | Structure | 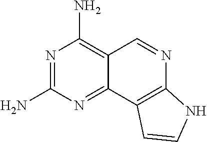 | 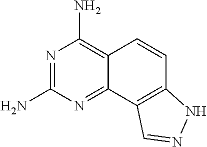 |

### Preparation of Intermediate A: 7H-pyrrolo[2,3-h]quinazoline-2,4-diamine

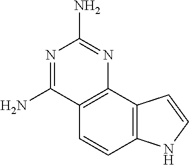 [SMILES: Nc1nc(N)c2ccc3[nH]ccc3c2n1]

### 7H-pyrrolo[2,3-h]quinazoline-2,4-diamine

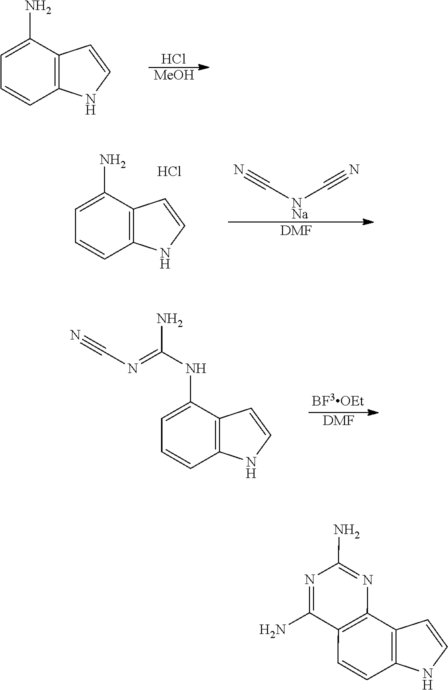

To a solution of 2-cyano-1-(1H-indol-4-yl)guanidine (5.2 g, 26 mmol) in dimethoxyethane (200 mL) was added BF₃·Et₂O (16 mL, 130 mmol) slowly at room temperature. It was stirred at 80° C. overnight under N₂, then the solvent was removed in vacuo and the residue was suspended in methanol (50 mL). To the solution NH₃/H₂O (35 mL) was added. It was stirred at r.t. for 2 hours then concentrated and loaded on silica gel. It was purified by column chromatography on silica gel using MeOH in DCM as eluents to provide a brown powder (3.3 g, 64% yield). ¹HNMR (300 MHz, DMSO-d₆) δ 11.72 (s br, 1H), 8.02 (s br, 1H), 7.73 (d, J=9.0 Hz, 1H), 7.42 (s, 1H), 7.29 (d, J=9.0 Hz, 1H), 6.90 (br s, 1H), 6.85 (s, 1H). MS (ESI): Calcd for C₁₀H₁₀N₅⁺ 200.09 [M+H]⁺, found 199.80 [M+H]⁺.

The requisite intermediate was prepared as follows:

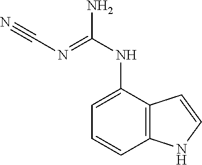 [SMILES: N#C/N=C(\N)Nc1cccc2[nH]ccc12]

### 2-cyano-1-(1H-indol-4-yl)guanidine

To a solution of 4-aminoindole (5.29 g, 40 mmol) in methanol (150 mL) was added HCl solution (4 M in dioxane, 12.5 mL, 50 mmol) at r.t. slowly. The reaction mixture was stirred at r.t. for 10 min then the solvent was removed in vacuo. The residue was dissolved in DMF (60 mL) and sodium dicyanamide (8.90 g, 100 mmol) was added at r.t. The reaction mixture was heated at 45° C. overnight. Then DMF was removed in vacuo. The residue was treated with water and the precipitate was filtered off, washed with water, and dried in vacuo. The crude product was collected as a grey powder (5.3 g, 67% yield) which was used for the next step reaction without purification. ¹H NMR (300 MHz, DMSO-d₆) δ 11.20 (s, 1H), 8.88 (s, 1H), 7.32 (m, 1H), 7.19 (t, J=8.1, Hz, 2H), 7.02 (t, J=7.8 Hz, 1H), 6.94 (s, 2H), 6.44 (s, 1H). MS (ESI): Calcd for C₁₀H₁₀N₅⁺ 200.09 [M+H]⁺, found 199.85 [M+H]⁺.

### Preparation of Intermediate B: 8-methyl-7H-pyrrolo[2,3-h]quinazoline-2,4-diamine

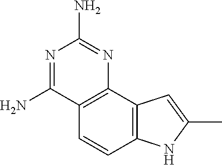 [SMILES: Cc1cc2c(ccc3c(N)nc(N)nc32)[nH]1]

### 8-methyl-7H-pyrrolo[2,3-h]quinazoline-2,4-diamine

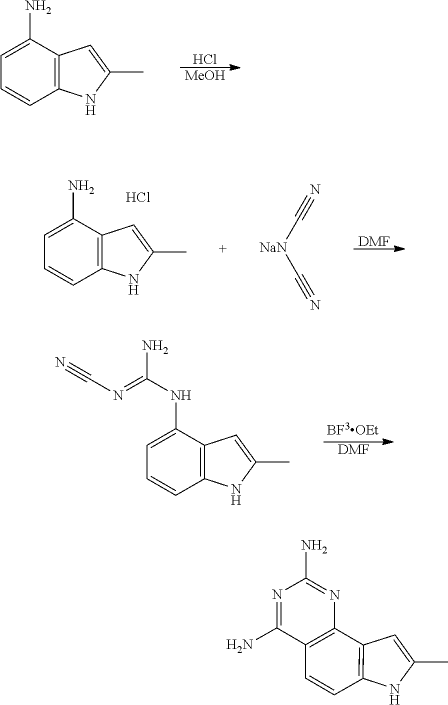

To a solution of 2-cyano-1-(2-methyl-1H-indol-4-yl)guanidine (0.82 g, 3.8 mmol) in dimethoxyethane (30 mL) was added BF₃·Et₂O (2.4 mL, 20 mmol) slowly at room temperature. It was stirred at 80° C. overnight under N₂, then the solvent was removed in vacuo and the residue was suspended in small amount of methanol (10 mL). To the solution NH₃·H₂O (5 mL) was added. It was stirred at r.t. for 2 hours then concentrated and loaded on silica gel. It was purified by column chromatography on silica gel using MeOH in DCM to provide a brown powder (0.68 g, 83% yield). ¹HNMR (300 MHz, DMSO-d₆) δ 11.82 (s br, 1H), 8.67 (s br, 1H), 8.45 (s br, 1H), 7.75 (d, J=9.0 Hz, 1H), 7.31 (d, J=9.0 Hz, 1H), 7.23 (s br, 1H), 7.06 (s br, 1H), 6.89 (s br, 1H), 6.59 (s, 1H), 2.44 (s, 3H). MS (ESI). Calcd for C₁₁H₁₂N₅⁺ 214.10 [M+H]⁺, found 213.85 [M+H]⁺.

The requisite intermediate was prepared as follows:

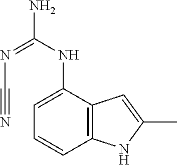 [SMILES: Cc1cc2c(N/C(N)=N\C#N)cccc2[nH]1]

### 2-cyano-1-(2-methyl-1H-indol-4-yl)guanidine

To a solution of 4-aminoindole (0.83 g, 5.7 mmol) in methanol (30 mL) was added HCl solution (4 M in dioxane, 1.7 mL, 6.8 mmol) at r.t. slowly. The reaction mixture was stirred at r.t. for 5 min then the solvent was removed in vacuo. The residue was dissolved in DMF (12 mL) and sodium dicyanamide (1.26 g, 14.2 mmol) was added at r.t. The reaction mixture was heated at 45° C. overnight. Then DMF was removed in vacuo. The residue was treated with water, and the precipitate was filtered off, washed with water, and dried in vacuo. The crude product was collected as a grey powder (0.83 g, 69% yield) which was used for the next step reaction without purification. ¹HNMR (300 MHz, DMSO-d₆) δ 11.02 (s, 1H), 8.79 (s, 1H), 7.09 (t, J=7.5 Hz, 1H), 7.04 (s, 1H), 6.91 (t, J=7.8 Hz, 1H), 6.88 (s, 1H), 6.11 (s, 1H), 2.36 (s, 3H). MS (ESI): Calcd for C₁₁H₁₂N₅⁺ 214.10 [M+H]⁺, found 213.90 [M+H]⁺.

### Preparation of Intermediate C: 5-bromo-7H-pyrrolo[2,3-h]quinazoline-2,4-diamine

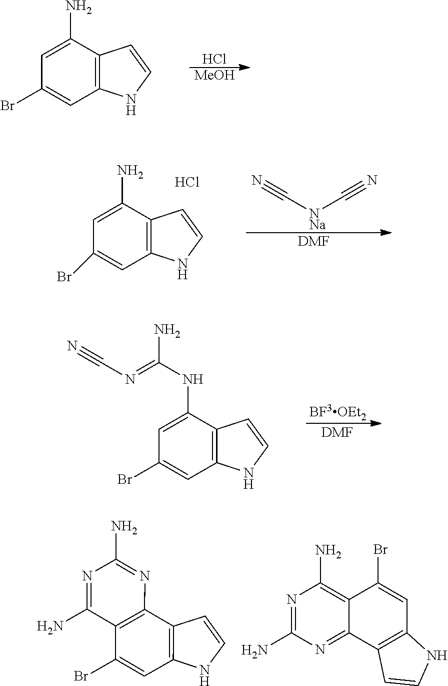

### 5-bromo-7H-pyrrolo[2,3-h]quinazoline-2,4-diamine

To a solution of 6-bromo-1H-indol-4-amine (0.25 g, 1.2 mmol) in methanol (10 mL) was added HCl solution (4 M in dioxane, 0.4 mL, 1.6 mmol) at r.t. The reaction mixture was stirred at r.t. then the solvent was removed in vacuo. The residue was dissolved in DMF (2 mL) and sodium dicyanamide (0.27 g, 3 mmol) was added at r.t. The reaction mixture was heated at 45° C. overnight. Then DMF was removed in vacuo. The residue was treated with water, and the precipitate was filtered off, washed with water, and dried in vacuo. The crude product was collected as a grey powder. It was dissolved in dimethoxyethane (10 mL) then BF₃·Et₂O (0.8 mL, 6 mmol) was added slowly at room temperature. It was stirred at 65° C. for 2 hours under N₂, then the solvent was removed in vacuo and the residue was suspended in small amount of methanol. To the solution NH₃·H₂O (0.5 mL) was added. It was stirred at r.t. then concentrated and loaded on silica gel. It was purified by column chromatography on silica gel using 0-25% MeOH (containing 2% NH₃·H₂O) in DCM to provide a brown powder (0.10 g, 30% yield). ¹HNMR (300 MHz, MeOH-d₄) δ 7.78 (d, J=0.8 Hz, 1H), 7.47 (d, J=3.3 Hz, 1H), 6.93 (dd, J=3.3, 0.8 Hz, 1H). MS (ESI): Calcd for C₁₀H₁₀BrN₆⁺ 278.00 [M+H]⁺. found 277.85 [M+H]⁺.

### Preparation of Intermediate D: 7H-pyrrolo[3′,2′:5,6]pyrido[4,3-d]pyrimidine-2,4-diamine

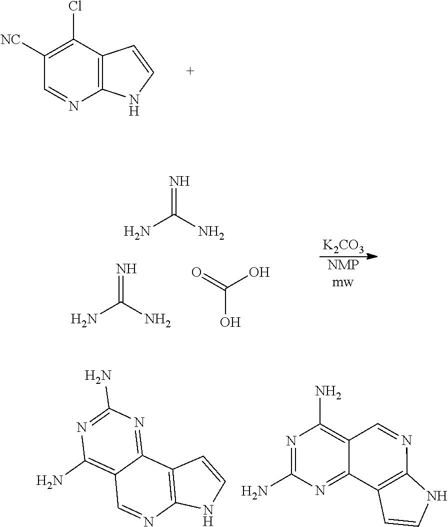

### 7H-pyrrolo[3′,2′:5,6]pyrido[4,3-d]pyrimidine-2,4-diamine

To a mixture of 4-chloro-1H-pyrrolo[2,3-b]pyridine-5-carbonitrile (177 mg, 1 mmol), guanidine carbonate (721 mg, 4 mmol) and anhydrous potassium carbonate (276 mg, 2 mmol) NMP (15 mL) was added. The resulting mixture was sealed under nitrogen and heated via microwave for 3.5 hours at 150° C. After cooling to room temperature, the solvent was removed under vacuo, the residue was loaded on silica gel and purified on column chromatography on silica gel with 0-50% MeOH (containing 2% NH₃·H₂O) in DCM to collect the product as a pale brown powder (136 mg, 68% yield). ¹HNMR (300 MHz, DMSO-d₆) δ 12.11 (s br, 1H), 8.94 (s br, 1H), 7.43 (dd, J=6.2, 1.78 Hz, 1H), 6.96 (s br, 2H), 6.79 (dd, J=6.2, 1.78 Hz, 1H), 6.41 (s br, 2H). MS (ESI): Calcd for C₉H₉N₆⁺ 200.95 [M+H]⁺, found 201.05 [M+H]+.

### Preparation of Intermediate E: 7H-pyrazolo[3,4-h]quinazoline-2,4-diamine

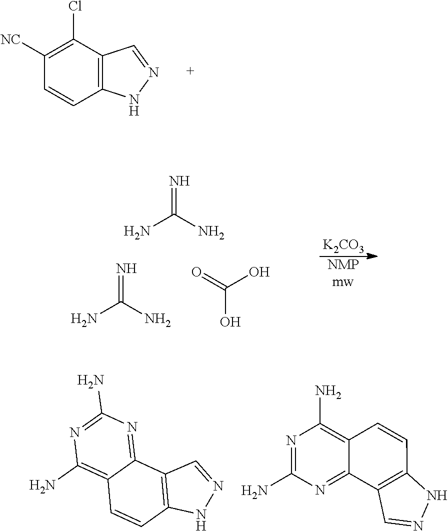

### 7H-pyrazolo[3,4-h]quinazoline-2,4-diamine

To a mixture of 4-chloro-1H-indazole-5-carbonitrile (100 mg, 0.56 mmol), guanidine carbonate (406 mg, 2.24 mmol) and anhydrous potassium carbonate (155 mg, 1.12 mmol) was added NMP (8 mL). The resulting mixture was sealed under nitrogen and heated via microwave for 2 hours at 150° C. After cooling to room temperature, the solvent was removed under vacuo, the residue was loaded on silica gel and purified on silica gel column with 0-30% MeOH (containing 2% NH₃·H₂O) in DCM to collect the product as a pale brown powder (38 mg, 34% yield). ¹HNMR (300 MHz, MeOH-d₄) δ 8.43 (s, 1H), 7.85 (d, J=8.4 Hz, 1H), 7.34 (d, J=8.4 Hz, 1H). MS (ESI): Calcd for C₉H₉N₆⁺ 201.09 [M+H]⁺, found 200.90 [M+H]⁺.

### General Synthetic Procedure for the Preparation of Examples Compounds.

To a solution of intermediates: A-E or 7H-pyrrolo[2,3-h]quinazoline-2,4-diamine (1 eq.) in anhydrous DMF (1 mL/mmol) was added NaH (60% in mineral oil, 1.5 eq.) under nitrogen. After stirring at r.t. for 10 min, the bromide or acid anhydride (1 eq.) was added. The reaction mixture was stirred at r.t. overnight, then treated with water, the precipitate was filtered, washed with water, and dried. Then it was loaded on silica gel and purified by column chromatography on silica gel using MeOH in DCM as eluents.

Example 1 describes a representative synthesis using the general procedure described in the paragraph above.

### Example 1. Preparation of 7-benzyl-7H-pyrrolo[2,3-h]quinazoline-2,4-diamine

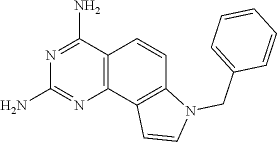 [SMILES: Nc1nc(N)c2ccc3c(ccn3Cc3ccccc3)c2n1]

### 7-benzyl-7H-pyrrolo[2,3-h]quinazoline-2,4-diamine

To a solution of 7H-pyrrolo[2,3-h]quinazoline-2,4-diamine (40 mg, 0.2 mmol) in anhydrous DMF (0.2 mL) was added NaH (60% in mineral oil, 12 mg, 0.3 mmol) under nitrogen. After stirring at r.t. for 10 min, benzyl bromide (24 μL, 0.2 mmol) was added. The reaction mixture was stirred at r.t. overnight, then treated with water, the precipitate was filtered, washed with water, and dried. Then it was suspended in DCM and loaded on silica gel, purified by column chromatography on silica gel using 0-10% MeOH in DCM as eluents to give the product as a white powder (12 mg, 21% yield). ¹H NMR (300 MHz, CD₃COCD₃) δ 7.60 (d, J=8.7 Hz, 1H), 7.31 (m, 3H), 7.26 (m, 2H), 7.16 (m, 2H), 7.06 (d, J=3.0 Hz, 1H), 6.50 (s br, 2H), 5.52 (s br, 2H), 5.46 (s, 2H). MS (ESI): Calcd for C₁₇H₁₆N₅⁺290.13 [M+H]⁺, found 289.80 [M+H]⁺.

Examples 2-53 were prepared following the procedure outline for example 1, the general synthetic schemes and in the general synthetic procedure.

### Example 2. Preparation of 7-(4-bromobenzyl)-7H-pyrrolo[2,3-h]quinazoline-2,4-diamine

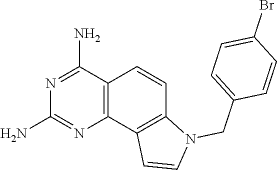 [SMILES: Nc1nc(N)c2ccc3c(ccn3Cc3ccc(Br)cc3)c2n1]

### 7-(4-bromobenzyl)-7H-pyrrolo[2,3-h]quinazoline-2,4-diamine

Pale brown powder (182 mg, 20% yield) was obtained from 7H-pyrrolo[2,3-h]quinazoline-2,4-diamine (500 mg, 2.5 mmol). ¹HNMR (300 MHz, DMSO-d₆) δ 7.63 (d, J=9.3 Hz, 1H), 7.49 (d, J=8.4 Hz, 2H), 7.41 (m, 1H), 7.19 (m, 1H), 7.11 (d, J=8.4 Hz, 2H), 6.80 (d, J=3.0 Hz, 1H), 6.08 (br, 2H), 5.43 (s, 2H). MS (ESI): Calcd for C₁₇H₁₅B_{rN5}⁺ 368.04 [M+H]⁺, found 367.85 [M+H]⁺.

### Example 3. Preparation of 7-(3-bromobenzyl)-7H-pyrrolo[2,3-h]quinazoline-2,4-diamine

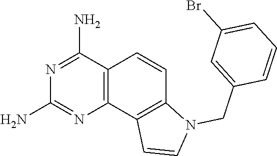 [SMILES: Nc1nc(N)c2ccc3c(ccn3Cc3cccc(Br)c3)c2n1]

### 7-(3-bromobenzyl)-7H-pyrrolo[2,3-h]quinazoline-2,4-diamine

White powder (19 mg, 26% yield) was obtained from 7H-pyrrolo[2,3-h]quinazoline-2,4-diamine (40 mg, 0.2 mmol). MS (ESI): Calcd for C₁₇H₁₅BrN₅⁺ 368.04 [M+H]⁺, found 367.85 [M+H]⁺.

### Example 4. Preparation of 7-(2-bromobenzyl)-7H-pyrrolo[2,3-h]quinazoline-2,4-diamine

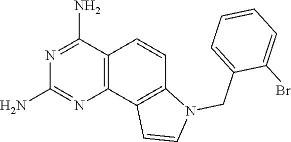 [SMILES: Nc1nc(N)c2ccc3c(ccn3Cc3ccccc3Br)c2n1]

### 7-(2-bromobenzyl)-7H-pyrrolo[2,3-h]quinazoline-2,4-diamine

White powder (33 mg, 18% yield) was obtained from 7H-pyrrolo[2,3-h]quinazoline-2,4-diamine (100 mg, 0.5 mmol). ¹H NMR (300 MHz, CDCl₃) δ 7.61 (m, 1H), 7.19 (m, 1H), 7.15 (m, 2H), 7.13 (m, 2H), 7.06 (d, J=8.7 Hz, 1H), 6.45 (m, 1H), 5.44 (s, 2H), 5.29 (s br, 2H), 4.91 (s br, 2H). MS (ESI): Calcd for C₁₇H₁₅BrN₅⁺ 368.04[M+H]⁺, found 367.90 [M+H]⁺.

### Example 5. Preparation of 4-((2,4-diamino-7H-pyrrolo[2,3-h]quinazolin-7-yl)methyl)benzonitrile

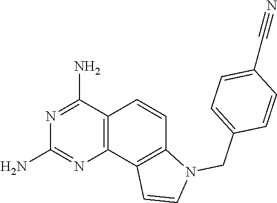 [SMILES: N#Cc1ccc(Cn2ccc3c4nc(N)nc(N)c4ccc32)cc1]

### 4-((2,4-diamino-7H-pyrrolo[2,3-h]quinazolin-7-yl)methyl)benzonitrile

White powder (38 mg, 24% yield) was obtained from 7H-pyrrolo[2,3-h]quinazoline-2,4-diamine (100 mg, 0.5 mmol). ¹H NMR (300 MHz, CDCl₃) δ 7.59 (d, J=8.4 Hz, 2H), 7.28 (d, J=9.0 Hz, 1H), 7.19 (d, J=9.0 Hz, 1H), 7.14 (d, J=9.0 Hz, 1H), 7.12 (d, J=8.7 Hz, 2H), 6.99 (d, J=8.7 Hz, 1H), 5.46 (s, 2H), 5.28 (s br, 2H), 4.90 (s br, 2H). MS (ESI): Calcd for C₁₈H₁₅N₆⁺ 315.13 [M+H]⁺, found 314.90 [M+H]⁺.

### Example 6. Preparation of 3-((2,4-diamino-7H-pyrrolo[2,3-h]quinazolin-7-yl)methyl)benzonitrile

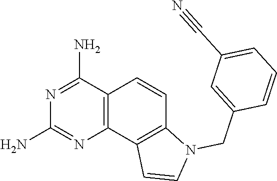 [SMILES: N#Cc1cccc(Cn2ccc3c4nc(N)nc(N)c4ccc32)c1]

### 3-((2,4-diamino-7H-pyrrolo[2,3-h]quinazolin-7-yl)methyl)benzonitrile

White powder (31 mg, 20% yield) was obtained from 7H-pyrrolo[2,3-h]quinazoline-2,4-diamine (100 mg, 0.5 mmol). MS (ESI): Calcd for C₁₈H₁₅N₆⁺315.13 [M+H]⁺, found 314.90 [M+H]⁺.

### Example 7. Preparation of 7-(4-(trifluoromethyl)benzyl)-7H-pyrrolo[2,3-h]quinazoline-2,4-diamine

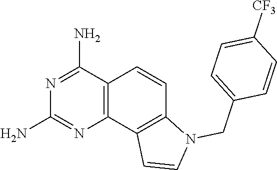 [SMILES: Nc1nc(N)c2ccc3c(ccn3Cc3ccc(C(F)(F)F)cc3)c2n1]

### 7-(4-(trifluoromethyl)benzyl)-7H-pyrrolo[2,3-h]quinazoline-2,4-diamine

White powder (34 mg, 19% yield) was obtained from 7H-pyrrolo[2,3-h]quinazoline-2,4-diamine (100 mg, 0.5 mmol). ¹H NMR (300 MHz, CDCl₃) δ 7.54 (d, J=7.2 Hz, 2H), 7.28 (m, 1H), 7.17 (m, 4H), 7.03 (d, J=9.0 Hz, 1H), 5.45 (s, 2H), 5.28 (s br, 2H), 4.89 (s br, 2H). MS (ESI): Calcd for C₁₈H₁₅F₃N₅⁺ 358.12 [M+H]⁺, found 357.90 [M+H]⁺.

### Example 8. Preparation of 7-(3-(trifluoromethyl)benzyl)-7H-pyrrolo[2,3-h]quinazoline-2,4-diamine

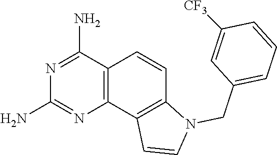 [SMILES: Nc1nc(N)c2ccc3c(ccn3Cc3cccc(C(F)(F)F)c3)c2n1]

### 7-(3-(trifluoromethyl)benzyl)-7H-pyrrolo[2,3-h]quinazoline-2,4-diamine

White powder (78 mg, 44% yield) was obtained from 7H-pyrrolo[2,3-h]quinazoline-2,4-diamine (100 mg, 0.5 mmol). ¹H NMR (300 MHz, CDCl₃) δ 7.53 (d, J=7.8 Hz, 1H), 7.42 (s, 1H), 7.38 (d, J=7.5 Hz, 1H), 7.29 (d, J=9.0 Hz, 1H), 7.17 (m, 3H), 7.05 (d, J=8.7 Hz, 1H), 5.44 (s, 2H), 5.29 (s br, 2H), 4.91 (s br, 2H). MS (ESI): Calcd for C₁₅H₁₅F₃N₅⁺ 358.12 [M+H]⁺, found 357.90 [M+H]⁺.

### Example 9. Preparation of 7-(4-(trifluoromethoxy)benzyl)-7H-pyrrolo[2,3-h]quinazoline-2,4-diamine

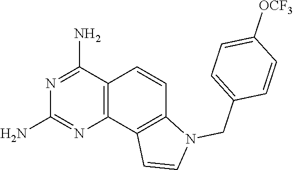 [SMILES: Nc1nc(N)c2ccc3c(ccn3Cc3ccc(OC(F)(F)F)cc3)c2n1]

### 7-(4-(trifluoromethoxy)benzyl)-7H-pyrrolo[2,3-h]quinazoline-2,4-diamine

White powder (36 mg, 19% yield) was obtained from 7H-pyrrolo[2,3-h]quinazoline-2,4-diamine (100 mg, 0.5 mmol). ¹H NMR (300 MHz, CDCl₃) δ 7.56 (d, J=7.2 Hz, 1H), 7.38 (m, 2H), 7.29 (d, J=9.0 Hz, 1H), 7.23 (m, 1H), 7.15 (dd, J=13.8, 3.0 Hz, 2H), 7.02 (d, J=8.7 Hz, 1H), 5.42 (s, 2H), 5.30 (s br, 2H), 4.92 (s br, 2H). MS (ESI): Calcd for C₁₅H₁₅F₃N₅O⁺ 374.12 [M+H]⁺, found 373.90 [M+H]⁺.

### Example 10. Preparation of 7-(4-isopropylbenzyl)-7H-pyrrolo[2,3-h]quinazoline-2,4-diamine

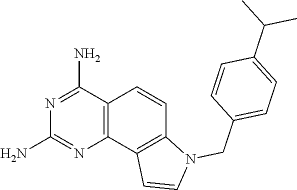 [SMILES: CC(C)c1ccc(Cn2ccc3c4nc(N)nc(N)c4ccc32)cc1]

### 7-(4-isopropylbenzyl)-7H-pyrrolo[2,3-h]quinazoline-2,4-diamine

White powder (92 mg, 55% yield) was obtained from 7H-pyrrolo[2,3-h]quinazoline-2,4-diamine (100 mg, 0.5 mmol). ¹H NMR (300 MHz, CDCl₃) δ 7.27 (d, J=9.0 Hz, 1H), 7.19 (m, 5H), 7.14 (m, 2H), 7.01 (d, J=8.1 Hz, 2H), 5.35 (s, 2H), 5.28 (s br, 2H), 4.89 (s br, 2H), 2.86 (m, 1H), 1.21 (d, J=6.9 Hz, 6H). MS (ESI): Calcd for C₂₀H₂₂N₅⁺ 332.18 [M+H]⁺, found 332.00 [M+H]⁺.

### Example 11. Preparation of 7-(3-fluoro-4-(trifluoromethyl)benzyl)-7H-pyrrolo[2,3-h]quinazoline-2,4-diamine

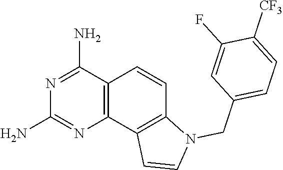 [SMILES: Nc1nc(N)c2ccc3c(ccn3Cc3ccc(C(F)(F)F)c(F)c3)c2n1]

### 7-(3-fluoro-4-(trifluoromethyl)benzyl)-7H-pyrrolo[2,3-h]quinazoline-2,4-diamine

White powder (59 mg, 31% yield) was obtained from 7H-pyrrolo[2,3-h]quinazoline-2,4-diamine (100 mg, 0.5 mmol). ¹H NMR (300 MHz, CDCl₃) δ 7.53 (t, J=7.8 Hz, 1H), 7.30 (d, J=8.7 Hz, 2H), 7.16 (d, J=3.0 Hz, 1H), 7.02 (d, J=8.7 Hz, 1H), 6.90 (d, J=9.0 Hz, 1H), 6.84 (d, J=11.1 Hz, 1H), 5.44 (s, 2H), 5.35 (s br, 2H). MS (ESI): Calcd for C₁₈H₁₄F₄N₅⁺ 376.11 [M+H]⁺, found 375.95 [M+H]⁺.

### Example 12. Preparation of 7-(5-chloro-2-fluorobenzyl)-7H-pyrrolo[2,3-h]quinazoline-2,4-diamine

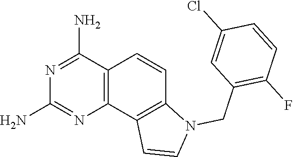 [SMILES: Nc1nc(N)c2ccc3c(ccn3Cc3cc(Cl)ccc3F)c2n1]

### 7-(5-chloro-2-fluorobenzyl)-7H-pyrrolo[2,3-h]quinazoline-2,4-diamine

White powder (62 mg, 36% yield) was obtained from 7H-pyrrolo[2,3-h]quinazoline-2,4-diamine (100 mg, 0.5 mmol). ¹H NMR (300 MHz, CDCl₃) δ 7.32 (d, J=9.0 Hz, 1H), 7.17 (m, 3H), 7.12 (d, J=9.0 Hz, 1H), 7.05 (t, J=9.0 Hz, 1H), 6.73 (dd, J=6.9, 3.0 Hz, 1H), 5.39 (s, 2H), 5.29 (s br, 2H), 4.89 (s br, 2H). MS (ESI): Calcd for C₁₇H₁₄ClFN₅⁺ 342.08 [M+H]⁺, found 341.85 [M+H]⁺.

### Example 13. Preparation of 7-(2,5-dichlorobenzyl)-7H-pyrrolo[2,3-h]quinazoline-2,4-diamine

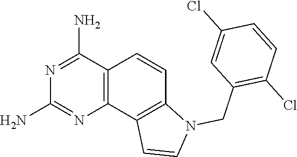 [SMILES: Nc1nc(N)c2ccc3c(ccn3Cc3cc(Cl)ccc3Cl)c2n1]

### 7-(2,5-dichlorobenzyl)-7H-pyrrolo[2,3-h]quinazoline-2,4-diamine

Off-white powder (8 mg, 13% yield) was obtained from 7H-pyrrolo[2,3-h]quinazoline-2,4-diamine (40 mg, 0.2 mmol). MS (ESI): Calcd for C₁₇H₁₄Cl₂N₅⁺ 358.05 [M+H]⁺, found 357.90 [M+H]⁺.

### Example 14. Preparation of 7-(3,5-dimethoxybenzyl)-7H-pyrrolo[2,3-h]quinazoline-2,4-diamine

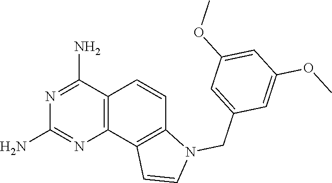 [SMILES: COc1cc(Cn2ccc3c4nc(N)nc(N)c4ccc32)cc(OC)c1]

### 7-(3,5-dimethoxybenzyl)-7H-pyrrolo[2,3-h]quinazoline-2,4-diamine

White powder (108 mg, 62% yield) was obtained from 7H-pyrrolo[2,3-h]quinazoline-2,4-diamine (100 mg, 0.5 mmol). ¹H NMR (300 MHz, CDCl₃) δ 7.27 (d, J=9.0 Hz, 1H), 7.15 (m, 2H), 7.11 (d, J=8.7 Hz, 1H), 6.34 (t, J=2.1 Hz, 1H), 6.21 (d, J=2.4 Hz, 2H), 5.31 (s, 2H), 5.28 (s br, 2H), 4.89 (s br, 2H), 3.68 (s, 6H). MS (ESI): Calcd for C₁₉H₂₀N₅O₂⁺ 350.15 [M+H]⁺, found 349.95 [M+H]⁺.

### Example 15. Preparation of 7-(4-phenoxybenzyl)-7H-pyrrolo[2,3-h]quinazoline-2,4-diamine

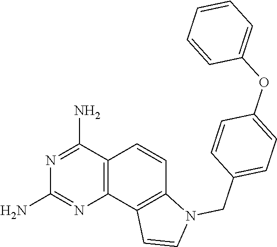 [SMILES: Nc1nc(N)c2ccc3c(ccn3Cc3ccc(Oc4ccccc4)cc3)c2n1]

### 7-(4-phenoxybenzyl)-7H-pyrrolo[2,3-h]quinazoline-2,4-diamine

White powder (35 mg, 33% yield) was obtained from 7H-pyrrolo[2,3-h]quinazoline-2,4-diamine (56 mg, 0.28 mmol). ¹H NMR (300 MHz, CD₃COCD₃) δ 8.19 (br, 2H), 7.92 (d, J=9.0 Hz, 1H), 7.66 (m, 2H), 7.36 (m, 2H), 7.30 (d, J=8.7 Hz, 2H), 7.11 (m, 2H), 6.92-6.99 (m, 4H), 5.59 (s, 2H). MS (ESI): Calcd for C₂₃H₂₀N₅O⁺ 382.16 [M+H]⁺, found 381.95 [M+H]⁺.

### Example 16. Preparation of (4-((2,4-diamino-7H-pyrrolol[2,3-h]quinazolin-7-yl)methyl)phenyl)(phenyl)methanone

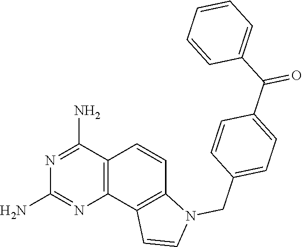 [SMILES: Nc1nc(N)c2ccc3c(ccn3Cc3ccc(C(=O)c4ccccc4)cc3)c2n1]

### (4-((2,4-diamino-7H-pyrrolo[2,3-h]quinazolin-7-yl)methyl)phenyl)(phenyl)methanone

White powder (35 mg, 18% yield) was obtained from 7H-pyrrolo[2,3-h]quinazoline-2,4-diamine (100 mg, 0.5 mmol). ¹H NMR (300 MHz, CDCl₃) δ 7.71-7.80 (m, 5H), 7.57 (m, 2H), 7.45 (m, 3H), 7.17 (m, 2H), 7.08 (d, J=9.0 Hz, 1H), 5.48 (s, 2H), 5.28 (s br, 2H), 4.90 (s br, 2H). MS (ESI): Calcd for C₂₄H₂₀N₅O⁺ 394.16 [M+H]⁺, found 393.95 [M+H]⁺.

### Example 17. Preparation of 7-([1,1′-biphenyl]-4-ylmethyl)-7H-pyrrolo[2,3-h]quinazoline-2,4-diamine

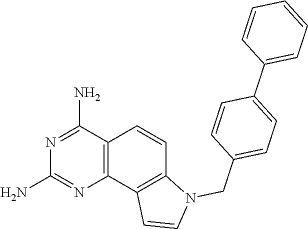 [SMILES: Nc1nc(N)c2ccc3c(ccn3Cc3ccc(-c4ccccc4)cc3)c2n1]

### 7-([1,1′-biphenyl]-4-ylmethyl)-7H-pyrrolo[2,3-h]quinazoline-2,4-diamine

Off-white powder (28 mg, 26% yield) was obtained from 7H-pyrrolo[2,3-h]quinazoline-2,4-diamine (60 mg, 0.3 mmol). ¹H NMR (300 MHz, CD₃COCD₃) δ 7.50-7.58 (m, 4H), 7.36-7.43 (m, 3H), 7.26 (m, 2H), 7.20 (d, J=8.4 Hz, 2H), 7.15 (d, J=9.0 Hz, 1H), 7.06 (d, J=3.0 Hz, 1H), 6.35 (s br, 2H), 5.48 (s, 2H), 5.35 (s br, 2H). MS (ESI): Calcd for C₂₃H₂₀N₅⁺ 366.16 [M+H]⁺, found 366.00 [M+H]⁺.

### Example 18. Preparation of 7-([1,1′-biphenyl]-3-ylmethyl)-7H-pyrrolo[2,3-h]quinazoline-2,4-diamine

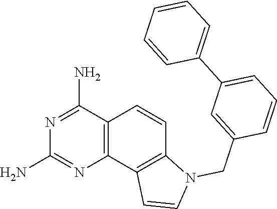 [SMILES: Nc1nc(N)c2ccc3c(ccn3Cc3cccc(-c4ccccc4)c3)c2n1]

### 7-([1,1′-biphenyl]-3-ylmethyl)-7H-pyrrolo[2,3-h]quinazoline-2,4-diamine

Off-white powder (25 mg, 23% yield) was obtained from 7H-pyrrolo[2,3-h]quinazoline-2,4-diamine (60 mg, 0.3 mmol). MS (ESI): Calcd for C₂₃H₂₀N₅⁺ 366.16 [M+H]⁺, found 365.95 [M+H]⁺.

### Example 19. Preparation of 4′-((2,4-diamino-7H-pyrrolo[2,3-h]quinazolin-7-yl)methyl)-[1,1′-biphenyl]-2-carbonitrile

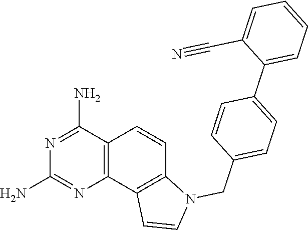 [SMILES: N#Cc1ccccc1-c1ccc(Cn2ccc3c4nc(N)nc(N)c4ccc32)cc1]

### 4′-((2,4-diamino-7H-pyrrolo[2,3-h]quinazolin-7-yl)methyl)-[1,1′-biphenyl]-2-carbonitrile

Off-white powder (27 mg, 23% yield) was obtained from 7H-pyrrolo[2,3-h]quinazoline-2,4-diamine (60 mg, 0.3 mmol). ¹H NMR (300 MHz, CD₃OD) δ 7.79 (d, J=7.8 Hz, 1H), 7.68 (m, 1H), 7.60 (d, J=9.0 Hz, 1H), 7.41-7.56 (m, 5H), 7.36 (d, J=3.0 Hz, 1H), 7.25 (m, 2H), 7.07 (d, J=3.3 Hz, 1H), 5.53 (s, 2H). MS (ESI): Calcd for C₂₄H₁₉N₆⁺ 391.16 [M+H]⁺, found 390.95 [M+H]⁺.

### Example 20. Preparation of methyl 4′-((2,4-diamino-7H-pyrrolo[2,3-h]quinazolin-7-yl)methyl)-[1,1′-biphenyl]-4-carboxylate

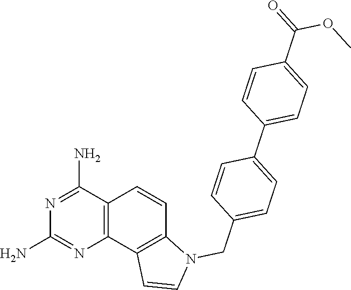 [SMILES: COC(=O)c1ccc(-c2ccc(Cn3ccc4c5nc(N)nc(N)c5ccc43)cc2)cc1]

### methyl 4′-((2,4-diamino-7H-pyrrolo[2,3-h]quinazolin-7-yl)methyl)-[1,1′-biphenyl]-4-carboxylate

Off-white powder (29 mg, 14% yield) was obtained from 7H-pyrrolo[2,3-h]quinazoline-2,4-diamine (100 mg, 0.5 mmol). ¹H NMR (300 MHz, CDCl₃) δ 8.08 (d, J=8.4 Hz, 2H), 7.60 (d, J=8.4 Hz, 2H), 7.55 (d, J=8.1 Hz, 2H), 7.29 (d, J=8.7 Hz, 1H), 7.18 (m, 4H), 7.12 (d, J=8.7 Hz, 1H), 5.45 (s, 2H), 5.27 (s br, 2H), 4.90 (s br, 2H), 3.93 (s, 3H). MS (ESI): Calcd for C₂₅H₂₂N₅O₂⁺ 424.18 [M+H]⁺, found 423.95 [M+H]⁺.

### Example 21. Preparation of 7-(naphthalen-2-ylmethyl)-7H-pyrrolo[2,3-h]quinazoline-2,4-diamine

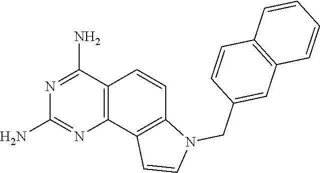 [SMILES: Nc1nc(N)c2ccc3c(ccn3Cc3ccc4ccccc4c3)c2n1]

### 7-(naphthalen-2-ylmethyl)-7H-pyrrolo[2,3-h]quinazoline-2,4-diamine

White powder (20 mg, 18% yield) was obtained from 7H-pyrrolo[2,3-h]quinazoline-2,4-diamine (64 mg, 0.32 mmol). ¹H NMR (3(0) MHz, CDCl₃) δ 8.17 (s, 1H), 7.92 (m, 3H), 7.80 (m, 1H), 7.73 (m, 1H), 7.59 (m, 2H), 7.44 (m, 3H), 5.33 (s, 2H). MS (ESI): Calcd for C₂₁H₁₈N₅⁺ 340.15 [M+H]⁺, found 339.90 [M+H]⁺.

### Example 22. Preparation of 7-((5-bromopyridin-2-yl)methyl)-7H-pyrrolo[2,3-h]quinazoline-2,4-diamine

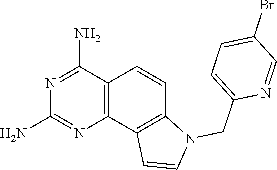 [SMILES: Nc1nc(N)c2ccc3c(ccn3Cc3ccc(Br)cn3)c2n1]

### 7-((5-bromopyridin-2-yl)methyl)-7H-pyrrolo[2,3-h]quinazoline-2,4-diamine

Pale brown powder (27 mg, 15% yield) was obtained from 7H-pyrrolo[2,3-h]quinazoline-2,4-diamine (100 mg, 0.5 mmol). ¹H NMR (300 MHz, CDCl₃) δ 8.65 (d, J=2.4 Hz, 1H), 7.64 (dd, J=8.4, 2.4 Hz, 1H), 7.28 (d, J=8.7 Hz, 1H), 7.19 (s, 2H), 7.06 (d, J=9.0 Hz, 1H), 6.51 (d, J=8.1 Hz, 1H), 5.47 (s, 2H), 5.27 (s br, 2H), 4.88 (s br, 2H). MS (ESI): Calcd for C₁₆H₁₄BrN₆⁺ 369.05 [M+H]⁺, found 368.85 [M+H]⁺.

### Example 23. Preparation of 7-((1-methyl-1H-benzo[d] midazole-2-yl)methyl)-7H-pyrrolo[2,3-h]quinazoline-2,4-diamine

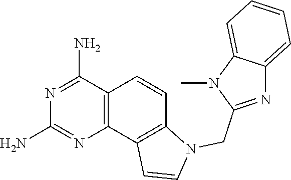 [SMILES: Cn1c(Cn2ccc3c4nc(N)nc(N)c4ccc32)nc2ccccc21]

### 7-((1-methyl-1H-benzo[d] midazole-2-yl)methyl)-7H-pyrrolo[2,3-h]quinazoline-2,4-diamine

Pale brown powder (45 mg, 26% yield) was obtained from 7H-pyrrolo[2,3-h]quinazoline-2,4-diamine (100 mg, 0.5 mmol). ¹H NMR (300 MHz, DMSO-d₆) δ 7.63 (d, J=9.0 Hz, 1H), 7.56 (d, J=7.5 Hz, 1H), 7.50 (d, J=8.1 Hz, 1H), 7.39 (d, J=3.0 Hz, 1H), 7.28 (m, 1H), 7.20 (m, 1H), 7.18 (m, 1H), 6.81 (d, J=3.0 Hz, 1H), 6.00 (br, 2H), 5.79 (s, 2H), 3.73 (s, 3H). MS (ESI): Calcd for C₁₉H₁₈N₇⁺ 343.90 [M+H]⁺, found 344.10 [M+H]⁺.

### Example 24. Preparation of 7-((4-phenylthiazol-2-yl)methyl)-7H-pyrrolo[2,3-h]quinazoline-2,4-diamine

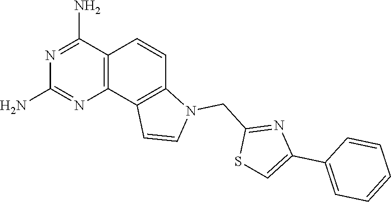 [SMILES: Nc1nc(N)c2ccc3c(ccn3Cc3nc(-c4ccccc4)cs3)c2n1]

### 7-((4-phenylthiazol-2-yl)methyl)-7H-pyrrolo[2,3-h]quinazoline-2,4-diamine

White powder (52 mg, 30% yield) was obtained from 7H-pyrrolo[2,3-h]quinazoline-2,4-diamine (100 mg, 0.5 mmol). ¹H NMR (300 MHz, DMSO-d₆) δ 7.88 (d, J=7.5 Hz, 2H), 7.42 (m, 2H), 7.36 (m, 4H), 7.20 (m, 2H), 5.72 (s, 2H), 5.31 (s br, 2H), 4.90 (s br, 2H). MS (ESI): Calcd for C₂₀H₁₇N₆S⁺ 373.12 [M+H]⁺, found 372.90 [M+H]⁺.

### Example 25. Preparation of 7-(3-methylbut-2-en-1-yl)-7H-pyrrolo[2,3-h]quinazoline-2,4-diamine

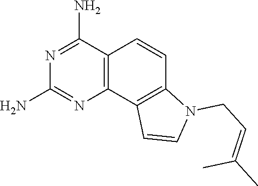 [SMILES: CC(C)=CCn1ccc2c3nc(N)nc(N)c3ccc21]

### 7-(3-methylbut-2-en-1-yl)-7H-pyrrolo[2,3-h]quinazoline-2,4-diamine

White powder (15 mg, 28% yield) was obtained from 7H-pyrrolo[2,3-h]quinazoline-2,4-diamine (40 mg, 0.2 mmol). ¹H NMR (300 MHz, CD₃OD) δ 7.61 (d, J=9.0 Hz, 1H), 7.24 (d, J=9.0 Hz, 1H), 7.20 (d, J=3.0 Hz, 1H), 6.96 (d, J=3.3 Hz, 1H), 5.38 (m, 1H), 4.80 (d, J=6.9 Hz, 2H), 1.86 (s, 3H), 1.77 (s, 3H). MS (ESI): Calcd for C₁₅H₁₈N₅⁺ 268.15 [M+H]⁺, found 267.85 [M+H]⁺.

### Example 26. Preparation of 7-(cyclohex-2-en-1-yl)-7H-pyrrolo[2,3-h]quinazoline-2,4-diamine

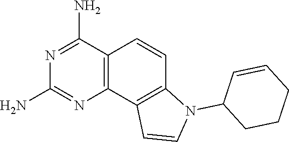 [SMILES: Nc1nc(N)c2ccc3c(ccn3C3C=CCCC3)c2n1]

### 7-(cyclohex-2-en-1-yl)-7H-pyrrolo[2,3-h]quinazoline-2,4-diamine

White powder (8 mg, 12% yield) was obtained from 7H-pyrrolo[2,3-h]quinazoline-2,4-diamine (48 mg, 0.24 mmol). ¹H NMR (300 MHz, CD₃COCD₃) δ 7.93 (d, J=8.7 Hz, 1H), 7.71 (d, J=9.0 Hz, 1H), 7.51 (d, J=3.0 Hz, 1H), 7.04 (d, J=3.0 Hz, 1H), 6.20 (m, 1H), 5.83 (m, 1H), 5.34 (m, 1H), 2.17 (m, 4H), 1.76 (m, 2H).

### Example 27. Preparation of 7-(cyclohexylmethyl)-7H-pyrrolo[2,3-h]quinazoline-2,4-diamine

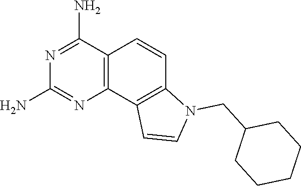 [SMILES: Nc1nc(N)c2ccc3c(ccn3CC3CCCCC3)c2n1]

### 7-(cyclohexylmethyl)-7H-pyrrolo[2,3-h]quinazoline-2,4-diamine

White powder (56 mg, 38% yield) was obtained from 7H-pyrrolo[2,3-h]quinazoline-2,4-diamine (100 mg, 0.5 mmol). ¹H NMR (300 MHz, CDCl₃) δ 7.29 (d, J=9.0 Hz, 1H), 7.17 (d, J=9.0 Hz, 1H), 7.07 (s, 2H), 5.28 (s br, 2H), 4.88 (s br, 2H), 3.98 (d, J=8.4 Hz, 2H), 1.84 (m, 1H), 1.53-1.72 (m, 10H). MS (ESI): Calcd for C₁₇H₂₂N₅⁺ 296.18 [M+H]⁺, found 295.95 [M+H]⁺.

### Example 28. Preparation of 7-(cyclopropylmethyl)-7H-pyrrolo[2,3-h]quinazoline-2,4-diamine

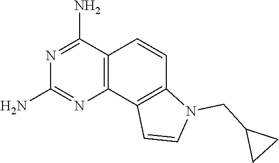 [SMILES: Nc1nc(N)c2ccc3c(ccn3CC3CC3)c2n1]

### 7-(cyclopropylmethyl)-7H-pyrrolo[2,3-h]quinazoline-2,4-diamine

White powder (47 mg, 37% yield) was obtained from 7H-pyrrolo[2,3-h]quinazoline-2,4-diamine (100 mg, 0.5 mmol). ¹H NMR (300 MHz, CDCl₃) δ 7.31 (d, J=9.0 Hz, 1H), 7.23 (d, J=3.0 Hz, 1H), 7.21 (d, J=9.0, Hz, 1H), 7.0) (d, J=3.0 Hz, 1H), 5.27 (s br, 2H), 4.86 (s br, 2H), 4.04 (d, J=6.3 Hz, 2H), 1.29 (m, 1H), 0.65 (m, 2H), 0.38 (m, 2H). MS (ESI): Calcd for C₁₄H₁₆N₅⁺ 254.14 [M+H]⁺, found 253.90 [M+H]⁺.

### Example 29. Preparation of (2,4-diamino-7H-pyrrolo[2,3-h]quinazolin-7-yl)(phenyl)methanone

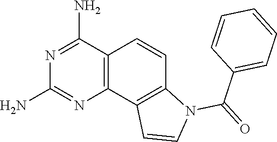 [SMILES: Nc1nc(N)c2ccc3c(ccn3C(=O)c3ccccc3)c2n1]

### (2,4-diamino-7H-pyrrolo[2,3-h]quinazolin-7-yl)(phenyl)methanone

White powder (58 mg, 38% yield) was obtained from 7H-pyrrolo[2,3-h]quinazoline-2,4-diamine (100 mg, 0.5 mmol) and benzoic anhydride (113 mg, 0.5 mmol). ¹H NMR (300 MHz, CD₃COCD₃) δ 7.81 (d, J=8.7 Hz, 1H), 7.64 (m, 2H), 7.53 (m, 1H), 7.49 (m, 1H), 7.42 (m, 2H), 7.16 (d, J=3.9 Hz, 1H), 7.07 (d, J=3.6 Hz, 1H), 5.91 (s br, 2H), 5.02 (s br, 2H). MS (ESI): Calcd for C₁₇H₁₄N₅O⁺ 304.11 [M+H]⁺, found 303.90 [M+H]⁺.

### Example 30. Preparation of 7-(3-bromobenzyl)-8-methyl-7H-pyrrolo[2,3-h]quinazoline-2,4-diamine

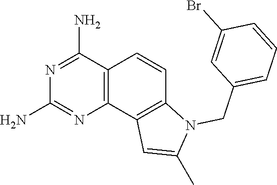 [SMILES: Cc1cc2c3nc(N)nc(N)c3ccc2n1Cc1cccc(Br)c1]

### 7-(3-bromobenzyl)-8-methyl-7H-pyrrolo[2,3-h]quinazoline-2,4-diamine

White powder (40 mg, 21% yield) was obtained from 8-methyl-7H-pyrrolo[2,3-h]quinazoline-2,4-diamine (100 mg, 0.5 mmol). ¹H NMR (300 MHz, DMSO-d₆) δ 7.44 (m, 2H), 7.37 (m, 1H), 7.25 (t, J=7.5 Hz, 1H), 7.01 (s, 1H), 6.91 (d, J=8.1 Hz, 1H), 6.74 (s, 1H), 5.52 (s, 2H), 2.36 (s, 3H). MS (ESI): Calcd for C₂₃H₂₀N₅O⁺ 382.16 [M+H]⁺, found 381.85 [M+H]⁺.

### Example 31. Preparation of 4′-((2,4-diamino-8-methyl-7H-pyrrolo[2,3-h]quinazolin-7-yl)methyl)-[1,1′-biphenyl]-2-carbonitrile

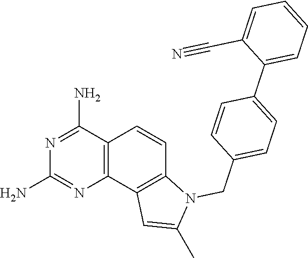 [SMILES: Cc1cc2c3nc(N)nc(N)c3ccc2n1Cc1ccc(-c2ccccc2C#N)cc1]

### 4′-((2,4-diamino-8-methyl-7H-pyrrolo[2,3-h]quinazolin-7-yl)methyl)-[1,1′-biphenyl]-2-carbonitrile

White powder (46 mg, 23% yield) was obtained from 8-methyl-7H-pyrrolo[2,3-h]quinazoline-2,4-diamine (100 mg, 0.5 mmol). ¹H NMR (300 MHz, DMSO-d₆) δ 7.92 (m, 1H), 7.82 (m, 1H), 7.74 (m, 2H), 7.50-7.59 (m, 5H), 7.12 (d, J=8.4 Hz, 1H), 6.80 (s, 1H), 5.64 (s, 2H), 2.34 (s, 3H). MS (ESI): Calcd for C₂₅H₂₁N₆⁺ 405.17 [M+H]⁺, found 405.05 [M+H]⁺.

### Example 32. Preparation of 7-((2′-(1H-tetrazol-5-yl)-[1,1′-biphenyl]-4-yl)methyl)-8-methyl-7H-pyrrolo[2,3-h]quinazoline-2,4-diamine

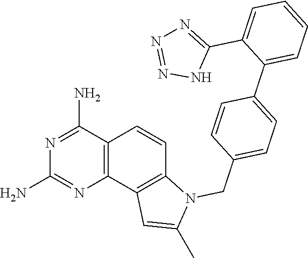 [SMILES: Cc1cc2c3nc(N)nc(N)c3ccc2n1Cc1ccc(-c2ccccc2-c2nnn[nH]2)cc1]

### 7-((2′-(1H-tetrazol-5-yl)-[1,1′-biphenyl]-4-yl)methyl)-8-methyl-7H-pyrrolo[2,3-h]quinazoline-2,4-diamine

To a solution of 8-methyl-7H-pyrrolo[2,3-h]quinazoline-2,4-diamine (100 mg, 0.5 mmol) in anhydrous DMF (0.5 mL) was added NaH (60% in mineral oil, 30 mg, 0.75 mmol) under nitrogen. After stirring at r.t. for 10 min, 5-(4′-(bromomethyl)-[1,1′-biphenyl]-2-yl)-1-trityl-1H-tetrazole (279 mg, 0.5 mmol) was added. The reaction mixture was stirred at r.t. overnight, then treated with water, the precipitate was filtered, washed with water, and dried. Then it was suspended in DCM and loaded on silica gel, purified by column chromatography on silica gel using 0-10% MeOH in DCM as eluents to give the intermediate which was purified by column chromatography on silica gel. Then it was dissolved in MeOH (5 mL) and treated with HCl solution (4 M in dioxane, 0.5 mL, 2 mmol). The mixture was stirred at r.t. for 4 hours and concentrated in vacuo. The residue was dissolved in MeOH and neutralized with DIPEA. After removing solvents, it was purified by column chromatography on silica gel using MeOH in DCM as eluents to give a w % bite powder (43 mg, 19% yield). ¹H NMR (300 MHz, DMSO-d₆) δ 7.82 (m, 1H), 7.57 (m, 2H), 7.52 (m, 1H), 7.49 (m, 1H), 7.43 (m, 1H), 7.19 (m, 2H), 7.04 (d, J=8.4 Hz, 2H), 6.90 (s, 1H), 5.52 (s, 2H), 2.37 (s, 3H). MS (ESI): Calcd for C₂₅H₂₂N₉⁺ 448.19 [M+H]⁺, found 448.05 [M+H]⁺.

### Example 33. Preparation of 7-(benzo[d]thiazol-2-ylmethyl)-8-methyl-7H-pyrrolo[2,3-h]quinazoline-2,4-diamine

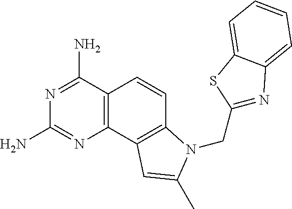 [SMILES: Cc1cc2c3nc(N)nc(N)c3ccc2n1Cc1nc2ccccc2s1]

### 7-(benzo[d]thiazol-2-ylmethyl)-8-methyl-7H-pyrrolo[2,3-h]quinazoline-2,4-diamine

Pale brown powder (45 mg, 25% yield) was obtained from 8-methyl-7H-pyrrolo[2,3-h]quinazoline-2,4-diamine (100 mg, 0.5 mmol). MS (ESI): Calcd for C₁₉H₁₇N₆S⁺ 361.12 [M+H]⁺, found 360.90 [M+H]⁺.

### Example 34. Preparation of 7-(5-bromo-2-fluorobenzyl)-7H-pyrrolo[2,3-h]quinazoline-2,4-diamine

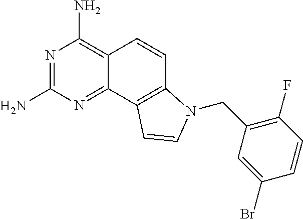 [SMILES: Nc1nc(N)c2ccc3c(ccn3Cc3cc(Br)ccc3F)c2n1]

### 7-(5-bromo-2-fluorobenzyl)-7H-pyrrolo[2,3-h]quinazoline-2,4-diamine

Off white powder (10 mg, 13% yield) was obtained from 7H-pyrrolo[2,3-h]quinazoline-2,4-diamine (40 mg, 0.20 mmol). ¹H NMR (300 MHz, CD₃OD) δ 7.66 (d, J=9.3 Hz, 1H) 7.44 (m, 1H), 7.36 (d, J=3.3 Hz, 1H), 7.29 (d, J=8.7 Hz, 1H), 7.12-7.03 (m, 3H), 5.50 (s, 2H). MS (ESI): Calcd for C₁₇H₁₄BrFN₅⁺ 386.04 [M+H]⁺, found 385.95 [M+H]⁺.

### Example 35. Preparation of 7-(2,4-dibromobenzyl)-7H-pyrrolo[2,3-h]quinazoline-2,4-diamine

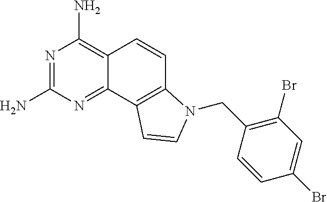 [SMILES: Nc1nc(N)c2ccc3c(ccn3Cc3ccc(Br)cc3Br)c2n1]

### 7-(2,4-dibromobenzyl)-7H-pyrrolo[2,3-h]quinazoline-2,4-diamine

Beige powder (29 mg, 32% yield) was obtained from 7H-pyrrolo[2,3-h]quinazoline-2,4-diamine (40 mg, 0.20 mmol). ¹H NMR (300 MHz, CD₃OD) δ 7.83 (s, 1H), 7.59 (d, J=10.2 Hz, 1H), 7.34 (d, J=8.4 Hz, 1H), 7.26 (m, 1H), 7.08-7.05 (m, 2H), 6.43 (d, J=7.8 Hz, 1H), 5.45 (s, 2H). MS (ESI): Calcd for C₁₇H₁₄Br₂N₅⁺ 447.96 [M+H]⁺, found 447.90 [M+H]⁺.

### Example 36. Preparation of 7-((2′-(trifluoromethyl)-[1,1′-biphenyl]-4-yl)methyl)-7H-pyrrolo[2,3-h]quinazoline-2,4-diamine

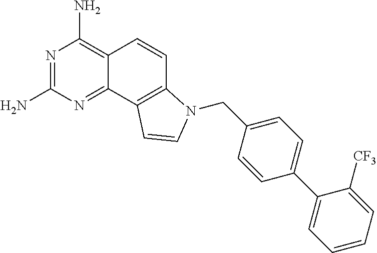 [SMILES: Nc1nc(N)c2ccc3c(ccn3Cc3ccc(-c4ccccc4C(F)(F)F)cc3)c2n1]

### 7-((2′-(trifluoromethyl)-[1,1′-biphenyl]-4-yl)methyl)-7H-pyrrolo[2,3-h]quinazoline-2,4-diamine

Beige powder (12 mg, 14% yield) was obtained from 7H-pyrrolo[2,3-h]quinazoline-2,4-diamine (40 mg, 0.20 mmol). ¹H NMR (300 MHz, CD₃OD) δ 7.74 (d, J=7.5 Hz, 1H), 7.67-7.57 (m, 2H), 7.50 (m, 1H), 7.38 (d, J=3.0 Hz, 1H), 7.31-7.17 (m, 6H), 7.05 (d, J=3.0 Hz, 1H), 5.53 (s, 2H). MS (ESI): Calcd for C₂₄H₁₉F₃N₅⁺ 434.16 [M+H]⁺, found 434.05 [M+H]⁺.

### Example 37. Preparation of 4′-((2,4-diamino-7H-pyrrolo[2,3-h]quinazolin-7-yl)methyl)-4-fluoro-[1,1′-biphenyl]-2-carbonitrile

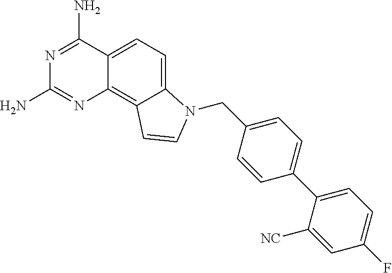 [SMILES: N#Cc1cc(F)ccc1-c1ccc(Cn2ccc3c4nc(N)nc(N)c4ccc32)cc1]

### 4′((2,4-diamino-7H-pyrrolo[2,3-h]quinazolin-7-yl)methyl)-4-fluoro-[1,1′-biphenyl]-2-carbonitrile

White powder (24 mg, 29% yield) was obtained from 7H-pyrrolo[2,3-h]quinazoline-2,4-diamine (40 mg, 0.20 mmol). ¹H NMR (300 MHz, DMSO-d₆) δ 7.94 (m, 1H), 7.62 (m, 3H), 7.47 (m, 3H), 7.31-7.22 (m, 3H), 7.04 (s br, 2H), 6.81 (d, J=2.4 Hz, 1H), 6.00 (s br, 2H), 5.54 (s, 2H). MS (ESI): Calcd for C₂₄H₁₈FN₆⁺ 409.16 [M+H]⁺, found 409.05 [M+H]⁺.

### Example 38. Preparation of 4′-((2,4-diamino-7H-pyrrolo[2,3-h]quinazolin-7-yl)methyl)-2′-fluoro-[1,1′-biphenyl]-2-carbonitrile

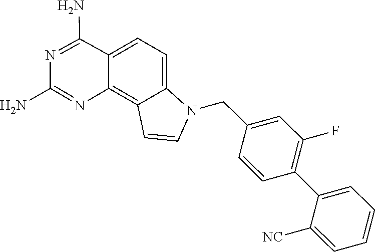 [SMILES: N#Cc1ccccc1-c1ccc(Cn2ccc3c4nc(N)nc(N)c4ccc32)cc1F]

### 4′-((2,4-diamino-7H-pyrrolo[2,3-h]quinazolin-7-yl)methyl)-2′-fluoro-[1,1′-biphenyl]-2-carbonitrile

Off white powder (35 mg, 43% yield) was obtained from 7H-pyrrolo[2,3-h]quinazoline-2,4-diamine (40 mg, 0.20 mmol). ¹H NMR (300 MHz, DMSO-d₆) δ 7.94 (d, J=7.5 Hz, 1H), 7.77 (m, 1H), 7.66-7.53 (m, 3H), 7.46-7.40 (m, 2H), 7.26-7.17 (m, 2H), 7.09 (d, J=7.8 Hz, 1H), 7.00 (s br, 2H), 6.82 (d, J=2.7 Hz, 1H), 5.95 (s br, 2H), 5.56 (s, 2H). MS (ESI): Calcd for C₂₄H₁₈FN₆⁺ 409.16 [M+H]⁺, found 409.00 [M+H]⁺.

### Example 39. Preparation of 4′-((2,4-diamino-7H-pyrrolo[2,3-h]quinazolin-7-yl)methyl)-3′-fluoro-[1,1′-biphenyl]-2-carbonitrile

6)

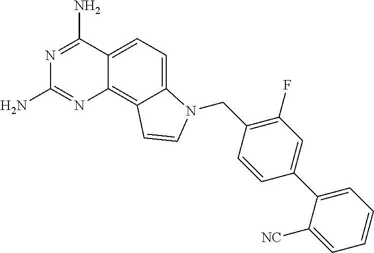 [SMILES: N#Cc1ccccc1-c1ccc(Cn2ccc3c4nc(N)nc(N)c4ccc32)c(F)c1]

### 4′-((2,4-diamino-7H-pyrrolo[2,3-h]quinazolin-7-yl)methyl)-3′-fluoro-[1,1′-biphenyl]-2-carbonitrile

White powder (32 mg, 39% yield) was obtained from 7H-pyrrolo[2,3-h]quinazoline-2,4-diamine (40 mg, 0.20 mmol). ¹H NMR (30) MHz, DMSO-d₆) δ 7.60 (s, 2H), 7.56 (d, J=7.8 Hz, 1H), 7.46 (d, J=9.0 Hz, 1H), 7.40 (m, 1H), 7.27-7.10 (m, 4H), 7.46 (d, J=9.0 Hz, 1H), 6.99 (d, J=8.1 Hz, 2H), 6.76 (m, 1H), 6.62 (d, J=3.0 Hz, 1H), 5.89 (s br, 2H), 5.29 (s, 2H). MS (ESI): Calcd for C₂₄H₁₈FN₆⁺ 409.15 [M+H]⁺, found 409.00 [M+H]⁺.

### Example 40. Preparation of 4′-((2,4-diamino-7H-pyrrolo[2,3-h]quinazolin-7-yl)methyl)-2′,3′-difluoro-[1,1′-biphenyl]-2-carbonitrile

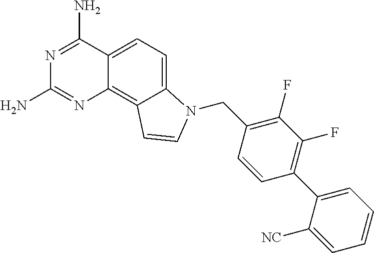 [SMILES: N#Cc1ccccc1-c1ccc(Cn2ccc3c4nc(N)nc(N)c4ccc32)c(F)c1F]

### 4′-((2,4-diamino-7H-pyrrolo[2,3-h]quinazolin-7-yl)methyl)-2′,3′-difluoro-[1,1′-biphenyl]-2-carbonitrile

Yellow powder (20 mg, 23% yield) was obtained from 7H-pyrrolo[2,3-h]quinazoline-2,4-diamine (40 mg, 0.20 mmol). ¹H NMR (300 MHz, DMSO-do) S 7.60 (m, 3H), 7.45 (m, 1H), 7.33-7.19 (m, 4H), 6.92 (m, 1H), 6.59 (m, 1H), 5.43 (s, 2H). MS (ESI): Calcd for C₂₄H₁₇F₂N₆⁺ 427.15 [M+H]⁺, found 427.05 [M+H]⁺.

### Example 41. Preparation of 7-(3-phenylprop-2-yn-1-yl)-7H-pyrrolo[2,3-h]quinazoline-2,4-diamine

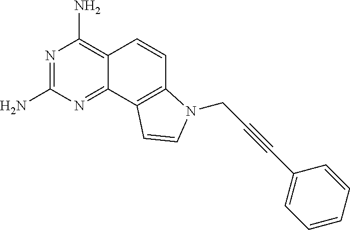 [SMILES: Nc1nc(N)c2ccc3c(ccn3CC#Cc3ccccc3)c2n1]

### 7-(3-phenylprop-2-yn-1-yl)-7H-pyrrolo[2,3-h]quinazoline-2,4-diamine

Off white powder (12 mg, 19% yield) was obtained from 7H-pyrrolo[2,3-h]quinazoline-2,4-diamine (40 mg, 0.20 mmol). ¹H NMR (300 MHz, DMSO) 7.75 (d, J=8.7 Hz, 1H), 7.50-7.28 (m, 9H), 6.83 (d, J=3.6 Hz, 1H), 6.28 (s br, 2H), 5.39 (s, 2H). MS (ESI): Calcd for C₁₉H₁₆N₅⁺ 314.14 [M+H]⁺, found 314.00 [M+H]⁺.

### Example 42. Preparation of 7-(4-ethynylbenzyl)-7H-pyrrolo[2,3-h]quinazoline-2,4-diamine

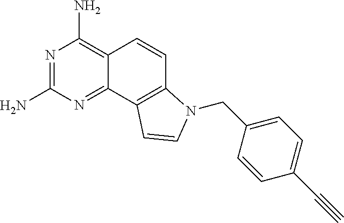 [SMILES: C#Cc1ccc(Cn2ccc3c4nc(N)nc(N)c4ccc32)cc1]

### 7-(4-ethynylbenzyl)-7H-pyrrolo[2,3-h]quinazoline-2,4-diamine

Beige powder (16 mg, 25% yield) was obtained from 7H-pyrrolo[2,3-h]quinazoline-2,4-diamine (40 mg, 0.20 mmol). ¹H NMR (300 MHz, DMSO-d₆) δ 7.62 (d, J=9.3 Hz, 1H), 7.39 (d, J=7.2 Hz, 2H), 7.14 (d, J=7.8 Hz, 2H), 7.08 (s br, 2H), 6.80 (s, 1H), 6.02 (s br, 2H), 5.46 (s, 2H), 4.14 (s, 1H). MS (ESI): Calcd for C₁₉H₁₆N₅⁺ 314.14 [M+H]⁺, found 314.00 [M+H]⁺.

### Example 43. Preparation of 7-(4-(phenylethynyl)benzyl)-7H-pyrrolo[2,3-h]quinazoline-2,4-diamine

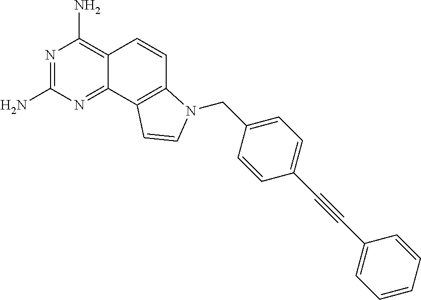 [SMILES: Nc1nc(N)c2ccc3c(ccn3Cc3ccc(C#Cc4ccccc4)cc3)c2n1]

### 7-(4-(phenylethynyl)benzyl)-7H-pyrrolo[2,3-h]quinazoline-2,4-diamine

White powder (25 mg, 32% yield) was obtained from 7H-pyrrolo[2,3-h]quinazoline-2,4-diamine (40 mg, 0.20 mmol). ¹H NMR (300 MHz, DMSO-d₆) δ 7.77 (d, J=7.5 Hz, 1H), 7.58 (s, 1H), 7.54-7.35 (m, 9H), 7.20 (m, 1H), 6.98 (s, 1H), 5.54 (s, 2H). MS (ESI): Calcd for C₂₅H₂₀N₅⁺ 390.16 [M+H]⁺, found 390.00 [M+H]⁺.

### Example 44. Preparation of 7-(3-(phenylethynyl)benzyl)-7H-pyrrolo[2,3-h]quinazoline-2,4-diamine

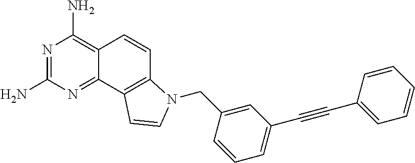 [SMILES: Nc1nc(N)c2ccc3c(ccn3Cc3cccc(C#Cc4ccccc4)c3)c2n1]

### 7-(3-(phenylethynyl)benzyl)-7H-pyrrolo[2,3-h]quinazoline-2,4-diamine

White powder (20 mg, 26% yield) was obtained from 7H-pyrrolo[2,3-h]quinazoline-2,4-diamine (40 mg, 0.20 mmol). ¹H NMR (300 MHz, DMSO-d₆) δ 7.83 (d, J=9.3 Hz, 1H), 7.69 (s, 1H), 7.61-7.30 (m, 9H), 7.25 (m, 1H), 7.02 (s, 1H), 5.55 (s, 2H). MS (ESI): Calcd for C₂₅H₂₀N₅⁺ 390.16 [M+H]⁺, found 390.05 [M+H]⁺.

### Example 45. Preparation of 7-(3-((2-(trifluoromethyl)phenyl)ethynyl)benzyl)-7H-pyrrolo[2,3-h]quinazoline-2,4-diamine

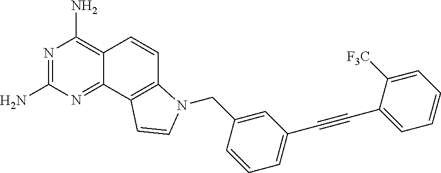 [SMILES: Nc1nc(N)c2ccc3c(ccn3Cc3cccc(C#Cc4ccccc4C(F)(F)F)c3)c2n1]

### 7-(3-((2-(trifluoromethyl)phenyl)ethynyl) benzyl)-7H-pyrrolo[2,3-h]quinazoline-2,4-diamine

Off white powder (26 mg, 28% yield) was obtained from 7H-pyrrolo[2,3-h]quinazoline-2,4-diamine (40 mg, 0.20 mmol). ¹H NMR (300 MHz, DMSO) δ 7.82-7-75 (m, 2H), 7.71-7.64 (m, 2H), 7.59 (m, 1H), 7.46-7.34 (m, 4H), 7.24 (m, 2H), 6.84 (s, 1H), 5.51 (s, 2H). MS (ESI): Calcd for C₂₆H₁₉F₃N₅⁺ 458.15 [M+H]⁺, found 458.05 [M+H]⁺.

### Example 46. Preparation of 4′-((2,4-diamino-5-bromo-7H-pyrrolo[2,3-h]quinazolin-7-yl)methyl)-2′-fluoro-[1,1′-biphenyl]-2-carbonitrile

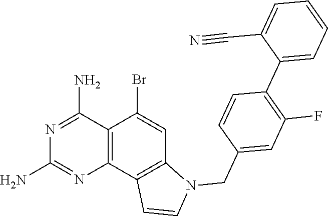 [SMILES: N#Cc1ccccc1-c1ccc(Cn2ccc3c4nc(N)nc(N)c4c(Br)cc32)cc1F]

### 4′-((2,4-diamino-5-bromo-7H-pyrrolo[2,3-h]quinazolin-7-yl)methyl)-2′-fluoro-[1,1′-biphenyl]-2-carbonitrile

White powder (26 mg, 53% yield) was obtained from 5-bromo-7H-pyrrolo[2,3-h]quinazoline-2,4-diamine (28 mg, 0.1 mmol). ¹H NMR (300 MHz, CDCl₃) δ 7.76 (d, J=8.1 Hz, 1H), 7.65 (dd, J=8.1, 1.5 Hz, 1H), 7.84 (t, J=7.8 Hz, 2H), 7.37 (t, J=7.8 Hz, 1H), 7.35 (s, 1H), 7.13 (m, 2H), 6.93 (d, J=7.8 Hz, 1H), 6.86 (d, J=14.4 Hz, 1H), 5.38 (s, 2H), 4.87 (s br, 2H). MS (ESI): Calcd for C₂₄H₁₇BrFN₆⁺ 487.07 [M+H]⁺, found 487.00 [M+H]⁺.

### Example 47. Preparation of 5-bromo-7-(3-bromobenzyl)-7H-pyrrolo[2,3-h]quinazoline-2,4-diamine

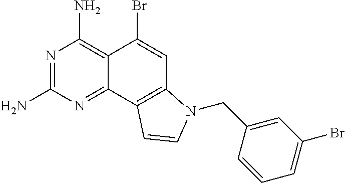 [SMILES: Nc1nc(N)c2c(Br)cc3c(ccn3Cc3cccc(Br)c3)c2n1]

### 5-bromo-7-(3-bromobenzyl)-7H-pyrrolo[2,3-h]quinazoline-2,4-diamine

Off-white powder (18 mg, 40% yield) was obtained from 5-bromo-7H-pyrrolo[2,3-h]quinazoline-2,4-diamine (28 mg, 0.1 mmol). ¹H NMR (300 MHz, MeOH-d₄) δ 7.55 (d, J=0.9 Hz, 1H), 7.43 (m, 1H), 7.33 (d, J=3.2 Hz, 1H), 7.32 (m, 1H), 7.23 (t, J=8.0 Hz, 1H), 7.09 (m, 1H), 7.02 (dd, J=3.0, 0.8 Hz, 1H), 5.42 (s, 2H). MS (ESI): Calcd for C₁₇H₁₄Br₂N₅⁺ 447.96 [M+H]⁺, found 447.85 [M+H]⁺.

### Example 48. Preparation of 5-bromo-N²,7-bis(3-bromobenzyl)-7H-pyrrolo[2,3-h]quinazoline-2,4-diamine

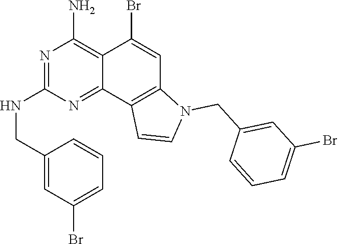 [SMILES: Nc1nc(NCc2cccc(Br)c2)nc2c1c(Br)cc1c2ccn1Cc1cccc(Br)c1]

### 5-bromo-N²,7-bis(3-bromobenzyl)-7H-pyrrolo[2,3-h]quinazoline-2,4-diamine

Brown powder (9 mg, 15% yield) was obtained from 5-bromo-7H-pyrrolo[2,3-h]quinazoline-2,4-diamine (28 mg, 0.1 mmol) as a byproduct. ¹H NMR (300 MHz, CDCl₃) δ 7.57 (s, 1H), 7.42 (m, 1H), 7.39 (m, 1H), 7.35 (m, 1H), 7.27 (s, 1H), 7.20 (m, 2H), 7.18 (m, 1H), 7.13 (m, 1H), 7.10 (d, J=3.2 Hz, 1H), 7.93 (m, 1H), 5.28 (s, 2H), 4.71 (s, 2H). MS (ESI): Calcd for C₂₄H₁₉Br₃N₅⁺ 615.92 [M+H]⁺, found 615.85 [M+H]⁺.

### Example 49. Preparation of 4′-((2,4-diamino-7H-pyrrolo[3′,2′:5,6]pyrido[4,3-d]pyrimidin-7-yl)methyl)-[1,1′-biphenyl]-2-carbonitrile

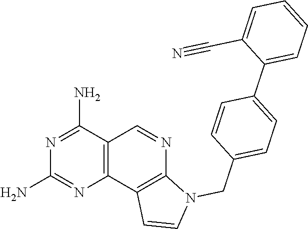 [SMILES: N#Cc1ccccc1-c1ccc(Cn2ccc3c4nc(N)nc(N)c4cnc32)cc1]

### 4′-((2,4-diamino-7H-pyrrolo[3′,2′:5,6]pyrido[4,3-d]pyrimidin-7-yl)methyl)-[1,1′-biphenyl]-2-carbonitrile

White powder (42 mg, 40% yield) was obtained from 4-chloro-1-((2′-cyano-[1,1′-biphenyl]-4-yl)methyl)-1H-pyrrolo[2,3-b]pyridine-5-carbonitrile (100 mg, 0.27 mmol) according to the general synthesis as described for intermediate D. ¹H NMR (300 MHz, DMSO-d₆) δ 8.87 (s, 1H), 7.90 (d, J=8.0 Hz, 1H), 7.75 (t, J=8.0 Hz, 2H), 7.55 (d, J=7.8 Hz, 2H), 7.49 (m, 2H), 7.13 (m, 2H), 6.70 (s, 1H), 6.57 (s br, 2H), 5.57 (s, 2H). MS (ESI): Calcd for C₂₃H₁₈N₇⁺ 392.16 [M+H]⁺, found 392.05 [M+H]⁺.

The requisite intermediates were prepared as follows:

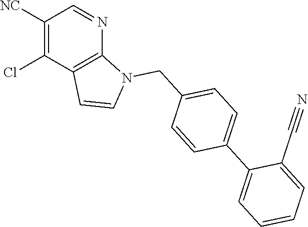 [SMILES: N#Cc1ccccc1-c1ccc(Cn2ccc3c(Cl)c(C#N)cnc32)cc1]

### 4-chloro-1-((2′-cyano-[1,1′-biphenyl]-4-yl)methyl)-1H-pyrrolo[2,3-b]pyridine-5-carbonitrile

To a solution of 4-chloro-1H-pyrrolo[2,3-b]pyridine-5-carbonitrile (53 mg, 0.3 mmol) in anhydrous DMF (0.5 mL) was added NaH (60% in mineral oil, 18 mg, 0.45 mmol) under nitrogen. After stirring at r.t. for 10 min, 4′-(bromomethyl)-[1,1′-biphenyl]-2-carbonitrile (82 mg, 0.3 mmol) was added. The reaction mixture was stirred at r.t. for 3 hours, then treated with water and extracted with DCM, washed with water, brine and dried. After concentration it was purified by column chromatography on silica gel using EtOAc in hexane as eluents to give the product as a white powder (100 mg, 90% yield). ¹H NMR (300 MHz, CDC₃) δ 8.55 (s, 1H), 7.76 (d, J=7.8 Hz, 1H), 7.64 (t, J=8.0 Hz, 1H), 7.52 (d, J=8.0 Hz, 2H), 7.46 (d, J=7.8 Hz, 2H), 7.41 (d, J=3.6 Hz, 1H), 7.32 (d, J=8.1 Hz, 2H), 6.73 (d, J=3.6 Hz, 1H), 5.57 (s, 2H).

### Example 50. Preparation of 7-(3-bromobenzyl)-7H-pyrrolo[3′,2′:5,6]pyrido[4,3-d]pyrimidine-2,4-diamine

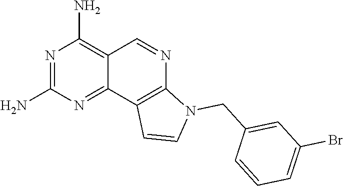 [SMILES: Nc1nc(N)c2cnc3c(ccn3Cc3cccc(Br)c3)c2n1]

### 7-(3-bromobenzyl)-7H-pyrrolo[3′,2′:5,6]pyrido[4,3-d]pyrimidine-2,4-diamine

Off white powder (18 mg, 24% yield) was obtained from 7H-pyrrolo[3′,2′:5,6]pyrido[4,3-d]pyrimidine-2,4-diamine (40 mg, 0.2 mmol). ¹H NMR (300 MHz, MeOH-d₄) δ 8.87 (s, 1H), 7.41 (m, 1H), 7.37 (m, 2H), 7.22 (m, 1H), 7.17 (m, 1H), 6.94 (d, J=3.5 Hz, 1H), 5.54 (s, 2H). MS (ESI): Calcd for C₁₆H₁₄BrN₆⁺ 369.05 [M+H]⁺, found 368.90 [M+H]⁺.

### Example 51. Preparation of 4′-((2,4-diamino-7H-pyrrolo[3′,2′:5,6]pyrido[4,3-d]pyrimidin-7-yl)methyl)-2′-fluoro-[1,1′-biphenyl]-2-carbonitrile

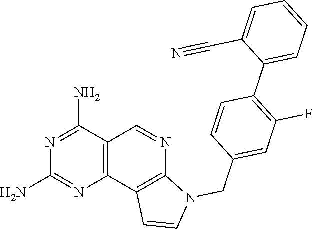 [SMILES: N#Cc1ccccc1-c1ccc(Cn2ccc3c4nc(N)nc(N)c4cnc32)cc1F]

### 4′-((2,4-diamino-7H-pyrrolo[3′,2′:5,6]pyrido[4,3-d]pyrimidin-7-yl)methyl)-2′-fluoro-[1,1′-biphenyl]-2-carbonitrile

White powder (21 mg, 35% yield) was obtained from 7H-pyrrolo[3′,2′:5,6]pyrido[4,3-d]pyrimidine-2,4-diamine (30 mg, 0.15 mmol). ¹H NMR (30) MHz, MeOH-d₄) δ 8.87 (s, 1H), 7.82 (dd, J=7.8, 0.9 Hz, 1H), 7.71 (td, J=8., 1.4 Hz, 1H), 7.56 (td, J=7.8, 1.3 Hz, 1H), 7.50 (d, J=8.1 Hz, 1H), 7.42 (d, J=3.6 Hz, 1H), 7.36 (d, J=7.8 Hz, 1H), 7.10 (m, 2H), 6.98 (d, J=3.4 Hz, 1H), 5.64 (s, 2H). MS (ESI): Calcd for C₂₃H₁₇FN₇⁺ 410.15 [M+H]⁺, found 410.05 [M+H]⁺.

### Example 52. Preparation of 4′-((4-amino-2-(((2′-cyano-2-fluoro-[1,1′-biphenyl]-4-yl)methyl)amino)-7H-pyrrolo[3′,2′:5,6]pyrido[4,3-d]pyrimidin-7-yl)methyl)-2′-fluoro-[1,1′-biphenyl]-2-carbonitrile

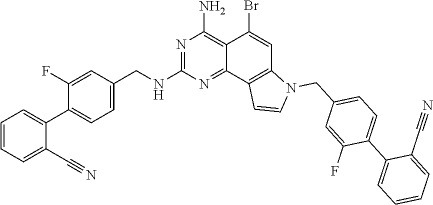 [SMILES: N#Cc1ccccc1-c1ccc(CNc2nc(N)c3c(Br)cc4c(ccn4Cc4ccc(-c5ccccc5C#N)c(F)c4)c3n2)cc1F]

### 4′-((4-amino-2-(((2′-cyano-2-fluoro-[1,1′-biphenyl]-4-yl)methyl)amino)-7H-pyrrolo[3′,2′:5,6]pyrido[4,3-d]pyrimidin-7-yl)methyl)-2′-fluoro-[1,1′-biphenyl]-2-carbonitrile

Brown powder (10 mg, 11% yield) was obtained from 7H-pyrrolo[3′,2′:5,6]pyrido[4,3-d]pyrimidine-2,4-diamine (30 mg, 0.15 mmol) as a byproduct. ¹H NMR (300 MHz, CDCl₃) δ 8.75 (s, 1H), 7.75 (m, 3H), 7.61 (m, 3H), 7.45 (m, 5H), 7.32 (m, 1H), 7.17 (m, 1H), 7.05 (m, 2H), 6.95 (m, 1H), 5.55 (s, 2H), 4.84 (s, 2H). MS (ESI): Calcd for C₃₇H₂₅F₂N₈⁺ 619.22 [M+H]⁺, found 619.20 [M+H]⁺.

### Example 53. Preparation of 7-(4-ethynylbenzyl)-7H-pyrrolo[3′,2′:5,6]pyrido[4,3-d]pyrimidine-2,4-diamine

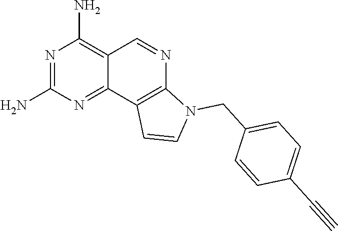 [SMILES: C#Cc1ccc(Cn2ccc3c4nc(N)nc(N)c4cnc32)cc1]

### 7-(4-ethynylbenzyl)-7H-pyrrolo[3′,2′:5,6]pyrido[4,3-d]pyrimidine-2,4-diamine

White powder (21 mg, 45% yield) was obtained from 7H-pyrrolo[3′,2′:5,6]pyrido[4,3-d]pyrimidine-2,4-diamine (30 mg, 0.15 mmol). ¹H NMR (300 MHz, MeOH-d₄) δ 8.84 (s, 1H), 7.38 (d, J=8.4 Hz, 2H), 7.32 (d, J=3.6 Hz, 1H), 7.15 (d, J=8.4 Hz, 2H), 6.93 (d, J=3.6 Hz, 1H), 5.55 (s, 2H), 3.10 (s, 1H). MS (ESI): Calcd for C₁₈H₁₅N₆⁺ 315.14 [M+H]⁺, found 315.00 [M+H]⁺.

### Example 54: MIC Assays

MIC assays for N. gonorrhoeae were done using the ATCC 49226 strain. The procedure guidelines recommended by the Clinical and Laboratory Standards Institute (CLSI) for the broth microdilution MIC assay were modified by substituting cation-adjusted Mueller-Hinton (CAMH) broth for Todd-Hewitt (TH) broth. Also, the recommended bacterial inoculum was modified to meet the high inoculum demands needed for N. gonorrhoeae growth. A 96-well plate containing TH broth with 2-fold serial dilution of compounds was inoculated with log-phase bacterial at 2.5×10⁷ CFU/mL. The final volume in each well was 200 μL. Each compound was tested in duplicate. The microtiter plates were incubated in an anaerobic environment for 24 hours at 37° C. using the candle jar technique. Then the bacterial growth was tested by reading the plate with a SpectraMax iD5 plate reader (Molecular Devices, Inc.) at 600 nm. The MIC was defined as the lowest compound concentration that inhibited bacteria growth.

MIC assays for various Gram-negative and Gram-positive strains were conducted in accordance with the CLSI guidelines for broth microdilution. A 96-well plate containing CAMH broth with 2-fold serial dilution of compounds was inoculated with log-phase bacterial at 5×10⁵ CFU/mL. The final volume in each well was 100 μL. Each compound was tested in duplicate. The microtiter plates were incubated in an aerobic environment for 18 hours at 37° C. Then the bacterial growth was tested by reading the plate with a SpectraMax iD5 plate reader (Molecular Devices, Inc.) at 600 nm. The MIC was defined as the lowest compound concentration that inhibited bacteria growth.

The following Gram-negative and Gram-positive strains were used in the aerobic MIC procedure:
P. aeruginosa ATCC 27853
A. baumannii ATCC 19606
E. coli ATCC 25922
E. coli W4573
E. coli N43
K. pneumoniae ATCC 10031
K. pneumoniae ATCC 13883
S. sonnei ATCC 29930
P. mirabilis ATCC 29906
Methicillin-susceptible S. aureus (MSSA) ATCC 19636
Methicillin-resistant S. aureus (MRSA) ATCC 33951

### Example 55

The following can illustrate representative pharmaceutical dosage forms, containing a compound of formula I (‘Compound X’) or a pharmaceutically acceptable salt thereof, for therapeutic or prophylactic use in humans. The tablets can optionally comprise an enteric coating.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 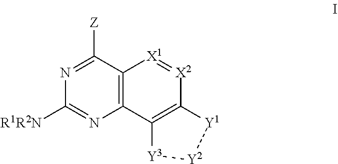
wherein;
R¹ is hydrogen, (C₁-C₆)alkyl, (C₂-C₆)alkenyl, (C₂-C₆)alkynyl, —C(═O)R^{a}, —C(═O)N(R^{a})₂, —S(═O)₂N(R^{a})₂, —C(═NR^{a})N(R^{a})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₂-C₆)alkenyl, (C₂-C₆)alkynyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, phenyl, and —OP(═O)(OH)₂, wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy; and
R² is hydrogen, (C₁-C₆)alkyl, (C₂-C₆)alkenyl, —C(═O)R^{b}, —C(═O)N(R^{b})₂, —S(═O)₂N(R^{b}), —C(═NR^{b})N(R^{b})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₂-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{b})₂, —NR^{b}C(═NR^{b})N(R)₂, —C(═NR^{b})N(R^{b})₂, and —OP(═O)(OH)₂; or
R¹ and R² together with the nitrogen to which they are attached form a heterocyclyl, wherein the heterocycly is optionally substituted with one or more (e.g., 1, 2, 3, 4, or 5) groups independently selected from the group consisting of halo, —OH, —NO₂, —CN, (C₁-C₄)alkyl, (C₁—C₄)haloalkyl, (C₁-C₄)alkoxy, (C₁-C₄)haloalkoxy, —N(R^{ab})₂, —NR^{ab}C(═NR^{ab})N(R^{ab})₂, —C(═NR^{ab})N(R^{ab})₂, and —OP(═O)(OH)₂,
Z is —OR^{c}, —N(R^{c})₂, —SR^{c}, —NR^{c}C(═NR^{c})N(R^{c})₂—C(═NR^{c})N(R^{c})₂, hydrogen, (C₁-C₆)alkyl, (C₂-C₆)alkenyl, —C(═O)R^{c}, —C(═O)N(R^{c})₂, —S(═O)₂N(R^{c})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₂-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{c})₂, —NR^{c}C(═NR^{c})N(R^{c})₂, —C(═NR^{c})N(R^{c})₂, and —OP(═O)(OH)₂;
X¹ is CR³ or N;
X² is CR⁴ or N;
R³ is hydrogen, halo, —OH, —NO₂, —CN, (C₁-C₆)alkoxy, (C₂-C₆)alkyl, (C₂-C₆)alkenyl, (C₂-C₆)alkynyl, —C(═O)R^{d}, —C(═O)N(R^{d})₂, —S(═O)₂N(R^{d})₂, —NR^{d}C(═NR^{d})N(R^{d})₂, —C(═NR^{d})N(R^{d})₂, or —N(R^{d})₂, wherein the (C₁-C₆)alkoxy, (C₁-C₆)alkyl, (C₂-C₆)alkenyl, or (C₂-C₆)alkynyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{d})₂, —NR^{d}C(═NR^{d})N(R^{d})₂, —C(═NR^{d})N(R^{d})₂, and —OP(═O)(OH)₂;
R⁴ is hydrogen, halo, —OH, —NO₂, —CN, (C₁-C₆)alkoxy, (C₁-C₆)alkyl, (C₂-C₆)alkenyl, (C₂-C₆)alkynyl, —C(═O)R^{e}, —C(═O)N(R^{e})₂, —S(═O)₂N(R^{e})₂, —NR^{e}C(═NR^{e})N(R^{e})₂, —C(═NR^{e})N(R^{e})₂, or —N(R^{e})₂, wherein the (C₁-C₆)alkoxy, (C₁-C₆)alkyl, (C₂-C₆)alkenyl, or (C₂-C₆)alkynyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{e})₂, —NR^{e}C(═NR^{e})N(R^{e})₂, —C(═NR^{e})N(R^{e})₂, and —OP(═O)(OH)₂;
Y¹ is NR^{5a}, Y² is N or CR^{6b}, Y³ is N or CR^{7b}, the dashed bond between Y¹ and Y² is a single bond, and the dashed bond between Y² and Y¹ is a double bond; or
Y³ is NR^{7a}, Y¹ is N or CR^{5b}, Y² is N or CR^{6b}, the dashed bond between Y¹ and Y² is a double bond, and the dashed bond between Y² and Y³ is a single bond;
R^{5a} and R^{7a}re each independently (C₁-C₁₀)alkyl, (C₂-C₁₀)alkenyl, —W—(C₃-C₇)carbocyclyl, —W-aryl, W-heterocyclyl, -or W-heteroaryl, wherein the (C₁-C₁₀)alkyl, (C₂-C₁₀)alkenyl, —W—(C₃-C₇)carbocyclyl, —W-aryl, W-heterocyclyl, —W-heteroaryl, is optionally substituted with one or more (e.g., 1, 2, 3, 4, or 5) groups independently selected from Q;
R^{5b}, R^{6b}, and R^{7b} are each independently hydrogen, halo, —OH, —NO₂, —CN, (C₁-C₆)alkoxy, (C₁-C₆)alkyl, (C₂-C₆)alkenyl, —C(═O)R^{f}, —C(═O)N(R^{f})₂, —S(═O)₂N(R^{f})₂, —NR^{f}C(═NR^{f})N(R^{f})₂, —C(═NR^{f})N(R^{f})₂, or —N(R^{f})₂, wherein the (C₁-C₆)alkoxy, (C₁-C₆)alkyl, or (C₂-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{f})₂, —NR^{f}C(═NR^{f})N(R^{f})₂, —C(═NR^{f})N(R^{f})₂, and —OP(═O)(OH)₂;
W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—;
each Q is independently halo, —OH, —NO₂, —CN, (C₁-C₆)alkyl, (C₂-C₆)alkenyl, (C₂-C₆)alkynyl, (C₁-C₆)haloalkyl, (C₁-C₆)alkoxy, (C₁-C₆)haloalkoxy, C(═O)R^{q}, —C(═O)N(R^{q})₂, —N(R^{q})₂, —S(═O)₂N(R^{q})₂, aryl, —O-aryl, heteroaryl, or —O-heteroaryl, wherein the (C₁-C₆)alkyl, (C₂-C₆)alkenyl, (C₂-C₆)alkynyl, (C₁-C₆)haloalkyl, (C₁-C₆)alkoxy, (C₁-C₆)haloalkoxy, aryl, -Oaryl, heteroaryl, or -Oheteroaryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R⁴, —C(═O)O(R^{q}), —C(═O)N(R^{q})₂, —S(═O)₂N(R^{q})₂, aryl and heteroaryl, wherein the aryl or heteroaryl is optionally substituted with one or more (e.g., 1, 2, 3, 4, or 5) Q¹;
each Q¹ is independently halo, —OH, —NO₂, —CN, (C₁-C₄)alkyl, (C₁-C₄)haloalkyl, (C₁-C₄)alkoxy, or (C₁-C₄)haloalkoxy;
each R^{a}, R^{b}, R^{ab}, R^{c}, R^{d}, R^{e}, and R^{f}, is independently hydrogen, (C₁-C₆)alkyl, (C₂-C₆)alkenyl, (C₁-C₆)haloalkyl, (C₃-C₇)carbocyclyl, or aryl;
each R^{w} is independently hydrogen or (C₁-C₆)alkyl;
R^{q} is hydrogen, (C₁-C₆)alkyl, (C₂-C₆)alkenyl, (C₁-C₆)haloalkyl (C₃-C₇)carbocyclyl or aryl, wherein the aryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q1}, —C(═O)O(R^{q1}), —C(═O)N(R^{q1})₂, and —S(═O)₂N(R^{q1})₂; and
each R^{q1} is independently hydrogen, (C₁-C₆)alkyl, (C₂-C₆)alkenyl, (C₁-C₆)haloalkyl, (C₃-C₇)carbocyclyl, or aryl;
or a salt thereof.

2. The compound of claim 1 wherein: R¹ is hydrogen, (C₁-C₆)alkyl, (C₁-C₆)alkenyl, —C(═O)R^{a}, —C(═O)N(R^{a})₂, —S(═O)₂N(R^{a})₂, —C(═NR^{a})N(R^{a})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, and —OP(═O)(OH)₂; and
R² is hydrogen, (C₁-C₆)alkyl, (C₁-C₆)alkenyl, —C(═O)R^{b}, —C(═O)N(R^{b})₂, —S(═O)₂N(R^{b})₂, —C(═NR^{b})N(R^{b})₂, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, wherein the (C₁-C₆)alkyl, (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl-, is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{b})₂, —NR^{b}C(═NR^{b})N(R^{b})₂, —C(═NR^{b})N(R^{b})₂, and —OP(═O)(OH)₂; or
R¹ and R² together with the nitrogen to which they are attached form a heterocyclyl, wherein the heterocycly is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{ab})₂, —NR^{ab}C(═NR^{ab})N(R^{ab})₂, —C(═NR^{ab})N(R^{ab})₂, and —OP(═O)(OH)₂,
Z is —OR^{c}, —N(R^{c})₂, —SR^{c}, —NR^{c}C(═NR^{c})N(R^{c})₂, —C(═NR^{c})N(R^{c})₂, hydrogen, (C₁-C₆)alkyl, (C₁-C₆)alkenyl, —C(═O)R^{c}, —C(═O)N(R^{c})₂, —S(═O)₂N(R^{c})₂, (C₃-C₇)carbocyclyl, aryl, heterocyclyl, heteroaryl, (C₃-C₇)carbocyclyl(C₁-C₆)alkyl-, aryl(C₁-C₆)alkyl-heterocyclyl(C₁-C₆)alkyl-, or heteroaryl (C₁-C₆)alkyl-, wherein the (C₁-C₆)alkyl, (C₁-C₆)alkenyl, (C₃-C₇)carbocyclyl, aryl, heterocyclyl, heteroaryl, (C₃-C₇)carbocyclyl(C₁-C₆)alkyl-, aryl(C₁-C₆)alkyl-, heterocyclyl(C₁-C₆)alkyl-, or heteroaryl(C₁-C₆)alkyl- is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{c})₂, —NR^{c}C(═NR^{c})N(R)₂, —C(═NR^{c})N(R^{c})₂, and —OP(═O)(OH)₂;
X¹ is CR³ or N;
X² is CR⁴ or N;
R³ is hydrogen, halo, —OH, —NO₂, —CN, (C₁-C₆)alkoxy, (C₁-C₆)alkyl, (C₁-C₆)alkenyl, —C(═O)R^{d}, —C(═O)N(R^{d})₂, —S(═O)₂N(R^{d})₂, —NR^{d}C(═NR^{d})N(R^{d})₂, —C(═NR^{d})N(R^{d})₂, or —N(R^{d})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{d})₂, —NR^{d}C(═NR^{d})N(R^{d})₂, —C(═NR^{d})N(R^{d})₂, and —OP(═O)(OH)₂;
R⁴ is hydrogen, halo, —OH, —NO₂, —CN, (C₁-C₆)alkoxy, (C₁-C₆)alkyl, (C₁-C₆)alkenyl, —C(═O)R^{c}, —C(═O)N(R^{e})₂, —S(═O)₂N(R^{e})₂, —NR^{e}C(═NR)N(R^{c})₂, —C(═NR^{c})N(R^{e})₂, or —N(R^{e})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{e})₂, —NR^{e}C(═NR^{e})N(R^{e})₂, —C(═NR^{e})N(R^{e})₂, and —OP(═O)(OH)₂;
Y¹ is NR^{5a}, Y² is N or CR^{6b}, Y² is N or CR^{7b}, the dashed bond between Y¹ and Y² is a single bond, and the dashed bond between Y² and Y³ is a double bond; or
Y³ is NR^{7a}, Y¹ is N or CR^{5b}, Y² is N or CR^{6b}, the dashed bond between Y¹ and Y² is a double bond, and the dashed bond between Y² and Y³ is a single bond;
R^{5a} and R^{7a} are each independently (C₁-C₁₀)alkyl, (C₁-C₁₀)alkenyl, —W—(C₃-C₇)carbocyclyl, —W-aryl, W-heterocyclyl, -or W-heteroaryl, wherein the (C₁-C₁₀)alkyl, (C₁-C₁₀)alkenyl, —W—(C₃-C₇)carbocyclyl, —W-aryl, W-heterocyclyl, —W-heteroaryl, is optionally substituted with one or more (e.g., 1, 2, 3, 4, or 5) groups independently selected from Q;
R^{5b}, R^{6b}, and R^{7b} are each independently hydrogen, halo, —OH, —NO₂, —CN, (C₁-C₆)alkoxy, (C₁-C₆)alkyl, (C₁-C₆)alkenyl, —C(═O)R^{f}, —C(═O)N(R^{f})₂, —S(═O)₂N(R^{f})₂, —NR^{f}C(═NR^{f})N(R^{f})₂, —C(═NR^{f})N(R^{f})₂, or —N(R^{f})₂, wherein the (C₁-C₆)alkoxy, (C₁-C₆)alkyl, or (C₁-C₆)alkenyl is optionally substituted with one or more (e.g., 1, 2, 3, 4, or 5) groups independently selected from the group consisting of halo, —OH, —NO₂, —CN, (C₁-C₄)alkoxy, (C₁-C₄)haloalkoxy, —N(R^{f})₂, —NR^{f}C(═NR^{f})N(R^{f})₂, —C(═NR^{f})N(R^{f})₂, and —OP(═O)(OH)₂;
W is absent or (C₁-C₁₀)alkyl, wherein the (C₁-C₁₀)alkyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—;
each Q is independently halo, —OH, —NO₂, —CN, (C₁-C₆)alkyl, (C₁-C₆)haloalkyl, (C₁-C₆)alkoxy, (C₁-C₆)haloalkoxy, C(═O)R^{q}, —C(═O)N(R^{q})₂, —N(R^{q})₂, —S(═O)₂N(R^{a})₂, aryl, —O-aryl, heteroaryl, or —O-heteroaryl, wherein the (C₁-C₆)alkyl, (C₁-C₆)haloalkyl, (C₁-C₆)alkoxy, (C₁-C₆)haloalkoxy, aryl, -Oaryl, heteroaryl, or -Oheteroaryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q}, —C(═O)O(R^{q}), —C(═O)N(R^{q})₂, —S(═O)₂N(R^{q})₂, aryl and heteroaryl, wherein the aryl or heteroaryl is optionally substituted with one or more (e.g., 1, 2, 3, 4, or 5) Q¹;
each Q¹ is independently halo, —OH, —NO₂, —CN, (C₁-C₄)alkyl, (C₁-C₄)haloalkyl, (C₁-C₄)alkoxy, or (C₁-C₄)haloalkoxy;
each R^{a}, R^{b}, R^{ab}, R^{c}, R^{d}, R^{e}, and R^{f}, is independently hydrogen, (C₁-C₆)alkyl, (C₁-C₆)alkenyl, (C₁-C₆)haloalkyl, (C₃-C₇)carbocyclyl, or aryl;
each R^{q} is independently hydrogen or (C₁-C₆)alkyl;
R^{q} is hydrogen, (C₁-C₆)alkyl, (C₁-C₆)alkenyl, (C₁-C₆)haloalkyl (C₃-C₇)carbocyclyl or aryl, wherein the aryl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, C(═O)R^{q1}, —C(═O)O(R^{q1}), —C(═O)N(R^{q1})₂, and —S(═O)₂N(R^{q1})₂; and
each R^{q1} is independently hydrogen, (C₁-C₆)alkyl, (C₁-C₆)alkenyl, (C₁-C₆)haloalkyl, (C₃-C₇)carbocyclyl, or aryl;
or a salt thereof.

3. The compound or salt of claim 1, wherein X¹ is CR³.

4. The compound or salt of any one of claims 1-3, wherein X² is CR⁴.

5. The compound or salt of any one of claims 1-3, wherein X² is N.

6. The compound or salt of claim 1, wherein the compound of formula I is a compound of formula Ie: 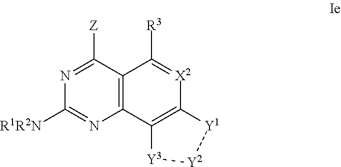
or a salt thereof.

7. The compound or salt of claim 1, wherein the compound of formula I is a compound of formula If: 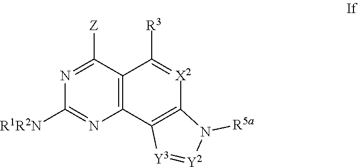
or a salt thereof.

8. The compound or salt of claim 1, wherein the compound of formula Ig is a compound of formula Ig: 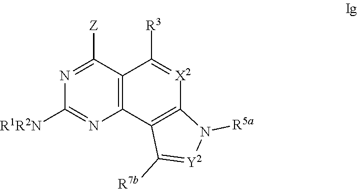
or a salt thereof.

9. The compound or salt of claim 1, wherein the compound of formula I is a compound of formula Ia: 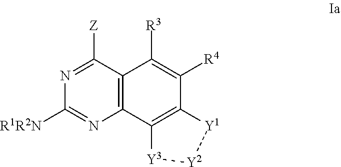
or a salt thereof.

10. The compound or salt of claim 1, wherein the compound of formula I is a compound of formula Ib: 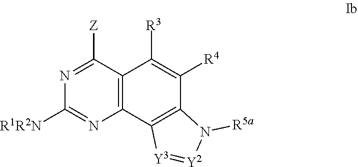
or a salt thereof.

11. The compound or salt of any one of claims 1-10, wherein Y³ is CR^{7b}.

12. The compound or salt of any one of claims 1-11, wherein Y² is CR^{6b}.

13. The compound or salt of claim 1, wherein the compound of formula I is a compound of formula Id: 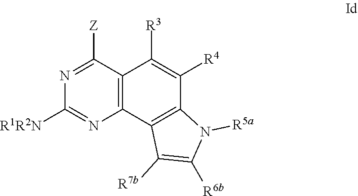
or a salt thereof.

14. The compound or salt of claim 1, wherein the compound of formula I is a compound of formula Ih: 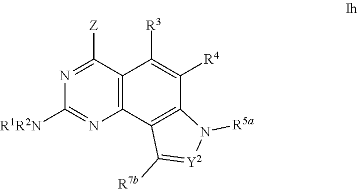
or a salt thereof.

15. The compound or salt of any one of claims 1-14, wherein R³ is hydrogen or halo.

16. The compound or salt of any one of claims 1-14, wherein R³ is hydrogen.

17. The compound or salt of any one of claims 1-16, wherein R⁴ is hydrogen.

18. The compound or salt of any one of claims 1-17, wherein R¹ is hydrogen, (C₁-C₆)alkyl, or aryl(C₁-C₆)alkyl-, wherein the (C₁-C₆)alkyl or aryl(C₁-C₆)alkyl is optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, —N(R^{a})₂, —NR^{a}C(═NR^{a})N(R^{a})₂, —C(═NR^{a})N(R^{a})₂, phenyl, and —OP(═O)(OH)₂; wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy.

19. The compound or salt of any one of claims 1-17, wherein R¹ is hydrogen, (C₁-C₆)alkyl, or aryl(C₁-C₆)alkyl-, wherein the (C₁-C₆)alkyl or aryl(C₁-C₆)alkyl is optionally substituted with one or more (e.g., 1, 2, 3, 4, or 5) groups independently selected from the group consisting of halo and phenyl, wherein phenyl is optionally substituted with one or more halo, —OH, —NO₂, —CN, (C₁-C₄)alkyl, (C₁-C₄)haloalkyl, (C₁-C₄)alkoxy, or (C₁-C₄)haloalkoxy.

20. The compound or salt of any one of claims 1-17, wherein R¹ is hydrogen.

21. The compound or salt of any one of claims 1-17, wherein R² is hydrogen.

22. The compound or salt of any one of claims 1-18, wherein Z is —N(R^{c})₂.

23. The compound or salt of any one of claims 1-18, wherein Z is —NH₂.

24. The compound or salt of any one of claims 1-23, wherein R^{7b} is hydrogen.

25. The compound or salt of any one of claims 1-24, wherein R^{6b} is hydrogen or (C₁-C₆)alkyl.

26. The compound or salt of any one of claims 1-25, wherein R^{5a} is (C₁-C₁₀)alkyl, (C₂-C₁₀)alkenyl, —W—(C₃-C₇)carbocyclyl, —W-aryl, or —W-heteroaryl, wherein the C₁-C₁₀)alkyl, (C₂-C₁₀)alkenyl, —W—(C₃-C₇)carbocyclyl, —W-aryl, or —W-heteroaryl, is optionally substituted with one or more groups independently selected from Q.

27. The compound or salt of any one of claims 1-25, wherein R^{5a} is (C₂-C₁₀)alkenyl, —W—(C₃-C₇)carbocyclyl, —W-aryl, or —W-heteroaryl, wherein the (C₂-C₁₀)alkenyl, —W—(C₃-C₇)carbocyclyl, —W-aryl, or —W-heteroaryl, is optionally substituted with one or more groups independently selected from Q.

28. The compound or salt of any one of claims 1-27, wherein W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O) and wherein one more of the carbons of the (C₁-C₁₀)alkyl is optionally replaced with one or more (e.g., 1, 2, 3, 4, or 5) —O— or —NR^{w}—.

29. The compound or salt of any one of claims 1-27, wherein W is absent, (C₁-C₁₀)alkyl, or (C₂-C₆)alkynyl wherein the (C₁-C₁₀)alkyl or (C₂-C₆)alkynyl is optionally substituted with one or more (e.g., 1, 2, 3, 4, or 5) oxo (═O).

30. The compound or salt of any one of claims 1-27, wherein W is absent, —CH₂—, —C(═O)—, or propyne.

31. The compound or salt of any one of claims 1-27, wherein W is absent, —CH₂—, or —C(═O)—.

32. The compound or salt of any one of claims 1-31, wherein R^{5a} is:

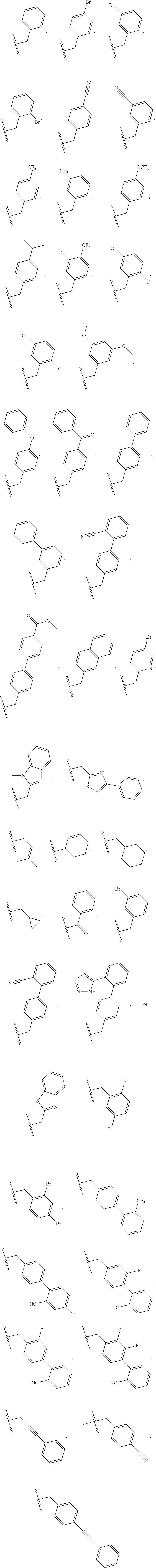

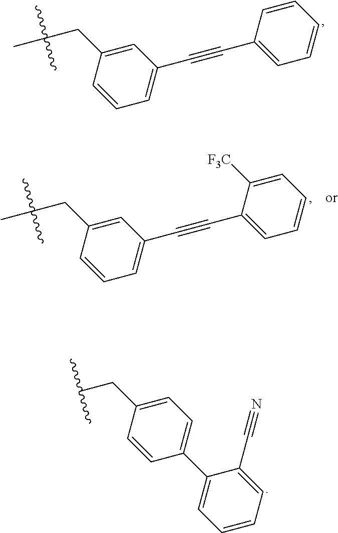 [SMILES: *Cc1cccc(C#Cc2ccccc2)c1 ; *Cc1cccc(C#Cc2ccccc2C(F)(F)F)c1 ; *Cc1ccc(-c2ccccc2C#N)cc1]

33. The compound of claim 1 that is: 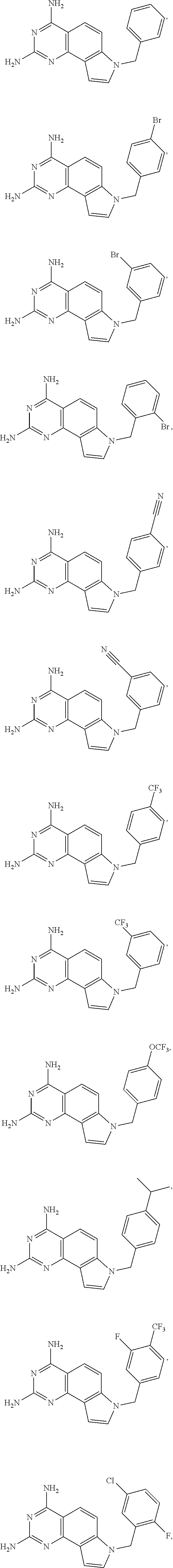 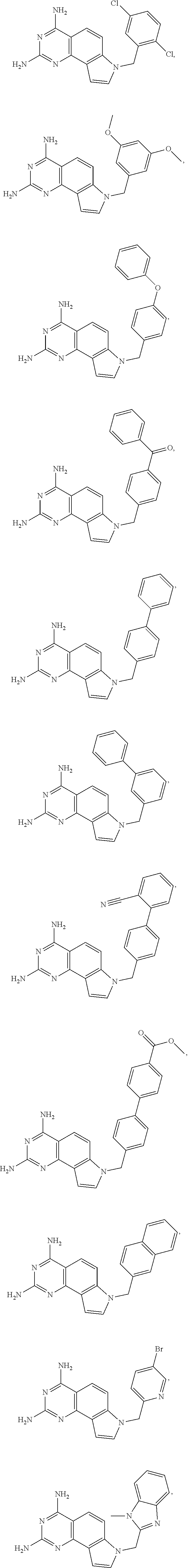 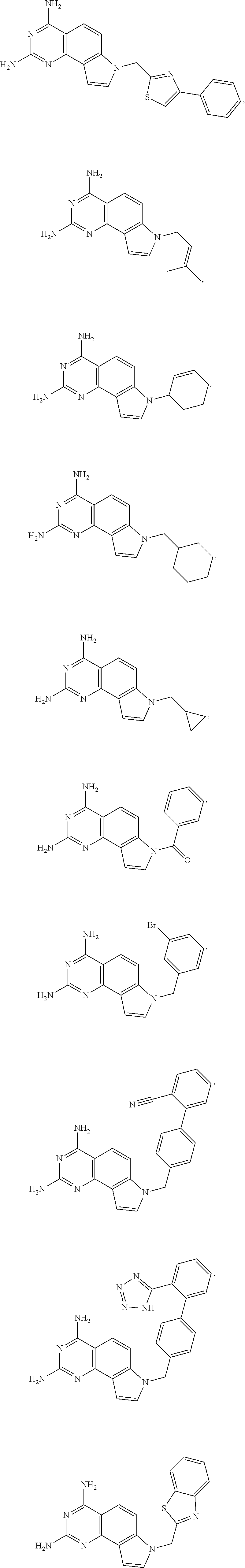 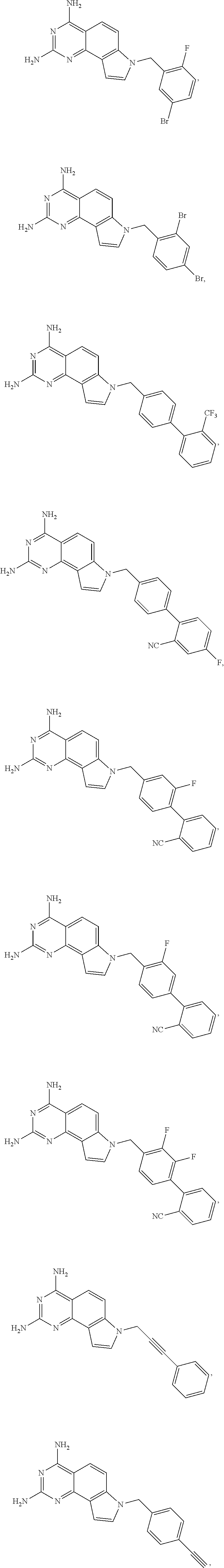 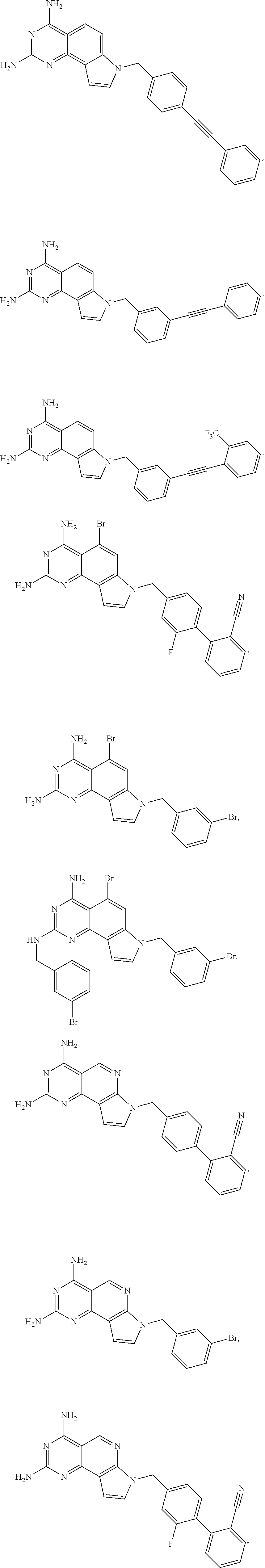 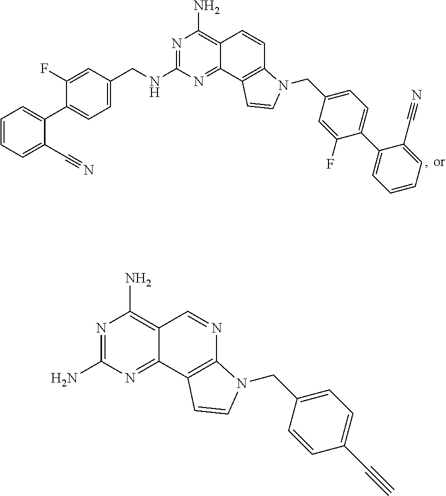 [SMILES: N#Cc1ccccc1-c1ccc(CNc2nc(N)c3ccc4c(ccn4Cc4ccc(-c5ccccc5C#N)c(F)c4)c3n2)cc1F ; C#Cc1ccc(Cn2ccc3c4nc(N)nc(N)c4cnc32)cc1]
or a salt thereof.

34. A pharmaceutical composition comprising a compound as described in any one of claims 1-33 or a pharmaceutically acceptable salt thereof, and a pharmaceutically acceptable excipient.

35. A method of treating or preventing a bacterial infection in an animal comprising administering to the animal a compound as described in any one of claims 1-33 or a pharmaceutically acceptable salt thereof.

36. The method of claim 35 wherein the animal is infected with bacteria.

37. The method of claim 35 or claim 36, wherein the bacterial infection is a N. gonorrhoeae, P. aeruginosa, A. haumannii, E. coli, K. pneumoniae, S. sonnei, P. mirabilis, MSSA or MRSA infection.

38. The method of claim 35 or claim 36, wherein the bacterial infection is a Gram-negative bacterial strain infection.

39. The method of claim 35 or claim 36, wherein the bacterial infection is a Gram-positive bacterial strain infection.

40. A compound as described in any one of claims 1-33 or a pharmaceutically acceptable salt thereof for use in medical treatment.

41. A compound as described in any one of claims 1-33 or a pharmaceutically acceptable salt thereof for the prophylactic or therapeutic treatment of a bacterial infection.

42. The use of a compound as described in any one of claims 1-33 or a pharmaceutically acceptable salt thereof for the preparation of a medicament for the prophylactic or therapeutic treatment of a bacterial infection in an animal.
